SEQUENCE LISTING (2 sequences)

SEQ ID NO: 1 - protein, 214 aa, synthetic construct
  REGION 1..214: note = Antibody light chain
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
  REGION 1..449: note = Antibody heavy chain
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0234239 A1
(43) Pub. Date: Aug. 13, 2026

(54) Anti-IL-23p19 Antibody Formulations

(71) Applicant: Boehringer Ingelheim International GmbH, Ingelheim am Rhein (DE)

(72) Inventors: Patrick Garidel, Biberach an der Riss (DE); Torsten SCHULTZ-FADEMRECHT, Aepfingen (DE)

(73) Assignee: Boehringer Ingelheim International GmbH, Ingelheim am Rhein (DE)

(21) Appl. No.: 19/411,940

(22) Filed: Dec. 8, 2025

Related U.S. Application Data
(63) Continuation of application No. 17/014,399, filed on Sep. 8, 2020, now Pat. No. 12,577,297.
(60) Provisional application No. 62/897,930, filed on Sep. 9, 2019.

(51) Int. Cl. C07K 16/24; A61K 9/00; A61K 9/08; A61K 39/00; A61K 39/395; A61K 47/12; A61K 47/26

(52) U.S. Cl. CPC C07K 16/244; A61K 9/0019; A61K 9/08; A61K 39/3955; A61K 47/12; A61K 47/26; A61K 2039/505; A61K 2039/54; C07K 2317/24; C07K 2317/51; C07K 2317/515; C07K 2317/94

(57) ABSTRACT

The present disclosure inter alia provides a liquid pharmaceutical formulation comprising: a) 150 mg/ml of an anti-IL-23p19 antibody, wherein the antibody comprises a light chain amino acid sequence according to SEQ ID NO: 1 and a heavy chain amino acid sequence according to SEQ ID NO: 2; b) a polyol; and c) a surfactant. The disclosed high concentration formulations are advantageously storage stable and suitable for subcutaneous administration.

### CROSS-REFERENCE TO RELATED APPLICATION

This application is a Continuation of U.S. application Ser. No. 17/014,399, filed Sep. 8, 2020, which claims priority from U.S. Provisional Application No. 62/897,930, filed Sep. 9, 2019, the entire contents of which are incorporated herein.

The instant application contains a Sequence Listing which has been submitted in XML format and is hereby incorporated by reference in its entirety. Said XML copy, created on Oct. 16, 2025, is named 114758-0166_SL.xml and is 3,983 bytes.

### FIELD OF THE DISCLOSURE

This invention generally relates to formulations comprising anti-IL-23p19 antibodies, such as risankizumab, which bind the p19 subunit of human IL-23. More specifically, pharmaceutical formulations comprising a high concentration of the anti-IL-23p19 antibody risankizumab, as well as related products and uses for the treatment of various diseases and disorders, are disclosed. Disclosed herein are stable liquid pharmaceutical formulations, comprising 150 mg/ml of the antibody risankizumab.

### BACKGROUND

Human IL-23 is composed of a common subunit (p40) with IL-12 and a unique p19 subunit. Despite this shared p40 subunit, the roles for IL-23 and IL-12 are quite different. IL-12 is important for Th1 responses via promotion of Th1 cell differentiation, proliferation and activation. In contrast, IL-23 supports the development and maintenance of a set of CD4⁺ T helper cells termed Th17 cells due to their ability to produce IL-17 and related cytokines. IL-23 is involved in chronic autoimmune inflammation and the modulation of IL-23 activity provides effective therapies against autoimmune diseases.

One of the autoimmune diseases where IL-23 plays a central role is psoriasis, a chronic, immune-mediated inflammatory disease characterized by the hyper proliferation of keratinocytes and skin-infiltrating T-lymphocytes that overexpress pro-inflammatory mediators. The disease is a chronic, painful immune-mediated inflammatory skin disease and has a lifelong remitting and relapsing course with varying factors that trigger exacerbations in susceptible individuals, thus making treatment challenging. The uncontrolled inflammation of psoriasis may contribute to commonly associated comorbidities, including cardiovascular (CV) disease (including hypertension and increased risk for myocardial infarction, stroke, and CV death), obesity, type 2 diabetes, arthritis, and chronic renal disease. Psoriasis is also associated with serious psychiatric comorbidities, including depression, anxiety, and suicidality, as well as substance abuse.

A highly efficient and specific inhibitor of IL-23 is the antibody risankizumab. Risankizumab is a humanized immunoglobulin G1 (IgG1) monoclonal antibody that is directed against the p19 subunit of IL-23. Binding of risankizumab to IL-23 p19 inhibits the action of IL-23 to induce and sustain T helper (Th) 17 type cells, innate lymphoid cells, 76T cells, and natural killer (NK) cells responsible for tissue inflammation, destruction and aberrant tissue repair. Risankizumab is especially effective in the treatment of autoimmune and inflammatory diseases, in particular psoriasis. Clinical studies revealed excellent safety and efficacy of risankizumab in the treatment of plaque psoriasis. The recommended dose approved for treatment of psoriasis is 150 mg which is administered subcutaneously as two 75 mg injections, on week 0, 4 and thereafter every 12 weeks.

The requirement for injection of larger medicament volumes presents a challenge, especially in patients with chronic conditions who have markedly lower rates of drug adherence and persistence than patients with acute conditions. Administration by subcutaneous route is preferred for therapeutic indications where home (self) medication is desirable, for example, for chronic diseases such as psoriasis. The subcutaneous administration route is, however, limited by the injection volume as attributable to tissue backpressure and injection pain. This also depends on the injected formulation. Most drugs that are administered by subcutaneous injection, such as risankizumab, are commonly used in unit dosages with volumes not exceeding 1 ml. Therefore, for higher volumes, such as greater than 2 ml, multiple injections are typically used, but this approach may increase the attrition rate or reduce patient adherence.

Therefore, to allow the administration of a high dose of antibody such as risankizumab with a single injection, there is a need for pharmaceutical formulations with increased antibody concentration. However, increasing the antibody concentration in antibody formulations can cause problems with stability, e.g. aggregation resulting in the formation of high molecular weight species (HMWS) and increased viscosity. Therefore, it is a great challenge to provide a stable high concentration liquid antibody formulation that is suitable for parenteral administration, such as subcutaneous injection.

### SUMMARY

The present disclosure provides a liquid antibody formulation that comprises 150 mg/ml of the antibody as defined herein. The antibody is risankizumab or an antibody comprising the same heavy and light chain sequences as risankizumab. No formulations of said antibody having such a high antibody concentration were described or available in the art and by providing such high concentration antibody formulation, the present disclosure makes an important contribution to the art. Despite the high antibody concentration, the formulations according to the present disclosure are stable and are suitable for therapeutic use. As is demonstrated in the examples, the formulations according to the present disclosure comprising 150 mg/ml of the antibody risankizumab provide advantageous stability characteristics and are well suitable for subcutaneous administration. They can provide long-term stability. Advantageously, a 150 mg dose of the antibody can be administered with a single 1 ml injection.

According to a first aspect of the present disclosure, a liquid pharmaceutical formulation is provided comprising 150 mg/ml of an anti-IL-23p19 antibody, wherein the antibody comprises a light chain amino acid sequence according to SEQ ID NO: 1 and a heavy chain amino acid sequence according to SEQ ID NO: 2.

According to a first sub-aspect of this first aspect, the liquid pharmaceutical formulation comprises
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a polyol; and
c) a surfactant.

This formulation may additionally comprise d) a buffer. Furthermore, the present disclosure provides buffer-free formulations comprising 150 mg/ml of the antibody. As is disclosed herein, the liquid pharmaceutical formulations according to the first sub-aspect are stable.

According to a second sub-aspect of this first aspect, a stable liquid pharmaceutical formulation is provided comprising
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a tonicity modifier; and
c) a surfactant, wherein the formulation has a pH of 5.5-5.9 and the formulation is isotonic.

This formulation may additionally comprise d) a buffer.

The formulations according to the first and second sub-aspect of the 150 mg/ml antibody formulation according to the first aspect may also be provided in lyophilized form.

In related aspects, a sealed container is provided which contains a formulation according to the present disclosure.

In related aspects, the present disclosure pertains to the formulations according to the present disclosure or the container containing a formulation according to the present disclosure for therapeutic treatment of a human subject. The disease to be treated may be selected from psoriasis and inflammatory bowel disease. In further embodiments, the disease to be treated may be selected from psoriatic arthritis and Crohn's disease.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1 shows the amino acid sequence of the light chain of the antibody (SEQ ID NO: 1).

FIG. 2 shows the amino acid sequence of the heavy chain of the antibody (SEQ ID NO: 2).

FIG. 3 shows an overview of the experimental schedule used to assess freeze-thaw stability of various formulations.

### DETAILED DESCRIPTION

### 150 Mg/Ml Antibody Formulation and Related Aspects

According to a first aspect, a liquid pharmaceutical formulation is provided comprising 150 mg/ml of an anti-IL-23p19 antibody, wherein the antibody comprises a light chain amino acid sequence according to SEQ ID NO: 1 and a heavy chain amino acid sequence according to SEQ ID NO: 2.

According to a first sub-aspect of this first aspect, a liquid pharmaceutical formulation is provided comprising
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a polyol; and
c) a surfactant.

The formulation according to this first sub-aspect may additionally comprise d) a buffer. Furthermore, the present disclosure provides buffer-free formulations comprising 150 mg/ml of the antibody. As is disclosed herein, the liquid pharmaceutical formulations according to the first sub-aspect are stable.

According to a second sub-aspect of this first aspect, a stable liquid pharmaceutical formulation is provided comprising
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a tonicity modifier; and
c) a surfactant,
wherein the formulation has a pH of 5.5-5.9 and the formulation is isotonic.

The stable formulation according to this second sub-aspect may additionally comprise d) a buffer.

The formulation according to the present disclosure comprises a high antibody concentration of 150 mg/ml. Despite this high antibody concentration, the liquid pharmaceutical formulations of the present disclosure are stable and advantageously can provide long-term stability. The formulations according to the present disclosure moreover address core administration challenges for a high concentration antibody formulation that is suitable for injection, by providing inter alia a suitable viscosity and good syringeability, whereby the formulation according to the present disclosure is particularly suitable for injection, such as subcutaneous injection. The advantageous properties of these formulations are demonstrated in the examples. The formulation according to the first aspect solves the challenges facing formulations for injection by providing a stable and robust formulation comprising 150 mg/ml of the antibody, thereby enabling the subcutaneous administration of a 150 mg dose of the antibody using a target volume of only 1 ml.

As disclosed herein, the formulations according to the first aspect can be provided as buffer-free or buffer-containing formulations. According to one core embodiment, the liquid pharmaceutical formulation according to the first aspect comprises d) a buffer. In another embodiment, the liquid pharmaceutical formulation does not contain a buffer as additive.

Subsequently, the components of the 150 mg/ml antibody formulation according to the first aspect are described in further detail. In particular, suitable embodiments and characteristics of components a), b), c) and optionally d) that are comprised in the formulations according to the first sub-aspect and the second sub-aspect are disclosed.

### a) The Antibody

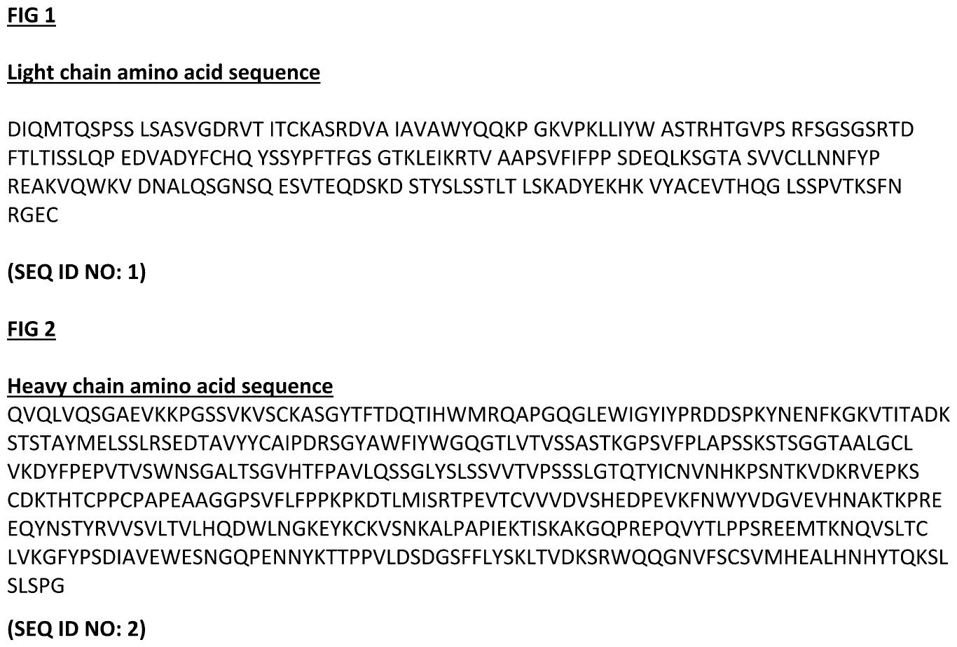

The antibody comprised in the formulation comprises a light chain amino acid sequence according to SEQ ID NO: 1 and a heavy chain amino acid sequence according to SEQ ID NO: 2. SEQ ID NO: 1 and 2 are shown in FIG. 1 and FIG. 2. The light and heavy chains of the antibody risankizumab correspond to the light and heavy chain sequences as shown in SEQ ID NO: 1 and 2. According to one embodiment, the antibody has the same light and heavy chains as the antibody risankizumab (see INN risankizumab, WHO Drug Information, Vol. 29, No. 2, 2015) and such antibody is referred to herein as risankizumab. Advantageously, the present disclosure provides stable, high concentration liquid pharmaceutical formulations for the antibody risankizumab, which is approved for the treatment of psoriasis. The whole disclosure provided herein, is specifically directed and applies to the antibody risankizumab that is comprised in the disclosed formulations. Risankizumab may be recombinantly produced in various host cells and suitable cells for recombinant antibody production are known in the art.

In one embodiment, the antibody is recombinantly produced in a mammalian cell. Suitable mammalian cells are known in the art and comprise rodent as well as human cell lines. In one embodiment, the antibody has been recombinantly produced in a hamster cell. In one embodiment, the antibody has been recombinantly produced in a CHO cell.

### Component b)

The formulation according to the first sub-aspect of the 150 mg/ml formulation according to the first aspect comprises a polyol as component b). Suitable polyols that can be used as excipient in a pharmaceutical formulation are known in the art and are described herein.

The formulation according to the second sub-aspect of the 150 mg/ml formulation according to the first aspect comprises a tonicity modifier as component b). A tonicity modifier is an agent that is suitable to adjust the tonicity of the formulation. Tonicity modifiers useful for adjusting the tonicity of a pharmaceutical formulation are known in the art, and include compounds such as salts and furthermore polyols, such as sugars and sugar alcohols. Therefore, the tonicity modifier used as component b) in the stable 150 mg/ml formulation according to the second sub-aspect may be a polyol as it is used as component b) in the 150 mg/ml formulation according to the first sub-aspect. According to one embodiment, the tonicity modifier that is comprised in the stable liquid formulation according to the second sub-aspect is thus a polyol, optionally a sugar and/or a sugar alcohol.

The term “polyol” as used herein refers to a substance with multiple hydroxyl groups, and includes sugars (reducing and nonreducing sugars) and sugar alcohols. The polyol may comprise at least three, at least four or at least five hydroxyl groups. In certain embodiments, polyols have a molecular weight that is ≤600 Da (e.g., in the range from 120 to 400 Da). A “reducing sugar” is one that contains a free aldehyde or ketone group and can reduce metal ions or react covalently with lysine and other amino groups in proteins. A “nonreducing sugar” is one that lacks a free aldehyde or ketone group and is not oxidised by mild oxidising agents such as Fehling's or Benedict's solutions. Examples for reducing and nonreducing sugars suitable for use in pharmaceutical formulations are known to the skilled person. Nonreducing sugars include e.g. sucrose and trehalose. The use of trehalose is particularly useful as is disclosed herein. Examples of sugar alcohols suitable for use in pharmaceutical formulations are known to the skilled person and include e.g. mannitol and sorbitol. The polyol may be used as tonicity agent in the formulation.

A polyol can act and may be used as tonicity modifier in order to adjust the tonicity. Certain polyols, e.g. sugars, may also act as stabilizer, thereby supporting the stability of the provided formulation.

As disclosed herein, the polyol may be selected from a sugar and a sugar alcohol. Furthermore, combinations of two or more different polyols may be used as component b), as is also demonstrated in the examples. As is shown in the examples, sugars and sugar alcohols, as well as combinations thereof, can be advantageously used in the 150 mg/ml formulation according to the present disclosure. According to one embodiment, the polyol is selected from trehalose, sucrose, sorbitol, mannitol and combinations thereof. According to one embodiment, the formulation only comprises a polyol that is selected from sugars and/or sugar alcohols as component b). According to one embodiment, the formulation only comprises a single polyol as component b).

In specific embodiments, the polyol is a sugar. The polyol may be selected from trehalose and sucrose. As is shown in the examples, the formulation may comprise trehalose as polyol and the use of trehalose is advantageous. Trehalose can be used either alone or in combination with a further polyol, e.g. a further sugar or a sugar alcohol. According to specific embodiments, the formulation only comprises a single sugar, such as trehalose, as single polyol. Using a single polyol as excipient, e.g. to adjust the tonicity, can be advantageous.

According to one embodiment, the polyol is a sugar alcohol. The sugar alcohol may be selected from sorbitol and mannitol. In embodiments, the formulation comprises mannitol as polyol. In further embodiments, the formulation comprises sorbitol. As disclosed herein, mannitol and sorbitol may either be used as single polyol, or may be used in combination either with each other or in combination with a different polyol, such as a sugar or other sugar alcohol.

Sorbitol can be used to provide a stable formulation according to the present disclosure. In certain embodiments, a sorbitol-free formulation is provided. Sorbitol-free formulations are advantageous for patients with hereditary fructose intolerance. In a specific embodiment, the liquid pharmaceutical formulation therefore does not comprise sorbitol. In certain embodiments, the formulation does not comprise a sugar alcohol.

As is demonstrated in the examples, mannitol and/or trehalose can be used as polyol in the formulation of the present disclosure, in order to adjust the desired osmolality. However, the amount of mannitol within the 150 mg/ml formulation is limited by the mannitol solubility and the amount of stock solution, which can be added during the formulation step. Therefore, in embodiments, mannitol is used in combination with a sugar, such as the highly soluble trehalose. It was found that for the antibody formulation disclosed herein, trehalose is advantageous because it is soluble enough to achieve an isotonic formulation with one excipient. Therefore, in certain embodiments, trehalose is used as polyol and trehalose may be the only polyol in the formulation that is used to adjust the isotonicity.

The polyol can be used to adjust the osmolality. In embodiments, the formulation has an osmolality in a range of 200 mOsm/kg to 400 mOsm/kg, such as in a range of 225 mOsm/kg to 375 mOsm/kg. In embodiments, the osmolality is within a range of 250 mOsm/kg to 350 mOsm/kg, such as 275 mOsm/kg to 330 mOsm/kg or 290 mOsm/kg to 320 mOsm/kg. The formulation may be isotonic, wherein “isotonic” means that the formulation of interest has essentially the same osmotic pressure as human blood. Osmolality can be measured, for example, using a vapor pressure or ice-freezing type osmometer.

The concentration of the polyol in the formulation may be at least 80 mM or at least 95 mM. In embodiments, the concentration of the polyol in the formulation is at least 115 mM, at least 125 mM, at least 135 mM, at least 140 mM, at least 150 mM or at least 160 mM. In embodiments, the concentration of the polyol in the formulation is ≤500 mM, ≤450 mM or ≤400 mM. As disclosed herein, also two or more polyols may be used as excipient b). As disclosed herein, in one core embodiment the polyol is a sugar that is used in such concentration. In one embodiment, the sugar is trehalose. The same applies with respect to the tonicity modifier that is used as component b) in the formulation according to the second sub-aspect. As disclosed herein, the tonicity modifier may be a polyol.

The concentration of the polyol in the formulation according to the first aspect, in particular the first and second sub-aspect thereof, may be in the range of 95 mM to 400 mM, such as 95 mM to 300 mM or 95 mM to 250 mM. Exemplary concentration ranges for a polyol in the formulation include, but are not limited to, 125 mM to 250 mM and 125 mM to 225 mM. The concentration of the polyol in the formulation is in one embodiment in the range of 125 mM to 225 mM. In one embodiment, the concentration of the polyol is in the range of 145 mM to 225 mM. As disclosed herein, in one core embodiment the polyol is a sugar that is used in such a concentration as described herein. In one embodiment, the sugar is trehalose.

According to one embodiment, the polyol is a sugar and wherein the concentration of the sugar is in the range of 125 mM to 250 mM, 150 mM to 250 mM, 150 mM to 200 mM or in the range of 160 mM to 200 mM. In a further embodiment, the concentration of the sugar is in the range of 170 mM to 200 mM. The concentration may be 185 mM. In one embodiment, said sugar is trehalose. Hence, also disclosed herein is a liquid pharmaceutical formulation comprising 150 mg/ml antibody and 185 mM trehalose as polyol. Trehalose may be added e.g. in the form of trehalose dihydrate.

### c) Surfactant

The liquid formulation according to the first aspect further comprises a surfactant. As is demonstrated by the examples, incorporating a surfactant in the 150 mg/ml formulation is advantageous. A surfactant is comprised as component c) in the formulation according to the first and second sub-aspect of the 150 mg/ml formulation according to the first aspect.

According to one embodiment, the surfactant is a non-ionic surfactant. Non-ionic surfactants suitable for pharmaceutical formulations are known in the art and are also described herein. The at least one surfactant may be a polysorbate (e.g. polysorbate 20) or a poloxamer (e.g. poloxamer 188). Combinations of surfactants may also be used. In one core embodiment, the surfactant is a polysorbate. The non-ionic surfactant may be selected from polysorbate 20 and/or polysorbate 80. Combinations may also be used. In one embodiment, the surfactant is polysorbate 20. In one embodiment, the formulation according to the present disclosure comprises a single surfactant, such as a single non-ionic surfactant, e.g. a single polysorbate.

In one embodiment, the concentration of the surfactant in the formulation is at least 0.05 mg/ml. The concentration may be at least 0.075 mg/ml. As is demonstrated in the examples, even low amounts of a surfactant provide a benefit. In embodiments, the surfactant concentration in the formulation is at least 0.1 mg/ml, at least 0.125 mg/ml, at least 0.15 mg/ml, at least 0.175 mg/ml or at least 0.185 mg/ml. In embodiments, the concentration of the surfactant in the formulation is ≤1 mg/ml, optionally ≤0.75 mg/ml or ≤0.5 mg/ml. In embodiments, the surfactant concentration in the formulation is ≤0.4 mg/ml, ≤0.3 mg/ml or ≤0.25 mg/ml. As disclosed herein, the surfactant may be a non-ionic surfactant. As disclosed herein, in core embodiments the surfactant is a polysorbate, optionally selected from polysorbate 20 and/or polysorbate 80. In embodiments, the surfactant is polysorbate 20. Polysorbate 20 can be advantageously used in such concentrations as disclosed herein as is demonstrated by the examples.

The concentration of the surfactant in the formulation may be in a range of 0.05 mg/ml to 0.75 mg/ml. Exemplary concentration ranges for a surfactant in the formulation include, but are not limited to, 0.05 mg/ml to 0.5 mg/ml, 0.075 mg/ml to 0.4 mg/ml or 0.075 mg/ml to 0.3 mg/ml. In embodiments, the concentration of the surfactant in the formulation is in the range of 0.05 mg/ml to 0.5 mg/ml, 0.075 mg/ml to 0.3 mg/ml or 0.1 mg/ml to 0.3 mg/ml. The concentration of the surfactant in the formulation may be 0.2 mg/ml. As is disclosed herein, the surfactant may be a non-ionic surfactant. In core embodiments the surfactant is a polysorbate, optionally selected from polysorbate 20 and/or polysorbate 80. In embodiments, the surfactant is polysorbate 20 which can be advantageously used in such concentration ranges as is demonstrated by the examples.

In specific embodiments, the formulation of the present disclosure comprises 0.2 mg/ml polysorbate 20 as surfactant. This formulation may comprise a sugar as component b), wherein the concentration of the sugar is in the range of 95 mM to 250 mM, 125 mM to 250 mM or 145 mM to 225 mM. The comprised sugar may be trehalose.

pH

The pH of the liquid pharmaceutical formulation, which in core embodiments is an aqueous formulation, may be in the range of pH 5.0 to 7.5, such as pH 5.0 to 7.0.

The pH of the liquid pharmaceutical formulation may be ≤6.8, such as ≤6.7, ≤6.6, ≤6.5, K 6.4, ≤6.3 or ≤6.2. In embodiments, the pH of the liquid pharmaceutical formulation is ≤6.1, such as ≤6.0 or ≤5.9. In embodiments, the pH of the liquid pharmaceutical formulation is ≥5.2, such as ≥5.3, ≥5.4 or ≥5.5. Exemplary ranges for the pH of the liquid pharmaceutical formulation which has a pH ≥5.2 include but are not limited to 5.2 to 6.8, such as 5.2 to 6.7, 5.2 to 6.6, 5.2 to 6.5, 5.2 to 6.4, 5.2 to 6.3 and 5.2 to 6.2. Exemplary ranges for the pH of the liquid pharmaceutical formulation which has a pH ≥5.3 include but are not limited to 5.3 to 6.8, such as 5.3 to 6.7, 5.3 to 6.6, 5.3 to 6.5, 5.3 to 6.4, 5.3 to 6.3 and 5.3 to 6.2. Exemplary ranges for the pH of a liquid pharmaceutical formulation which has a pH ≥5.4 include but are not limited to 5.4 to 6.8, such as 5.4 to 6.7, 5.4 to 6.6, 5.4 to 6.5, 5.4 to 6.4, 5.4 to 6.3 and 5.4 to 6.2. Exemplary ranges for the pH of the liquid pharmaceutical formulation which has a pH ≥5.5 include but are not limited to 5.5 to 6.8, such as 5.5 to 6.7, 5.5 to 6.6, 5.5 to 6.5, 5.5 to 6.4, 5.5 to 6.3 and 5.5 to 6.2. Exemplary ranges for the pH of the liquid pharmaceutical formulation which has a pH ≥5.6 include but are not limited to 5.6 to 6.8, such as 5.6 to 6.7, 5.6 to 6.6, 5.6 to 6.5, 5.6 to 6.4, 5.6 to 6.3 and 5.6 to 6.2. In further embodiments the pH of the formulation is in a range of 5.6 to 6.0 or 5.6 to 5.9.

According to one embodiment, the pH of the liquid pharmaceutical formulation is in the range of 5.2 to 6.5. According to one embodiment, the pH of the liquid pharmaceutical formulation is in the range of 5.2 to 6.2. Lower pH values showed less aggregation during stability and physical stress studies as can be seen from the examples.

According to one embodiment, the pH of the liquid pharmaceutical formulation is in the range of 5.5 to 6.5. In one embodiment, the pH of the liquid pharmaceutical formulation is in a range of 5.5 to 6.2.

According to one embodiment, the pH is 5.5 to 5.9. In one embodiment, the pH is 5.6 to 5.8. 150 mg/ml risankizumab formulations having such pH were tested in the examples and showed favorable characteristics.

In a further embodiment, the pH of the liquid pharmaceutical formulation is 5.7.

In a further embodiment, the pH of the liquid pharmaceutical formulation is 6.2.

As disclosed herein, the pH of the stable liquid pharmaceutical formulation according to the second sub-aspect is 5.5 to 5.9. It may be in the range of 5.5 to 5.8. In embodiments, the pH of the stable 150 mg/ml formulation according to the second sub-aspect is 5.7.

### d) Buffer

The 150 mg/ml antibody formulation according to the first aspect can be provided as buffer-free or as buffer-containing formulation. According to one core embodiment disclosed herein, the pharmaceutical formulation comprises d) a buffer. Formulations comprising a buffer showed in experiments less increase in glide forces (max and average) compared to buffer-free formulations. Accordingly, a buffer may be used as component d) in the formulation according to the first and second sub-aspect of the 150 mg/ml risankizumab formulation according to the first aspect.

A buffer can be used to maintain the solution pH of the liquid pharmaceutical formulation. Suitable buffers for pharmaceutical formulations are known in the art and are described herein. The buffer may be an organic buffer. According to one embodiment, the buffer has a pKa within 1.5 or 1 pH unit of the final pH of the liquid pharmaceutical formulation at 25° C. In certain embodiments, the buffer has a pKa in the range of pH 4.2 to 7.2 or 4.5 to 7 at 25° C. The buffer may comprise a combination of buffers. In one embodiment, a single buffer is used in the formulation as component d).

The formulation may comprise a carboxylic acid buffer as buffer d).

According to one embodiment, the buffer is selected from an acetate buffer and a succinate buffer. As is demonstrated by the examples, formulations comprising such buffers provide advantageous stability features for the high concentration formulation of the antibody that is herewith provided. In further embodiments, the buffer is a histidine buffer.

In one embodiment, the buffer is an acetate buffer. An acetate buffer may comprise sodium acetate and acetic acid. Other acetate salts may also be used in the acetate buffer.

Further buffers that may be used include but are not limited to citrate, glutamate, glycine, lactate, maleate, phosphate or tartrate buffer.

The presence of a buffer salt may support the stability of the comprised antibody which is according to the present disclosure risankizumab.

According to one embodiment, the buffer d) comprised in the formulation is not a succinate buffer. In certain embodiments, the formulation is free of a succinate buffer. In certain embodiments, a single buffer is used which is an acetate buffer, e.g. provided by an acetate salt (e.g. sodium acetate) and acetic acid.

When used, the buffer will be included in a sufficient amount to maintain the selected pH of the formulation at storage conditions for the product shelf life.

The liquid pharmaceutical formulation disclosed herein may comprise at least 1 mM, at least 2 mM buffer, at least 3 mM buffer. The buffer concentration may be at least 4 mM, at least 4.5 mM or at least 5 mM. In embodiments, the buffer concentration is 100 mM or less, such as 75 mM or less or 50 mM or less. In embodiments, the buffer concentration in the formulation is 80 mM or less, such as 75 mM or less, 70 mM or less, 60 mM or less or 50 mM or less. In further embodiments, the buffer concentration is 45 mM or less, such as 40 mM or less, 35 mM or less, 30 mM or less or 25 mM or less. In further embodiments, the buffer concentration is 20 mM or less or 15 mM or less. Exemplary concentration ranges for the comprised buffer include but are not limited to 3 mM to 100 mM, such as 4 mM to 75 mM, 4 mM to 60 mM and 4 mM to 50 mM. Further exemplary buffer concentration ranges include but are not limited to 4 mM to 45 mM, such as 5 mM to 40 mM, 5 mM to 35 mM and 5 mM to 30 mM. Still further exemplary buffer concentration ranges include but are not limited to 5 mM to 25 mM, such as 5 mM to 20 mM and 5 mM to 15 mM. In one specific embodiment, the buffer concentration is in the range of 7 mM to 12 mM. Suitable buffers are disclosed herein. In one embodiment, the formulation comprises an acetate buffer in such concentration as described.

In embodiments, the buffer concentration is 20 mM or less or 15 mM or less. In further embodiments the buffer concentration is in the range of 4 mM to 50 mM. The buffer concentration of the formulation may be in the range of 5 mM to 25 mM or 5 mM to 20 mM. The buffer concentration may also be in the range of 5 mM to 15 mM or 7 mM to 12 mM. In embodiments, the buffer concentration is 10 mM.

In certain embodiments, the formulation comprises a single buffer. In specific embodiments the single buffer is an acetate buffer.

### Specific Embodiments for Buffer-Containing Formulations Comprising 150 mg/Ml Antibody

According to one embodiment, the liquid pharmaceutical formulation comprises a) 150 mg/ml of the antibody;
b) a sugar;
c) a non-ionic surfactant; and
d) a buffer;
optionally wherein the pH of the formulation is in a range of pH 5.2 to pH 6.5, e.g. in the range of 5.2 to 6.2 or 5.5 to 6.2.

Suitable concentrations and embodiments for the excipients b) to d) were described above. In one embodiment, the concentration of the sugar is in the range of 145 mM to 225 mM and/or the concentration of the non-ionic surfactant is in the range of 0.05 mg/ml to 0.5 mg/ml or 0.075 mg/ml to 0.3 mg/ml. The sugar may be trehalose and the non-ionic surfactant may be a polysorbate, such as polysorbate 20. The pH may be 5.7. In a further embodiment the pH is 6.2.

According to one embodiment, the liquid pharmaceutical formulation comprises
a) 150 mg/ml of the antibody;
b) trehalose;
c) a polysorbate; and
d) a buffer;
optionally wherein the pH of the formulation is in a range of pH 5.2 to pH 6.5, e.g. in the range of 5.2 to 6.2 or 5.5 to 6.2.

Suitable concentrations and embodiments for the excipients b) to d) were described above. In one embodiment, the concentration of trehalose is in the range of 145 mM to 225 mM and/or the concentration of the polysorbate is in the range of 0.05 mg/ml to 0.5 mg/ml or 0.075 mg/ml to 0.3 mg/ml. The pH may be 5.7. In a further embodiment the pH is 6.2.

The buffer comprised in these liquid pharmaceutical formulations may be acetate or succinate, optionally wherein the buffer concentration is in the range of 5 mM to 25 mM. The polysorbate may be polysorbate 20.

According to one embodiment, the liquid pharmaceutical formulation comprises
a) 150 mg/ml of the antibody;
b) 170 mM to 200 mM trehalose;
c) 0.1 mg/ml to 0.3 mg/ml polysorbate, optionally polysorbate 20; and
d) a buffer, optionally wherein the buffer is an acetate buffer.

The pH of this formulation may be in a range of pH 5.2 to pH 6.5, e.g. in the range of 5.2 to 6.2 or 5.5 to 6.2.

According to one embodiment, the liquid pharmaceutical formulation comprises
a) 150 mg/ml of the antibody;
b) 185 mM trehalose;
c) 0.2 mg/ml polysorbate 20; and
d) 10 mM acetate buffer;
wherein the pH is 5.7.

This liquid formulation may be an aqueous formulation and in one embodiment, does not comprise any further additives.

### Specific Embodiments for Buffer-Free Formulations Comprising 150 mg/Ml Antibody

As disclosed herein, also buffer-free liquid pharmaceutical formulations, in particular aqueous formulations, are provided. According to one embodiment, the liquid pharmaceutical formulation comprises
a) 150 mg/ml of the antibody;
b) a polyol, optionally wherein the polyol is a sugar or sugar alcohol; and
c) a non-ionic surfactant, optionally a polysorbate;
d) no buffer.

As noted above, the present disclosure also provides buffer-free formulations and no buffer is added as excipient. At 150 mg/ml, the antibody having the light and heavy chain sequences as shown in SEQ ID NO: 1 and 2 has a high buffering capacity. Storage-stable buffer-free formulations can be provided based on the disclosure provided herein as is also shown in the examples.

In embodiments, the pH of the buffer-free formulation is in a range of pH 5.2 to pH 6.5. The pH may be in the range of 5.2 to 6.2 or 5.5 to 6.2. In one embodiment, the pH is 5.7. In a further embodiment, the pH is 6.2.

In embodiments, the buffer-free formulation comprises 80 mM to 250 mM polyol. Suitable polyols such as sugars and sugar alcohols were disclosed in detail above and it is referred to this disclosure. In one embodiment, the sugar is trehalose.

According to one embodiment, the concentration of the non-ionic surfactant in the buffer-free formulation is in the range of 0.05 mg/ml to 0.5 mg/ml, 0.075 mg/ml to 0.4 mg/ml or 0.1 mg/ml to 0.3 mg/ml. According to one embodiment, the non-ionic surfactant is a polysorbate.

It may be selected from polysorbate 20 and polysorbate 80 and is one embodiment polysorbate 20.

### Further Optional Components

In one embodiment, the liquid pharmaceutical formulation according to the present disclosure comprises an amino acid as further additive. Suitable embodiments for amino acids that can be added as excipient to a pharmaceutical formulation are known in the art and are also disclosed in the examples.

In one embodiment, the formulation comprises an amino acid which has a charged side chain, optionally a positive-charged side chain. An example of such amino acid is L-arginine.

According to one embodiment, the formulation comprises an amino acid, wherein the amino acid is present in the formulation as a salt, optionally a hydrochloride (HCl) salt.

According to one embodiment, the formulation comprises methionine. According to one embodiment, the formulation comprises amino acid L-proline.

According to one embodiment, the 150 mg/ml formulation according to the present disclosure does not comprise arginine. It was found that arginine containing formulations showed slightly elevated particle count during freeze/thaw stress studies as well as higher turbidity vales, even though there was no increase in turbidity over time. The viscosity was found to be higher. The amount of aggregates was slightly lower compared to other formulations comprising 150 mg/ml antibody but no arginine.

According to one embodiment, the formulation according to the present disclosure does not comprise an amino acid with a positive-charged side chain as excipient. According to one embodiment, the formulation according to the present disclosure does not comprise an amino acid with a charged side chain as excipient. According to one embodiment, the formulation according to the present disclosure does not comprise methionine as excipient. According to one embodiment, the formulation according to the present disclosure does not comprise an amino acid as additive.

Other excipients known in the art can be used in the formulation, as long as they do not negatively affect the stability.

However, in certain embodiments, no additional excipients are comprised in the formulation of the present disclosure. It is a particular advantage that a storage stable formulation of the antibody risankizumab can be provided with a formulation that consists essentially of or consists of a) the antibody (150 mg/ml); component b); c) a surfactant and optionally d) a buffer. As disclosed herein, it is advantageous that the formulation may comprise only a single polyol, a single surfactant and if present, a single buffer. Thereby a non-complex but nevertheless storage stable formulation is provided for the 150 mg/ml formulation of the antibody risankizumab.

### Stability Characteristics

As disclosed herein, advantageously, liquid pharmaceutical formulations comprising 150 mg/ml of the antibody are provided that are stable. Providing such stable, high-concentration formulation of the antibody risankizumab is particularly advantageous for therapeutic uses.

In the embodiments, a stable antibody formulation is a formulation wherein the antibody essentially retains its physical stability and/or biological activity upon storage. Various analytical techniques for measuring protein stability are available in the art and are disclosed herein. Stability can be measured at a selected temperature for a selected time period.

The stability characteristics of different liquid pharmaceutical formulations comprising 150 mg/ml of the antibody according to the present disclosure were tested in the examples and showed advantageous stability characteristics.

In embodiments, the stable liquid pharmaceutical formulation of the present disclosure shows no significant changes at a refrigerated temperature (2-8° C.) for at least 3 months, such as 6 months, or 1 year, or even up to 2 years or longer. A stable liquid formulation includes one that exhibits desired features at temperatures including 25° C. and 40° C. for periods including 1 month, 3 months, 6 months, 12 months, and/or 24 months.

The antibody in particular retains its physical stability in the pharmaceutical formulation, if it shows no significant increase of aggregation, precipitation and/or denaturation upon visual examination of color and/or clarity, or as measured by UV light scattering, size exclusion chromatography (SEC) and/or dynamic light scattering. The changes of protein conformation can be evaluated by fluorescence spectroscopy, which determines the protein tertiary structure, and by FTIR spectroscopy, which determines the protein secondary structure.

The antibody in particular retains its biological activity in the pharmaceutical formulation, if the biological activity of the antibody at a given time is within a predetermined range of the biological activity exhibited at the time the pharmaceutical formulation was prepared. The biological activity of the antibody can be determined, for example, by an antigen binding assay.

Aggregates can differ in origin, size, and type. Aggregates that can affect a biologic product's efficacy or safety are of particular concern, e.g. aggregates that can enhance immune responses and cause adverse clinical effects. High molecular weight aggregates, also called High Molecular Weight Species (HMWS) can be of particular concern. Aggregation can also potentially affect the subcutaneous bioavailability and pharmacokinetics of a therapeutic protein. It is advantageous that the present disclosure provides formulations, wherein the amount of high molecular weight species is low, also over extended storage times. The present disclosure in particular provides stabilized (or stable) aqueous pharmaceutical formulations as demonstrated by the reduced amounts of aggregates and/or reduced aggregate formation rates following storage. As described herein, the stability of such formulations is shown by the reduced amounts of HMWS and/or reduced HMWS formation rates following storage for varied time periods and at varied temperatures. In general, higher stability formulations are associated with lower amounts of HMWS, lower HMWS formation rates, and/or higher antibody main peaks at higher storage temperatures, relative to lower temperatures. As used herein, the term “high molecular weight species” or “HMWS” refers to higher order aggregates of the antibody of the formulations, as well as lower order aggregates of the antibody of the formulations. Lower order aggregates, include, for example, dimer species. The aggregate amounts and rates of formation may be measured or monitored by various techniques, including those disclosed in the examples.

As used herein, the term “low molecular weight species” or “LMWS” in particular refers to fragments of the antibody that are smaller than the monomer, including but not limited to free light chains, free heavy chains, molecules comprising one light chain and one heavy chain, antibody molecules missing one or both light chains, and antibody fragments obtained by cleavage of polypeptide chain(s) such as proteolytic fragments or other enzymatically or chemically degraded antibody molecules.

In certain embodiments, the antibody in the formulation disclosed herein is essentially maintained in monomeric form during storage. In particular embodiments, the formulation may fulfill one or more of the following stability characteristics:

In certain embodiments, following storage at 5° C. for 36 months, at least 94% of the antibody is present as a monomer as measured by UP-SEC, and/or the relative monomer content of the antibody does not decrease by more than 3% or more than 2.5%. In certain embodiments, following storage at 5° C. for 36 months, at least 95% or at least 96% of the antibody is present as a monomer as measured by UP-SEC, and/or the relative monomer content of the antibody does not decrease by more than 2% or more than 1.5%. In certain embodiments, following storage at 5° C. for 24 months, at least 94% of the antibody is present as a monomer as measured by UP-SEC, and/or the relative monomer content of the antibody does not decrease by more than 3% or more than 2.5%. In certain embodiments, following storage at 5° C. for 24 months, at least 95% or at least 96% of the antibody is present as a monomer as measured by UP-SEC, and/or the relative monomer content of the antibody does not decrease by more than 2% or more than 1.5% or more than 1%. In certain embodiments, following storage at 5° C. for 9 months, at least 96% or at least 96.5% of the antibody is present as a monomer as measured by UP-SEC, and/or the relative monomer content of the antibody does not decrease by more than 1.5% or more than 1%. In certain embodiments, following storage at 5° C. for 3 months, at least 96% or at least 97% of the antibody is present as a monomer as measured by UP-SEC, and/or the relative monomer content of the antibody does not decrease by more than 1% or more than 0.7% or more than 0.5%. In certain embodiments, following storage at 25° C. for 12 months, at least 90% or at least 92% of the antibody is present as a monomer as measured by UP-SEC, and/or the relative monomer content of the antibody does not decrease by more than 7% or more than 6% or more than 5%. In certain embodiments, following storage at 25° C. for 3 months, at least 95% of the antibody is present as a monomer as measured by UP-SEC, and/or the relative monomer content of the antibody does not decrease by more than 3% or more than 2%. In certain embodiments, following storage at 25° C. for 1 month, at least 96% of the antibody is present as a monomer as measured by UP-SEC, and/or the relative monomer content of the antibody does not decrease by more than 2% or more than 1%. In certain embodiments, following storage at 40° C. for 3 months, at least 87% or at least 88% of the antibody is present as a monomer as measured by UP-SEC, and/or the relative monomer content of the antibody does not decrease by more than 10% or more than 9% or more than 8%. In certain embodiments, following storage at 40° C. for 1 month, at least 93% or at least 94% of the antibody is present as a monomer as measured by UP-SEC, and/or the relative monomer content of the antibody does not decrease by more than 5% or more than 4%. In certain embodiments, following shaking at 25° C. for 21 days, at least 95% or at least 96% of the antibody is present as a monomer as measured by UP-SEC, and/or the relative monomer content of the antibody does not decrease by more than 2% or more than 1%. The decrease of the relative monomer content is calculated for the indicated storage time and temperature and in particular determined by comparing the relative monomer content at the beginning and at the end of the indicated storage. In particular embodiments, the measurements are performed as is described in the examples.

In certain embodiments, the antibody in the formulation disclosed herein does not form significant amounts of HMWS during storage. In particular, the formulation fulfills one or more of the following stability characteristics:

In certain embodiments, following storage at 5° C. for 36 months, less than 4% or less than 3% of the antibody is present as HMWS as measured by UP-SEC, and/or the relative HMWS content of the antibody does not increase by more than 2% or more than 1.5%. In certain embodiments, following storage at 5° C. for 24 months, less than 4% or less than 3% of the antibody is present as HMWS as measured by UP-SEC, and/or the relative HMWS content of the antibody does not increase by more than 2% or more than 1.5% or more than 1%. In certain embodiments, following storage at 5° C. for 9 months, less than 4% or less than 3% or less than 2.5% of the antibody is present as HMWS as measured by UP-SEC, and/or the relative HMWS content of the antibody does not increase by more than 1% or more than 0.8% or more than 0.6%. In certain embodiments, following storage at 5° C. for 3 months, less than 4% or less than 3% or less than 2.5% of the antibody is present as HMWS as measured by UP-SEC, and/or the relative HMWS content of the antibody does not increase by more than 1% or more than 0.8% or more than 0.6%. In certain embodiments, following storage at 25° C. for 12 months, less than 5% or less than 4% of the antibody is present as HMWS as measured by UP-SEC, and/or the relative HMWS content of the antibody does not increase by more than 3% or more than 2.5% or more than 2%. In certain embodiments, following storage at 25° C. for 3 months, less than 4% or less than 3.5% or less than 3.2% of the antibody is present as HMWS as measured by UP-SEC, and/or the relative HMWS content of the antibody does not increase by more than 2% or more than 1.5%. In certain embodiments, following storage at 25° C. for 1 month, less than 4% or less than 3.5% or less than 3% of the antibody is present as HMWS as measured by UP-SEC, and/or the relative HMWS content of the antibody does not increase by more than 1.5% or more than 1%. In certain embodiments, following storage at 40° C. for 3 months, less than 6.5% or less than 6% or less than 5.5% of the antibody is present as HMWS as measured by UP-SEC, and/or the relative HMWS content of the antibody does not increase by more than 5% or more than 4%. In certain embodiments, following storage at 40° C. for 1 month, less than 5% or less than 4.5% or less than 4% of the antibody is present as HMWS as measured by UP-SEC, and/or the relative HMWS content of the antibody does not increase by more than 2.5% or more than 2%. In certain embodiments, following shaking at 25° C. for 21 days, less than 3% or less than 2% of the antibody is present as HMWS as measured by UP-SEC, and/or the relative HMWS content of the antibody does not increase by more than 2% or more than 1.5% or more than 1%. The increase of the relative HMWS content is calculated for the indicated storage time and temperature and in particular determined by comparing the relative HMWS content at the beginning and at the end of the indicated storage. In particular embodiments, the measurements are performed as is described in the examples.

In further embodiments, the antibody in the formulation disclosed herein does not form significant amounts of LMWS during storage. In particular embodiments, the formulation may fulfill one or more of the following stability characteristics:

In certain embodiments, following storage at 5° C. for 36 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 24 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 9 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 3 months, less than 2% or less than 1.5% or less than 1% of the antibody is present as LMWS as measured by UP-SEC, and/or the relative LMWS content of the antibody does not increase by more than 1% or more than 0.5% or more than 0.25%. In certain embodiments, following storage at 25° C. for 12 months, less than 6% or less than 5% or less than 4.5% of the antibody is present as LMWS as measured by UP-SEC, and/or the relative LMWS content of the antibody does not increase by more than 5% or more than 4% or more than 3%. In certain embodiments, following storage at 25° C. for 3 months, less than 3% or less than 2% or less than 1.8% of the antibody is present as LMWS as measured by UP-SEC, and/or the relative LMWS content of the antibody does not increase by more than 2% or more than 1.5% or more than 1%. In certain embodiments, following storage at 25° C. for 1 month, less than 2% or less than 1.5% or less than 1.2% of the antibody is present as LMWS as measured by UP-SEC, and/or the relative LMWS content of the antibody does not increase by more than 1% or more than 0.6% or more than 0.4%. In certain embodiments, following storage at 40° C. for 3 months, less than 8% or less than 7% or less than 6% of the antibody is present as LMWS as measured by UP-SEC, and/or the relative LMWS content of the antibody does not increase by more than 8% or more than 7% or more than 6%. In certain embodiments, following storage at 40° C. for 1 month, less than 4% or less than 3.5% or less than 3% of the antibody is present as LMWS as measured by UP-SEC, and/or the relative LMWS content of the antibody does not increase by more than 3% or more than 2.5% or more than 2.2%. The increase of the relative LMWS content is calculated for the indicated storage time and temperature and in particular determined by comparing the relative LMWS content at the beginning and at the end of the indicated storage. In particular embodiments, the measurements are performed as is described in the examples.

In certain embodiments, the relative amount of the antibody being in monomeric form, HMWS and/or LMWS is determined using UP-SEC, in particular as described in the examples. For example, an ultra-performance liquid chromatography (UPLC) system such as an Acquity UPLC system of Waters (Milford, MA, USA) comprising a size exclusion chromatography (SEC) column is used. Proteins eluting from the SEC column may be detected by UV absorption at 280 nm and determining of the relative amounts may be done by calculating the area under the curve (AUC) for each elution peak. Peaks may be assigned to the different species by their elution time corresponding to the molecular size of the species. For measuring the relative monomer content, relative HMWS content and/or relative LMWS content of the antibody in the formulation, in particular the monomeric antibody, HMWS and LMWS are separated from each other, if present in the formulation. In particular, the relative content or amount is indicated as a percentage value and the sum of monomeric antibody, HMWS and LMWS is 100%.

In certain embodiments, the turbidity or opalescence of the formulation disclosed herein does not significantly increase during storage. In particular embodiments, the formulation may fulfill one or more of the following stability characteristics:

In certain embodiments, following storage at 5° C. for at least 36 months, the formulation has an opalescence of 12 FNU (Formazin Nephelometry Units) or less or 10 FNU or less, and/or the opalescence does not increase by more than 5 FNU or more than 3 FNU. In certain embodiments, following storage at 5° C. for at least 3, 6, 9, 12, 18 or 24 months, the formulation has an opalescence of 12 FNU (Formazin Nephelometry Units) or less or 10 FNU or less, and/or the opalescence does not increase by more than 5 FNU or more than 3 FNU. In certain embodiments, following storage at 25° C. for at least 1, 3, 6, 9 or 12 months, the formulation has an opalescence of 12 FNU or less or 10 FNU or less, and/or the opalescence does not increase by more than 7 FNU or more than 5 FNU. In certain embodiments, following storage at 40° C. for at least 1 or 3 months, the formulation has an opalescence of 12 FNU or less or 10 FNU or less, and/or the opalescence does not increase by more than 5 FNU or more than 3 FNU. In certain embodiments, following shaking at 25° C. for 21 days, the formulation has an opalescence of 12 FNU or less or 10 FNU or less, and/or the opalescence of the formulation does not increase by more than 3 FNU or more than 2 FNU. The increase of the opalescence is calculated for the indicated storage time and temperature and in particular determined by comparing the opalescence at the beginning and at the end of the indicated storage. In particular embodiments, the measurements are performed as is described in the examples.

In certain embodiments, the opalescence or turbidity is measured according to pharmacopeia or according to industrial standard ISO 7027. In certain embodiments, the opalescence or turbidity of the formulation is determined using a nephelometer such as a HACH Lange opalescence meter of Hach-Lange GmbH (Germany), in particular as described in the examples. Opalescence may be measured at different wavelengths, including at 400-600 nm. In embodiments, the FNA values indicated above are measured at 400-600 nm. Higher FNU values indicate a higher opalescence and turbidity.

In certain embodiments, the antibody in the formulation disclosed herein does not form significant additional amounts of acidic or basic variants during storage. In particular embodiments, the formulation may fulfill one or more of the following stability characteristics:

In certain embodiments, following storage at 5° C. for 36 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC). In certain embodiments, following storage at 5° C. for 24 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC). In certain embodiments, following storage at 5° C. for 6 months, at least 60% or at least 65% of the antibody is present as main peak variants, and/or the relative content of main peak variants of the antibody does not decrease by more than 5% or more than 4%, as determined by ion exchange chromatography (IEC). In certain embodiments, following storage at 5° C. for 3 months, at least 60% or at least 65% of the antibody is present as main peak variants, and/or the relative content of main peak variants of the antibody does not decrease by more than 4% or more than 3% or more than 2%, as determined by ion exchange chromatography (IEC). In certain embodiments, following storage at 25° C. for 12 months, at least 35% or at least 40% or at least 45% of the antibody is present as main peak variants, and/or the relative content of main peak variants of the antibody does not decrease by more than 35% or more than 30% or more than 25%, as determined by ion exchange chromatography (IEC). In certain embodiments, following storage at 25° C. for 3 months, at least 55% or at least 60% of the antibody is present as main peak variants, and/or the relative content of main peak variants of the antibody does not decrease by more than 15% or more than 10%, as determined by ion exchange chromatography (IEC). In certain embodiments, following storage at 25° C. for 1 month, at least 60% or at least 65% of the is present as main peak variants, and/or the relative content of main peak variants of the antibody does not decrease by more than 10% or more than 5%, as determined by ion exchange chromatography (IEC). In certain embodiments, following storage at 5° C. for 36 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 24 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 6 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 3 months, less than 30% or less than 28% of the antibody is present as acidic peak group variants, and/or the relative content of acidic peak group variants of the antibody does not increase by more than 3% or more than 2% or more than 1%, as determined by ion exchange chromatography (IEC). In certain embodiments, following storage at 25° C. for 12 months, less than 50%, less than 45% or less than 40% of the antibody is present as acidic peak group variants, and/or the relative content of acidic peak group variants of the antibody does not increase by more than 30% or more than 25% or more than 20%, as determined by ion exchange chromatography (IEC). In certain embodiments, following storage at 25° C. for 3 months, less than 40% or less than 35% or less than 30% of the antibody is present as acidic peak group variants, and/or the relative content of acidic peak group variants of the antibody does not increase by more than 10% or more than 8% or more than 6%, as determined by ion exchange chromatography (IEC). In certain embodiments, following storage at 25° C. for 1 month, less than 35% or less than 30% or less than 28% of the antibody is present as acidic peak group variants, and/or the relative content of acidic peak group variants of the antibody does not increase by more than 4% or more than 3%, as determined by ion exchange chromatography (IEC). In certain embodiments, following storage at 5° C. for 36 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC). In certain embodiments, following storage at 5° C. for 24 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC). In certain embodiments, following storage at 5° C. for 6 months, less than 15% or less than 10% of the antibody is present as basic peak group variants, and/or the relative content of basic peak group variants of the antibody does not increase by more than 4% or more than 3% or more than 2%, as determined by ion exchange chromatography (IEC). In certain embodiments, following storage at 5° C. for 3 months, less than 15% or less than 10% of the antibody is present as basic peak group variants, and/or the relative content of basic peak group variants of the antibody does not increase by more than 3% or more than 2%, as determined by ion exchange chromatography (IEC). In certain embodiments, following storage at 25° C. for 12 months, less than 30% or less than 25% or less than 22% of the antibody is present as basic peak group variants, and/or the relative content of basic peak group variants of the antibody does not increase by more than 25% or more than 20% or more than 15%, as determined by ion exchange chromatography (IEC). In certain embodiments, following storage at 25° C. for 3 months, less than 20% or less than 15% or less than 12% of the antibody is present as basic peak group variants, and/or the relative content of basic peak group variants of the antibody does not increase by more than 9% or more than 7% or more than 5%, as determined by ion exchange chromatography (IEC). In certain embodiments, following storage at 25° C. for 1 month, less than 15% or less than 10% or less than 9% of the antibody is present as basic peak group variants, and/or the relative content of basic peak group variants of the antibody does not increase by more than 3% or more than 2%, as determined by ion exchange chromatography (IEC). The decrease of the relative content of the main peak variants and the increase of the relative content of the acidic and basic peak group variants are calculated for the indicated storage time and temperature and in particular determined by comparing the relative content of the respective peak variants at the beginning and at the end of the indicated storage. In particular embodiments, the measurements are performed as is described in the examples.

In certain embodiments, the relative amount of the antibody being main peak variants, acidic peak variants and/or basic peak variants is determined using ion exchange chromatography (IEC), in particular as described in the examples. In particular, weak cation exchange chromatography (WCX) is used. For example, a high-performance liquid chromatography (HPLC) system such as an Alliance HPLC system of Waters (Milford, MA, USA) comprising a WCX column is used. Proteins eluting from the WCX column may be detected by UV absorption at 280 nm and determining of the relative amounts may be done by calculating the area under the curve (AUC) for each elution peak or each group of elution peaks. Peaks may be assigned to the different species by their elution conditions corresponding to the surface charge of the antibody species. The main peak is the largest peak in an IEC chromatogram of the non-degraded antibody. For the stability analysis, the measurement can be performed after preparation of the formulation (TO) and then after the indicated storage time under the indicated storage condition. The acidic peak group (APG) includes all peaks prior to the main peak. These peaks include antibody variants which are more acidic than the native antibody variants of the main peak, and/or which have more negative charges on their surface under the chromatography conditions. The basic peak group includes all peaks after the main peak. These peaks include antibody variants which are more acidic than the native antibody variants of the main peak, and/or which have more positive charges on their surface under the chromatography conditions. For measuring the relative amount of main peak variants, acidic peak group variants and/or basic peak group variants of the antibody in the formulation, in particular the main peak is separated from the acidic peak group and the basic peak group, if present in the formulation. In particular, the relative content or amount is indicated as a percentage value and the sum of main peak variants, acidic peak group variants and basic peak group variants is 100%.

In certain embodiments, the antibody in the formulation disclosed herein essentially maintains its specific binding activity to human IL-23 during storage. In particular embodiments, the formulation fulfills one or more of the following stability characteristics:

In certain embodiments, following storage at 5° C. for 36 months, at least 95% or at least 97% of the specific binding activity to IL-23 is measured compared to a reference antibody, wherein the reference antibody has not been stored. In certain embodiments, following storage at 5° C. for 4, 6, 9, 12, 18 or 24 months, at least 95% or at least 97% of the specific binding activity to IL-23 is measured compared to a reference antibody, wherein the reference antibody has not been stored. In certain embodiments, following storage at 25° C. for 2, 3, 4, 6, 9, 12, or 18 months, at least 93% or at least 96% of the specific binding activity to IL-23 is measured compared to a reference antibody, wherein the reference antibody has not been stored. In certain embodiments, following storage at 40° C. for 3, 4 or 6 months, at least 90% or at least 95% of the specific binding activity to IL-23 is measured compared to a reference antibody, wherein the reference antibody has not been stored. In particular embodiments, the measurements are performed as is described in the examples.

In certain embodiments, the specific binding activity to human IL-23 of the antibody in the formulation is determined using surface plasmon resonance measurements, for example using a Biacore instrument such as Biacore T200 of GE Healthcare Life Science (United Kingdom), in particular as described in the examples.

### Further Characteristics of the Liquid Pharmaceutical Formulation According to the First Aspect

In advantageous embodiments, the liquid pharmaceutical formulation of the present disclosure is an aqueous formulation. All liquid formulations disclosed herein are in one embodiment an aqueous formulation. The following description applies to the 150 mg/ml formulation according to the first aspect and therefore, also applies to the formulations according to the first and second sub-aspect as disclosed herein, unless the specific context indicates otherwise.

According to one embodiment the dynamic viscosity of the liquid pharmaceutical formulation according to the first aspect measured at 20° C. is ≤30 mPas (mPa s), such as ≤25 mPas or ≤20 mPas. In embodiments, the dynamic viscosity of the formulation measured at 20° C. is ≤18 mPas, such as ≤16 mPas, ≤15 mPas, ≤14 mPas, ≤13 mPas or ≤12 mPas. In specific embodiments, the dynamic viscosity is such that the formulation is suitable for subcutaneous administration, as is also shown in the examples. The dynamic viscosity may be determined as described in the examples.

According to one embodiment, the formulation of the present disclosure has a conductivity in a range of 0.8 to 5 mS/cm. In embodiments, the conductivity range is 1 to 2 mS/cm or 1.2 to 1.8 mS/cm. In embodiments, the formulation is characterized in that the change of conductivity over a storage time of at least 12 months at 25° C. is ≤1 mS/cm, such as ≤0.75 mS/cm, ≤0.5 mS/cm or ≤0.3 mS/cm.

The liquid formulation according to the present disclosure is a pharmaceutical formulation. A pharmaceutical formulation in particular refers to compositions which are in such form as to permit the active ingredient (here the antibody comprising a light chain as shown in SEQ ID NO: 1 and a heavy chain as shown in SEQ ID NO: 2) to be effective, and which contains no additional components which are toxic to the subjects to which the formulation would be administered.

The formulations according to the first aspect disclosed herein are advantageously suitable for parenteral delivery. Parenteral administration includes e.g. subcutaneous, intramuscular, intradermal, intramedullary injections, as well as intrathecal, direct intraventricular, intravenous, intraperitoneal and intravitreal. Drugs can be administered in a variety of conventional ways, such as intraperitoneal, parenteral, intraarterial or intravenous injection. In one embodiment, the disclosed formulation is an injectable formulation. The formulation disclosed herein is in embodiments suitable for subcutaneous, intravenous, or intramuscular administration. Advantageously, the disclosed formulation is suitable for subcutaneous injection. The 150 mg/ml formulation disclosed herein is particularly advantageous, because overall characteristics are achieved which makes the formulation particularly suitable for subcutaneous administration. The high concentration allows to administer a small volume of the formulation while still achieving a high antibody dose (here e.g. 1 ml for 150 mg dose). Furthermore, the formulations according to the present disclosure show a good syringeability. They moreover have advantageous viscosity and osmolality characteristics and achieve good glide forces (max and average), also upon storage as is disclosed in the examples. In embodiments, the liquid pharmaceutical formulation according to the present disclosure is isotonic with the intended site of administration. For example, if the formulation is intended for administration parenterally, it can be isotonic with blood (which is about 300 mOsm/kg osmolality). Suitable osmolality ranges are described elsewhere.

The liquid antibody formulation can be made by taking the drug substance which is in liquid form (e.g., in an aqueous pharmaceutical formulation) and buffer exchanging and preparing it into the desired buffer as the last step of the purification process. The drug substance in the final buffer may be concentrated to a desired concentration or a more concentrated form of the antibody is diluted to achieve the 150 mg/ml concentration. Concentration of the formulation can be carried out by any suitable method. In one aspect, the concentration process can include ultrafiltration.

The liquid pharmaceutical formulation according to the first aspect is in one core embodiment not a formulation that has been prepared by reconstituting a lyophilized formulation. In this core embodiment, there is no lyophilization step during the preparation of the liquid pharmaceutical formulation. Excipients such as component b) and surfactant c) may be added to the drug substance which may be diluted using the appropriate buffer to final protein concentration of 150 mg/ml. A pharmaceutical formulation to be used for in vivo administration typically is sterile. In certain embodiments, this may be accomplished by filtration through sterile filtration membranes. The final formulated drug substance may thus be filtered (e.g. using 0.22 pm filters) and may be filled into a final container (e.g. glass vials or syringe). The prepared liquid formulation is in this embodiment for direct administration to the patient so that there is no lyophilization or reconstitution step. Such liquid pharmaceutical formulations are disclosed herein and were also made and analysed in the examples.

### Lyophilized and Reconstituted Pharmaceutical Formulations

According to one embodiment, the liquid pharmaceutical formulation according to the first aspect is prepared from a lyophilized pharmaceutical formulation by reconstitution. In embodiments, the liquid pharmaceutical composition described herein is thus a reconstituted formulation. This applies to the liquid formulations according to the first and second sub-aspect of the 150 mg/ml antibody formulation according to the first aspect.

The terms “lyophilization,” or “lyophilized” in particular refer to a process by which the material to be dried is first frozen and then the ice or frozen solvent is removed by sublimation in a vacuum environment. Such technologies are well-known in the art and therefore, are not described in detail herein. An excipient may be included in pre-lyophilized formulations to enhance stability of the lyophilized product upon storage. The lyophilized formulation may comprise a cryoprotectant, which generally includes agents which provide stability to the protein against freezing-induced stresses. They may also offer protection during primary and secondary drying, and long-term product storage. Examples include sugars such as sucrose and trehalose and surfactants such as polysorbates. The lyophilized formulation may also include a lyoprotectant, which includes agents that provide stability to the protein during the drying or dehydration process (primary and secondary drying cycles). This helps to maintain the protein conformation, minimize protein degradation during the lyophilization cycle and improve the long-term product stability. Examples include polyols, such as sugars, e.g. sucrose and trehalose. The liquid pharmaceutical formulations disclosed according to the first aspect comprise excipients that qualify as cryo- and/or lyoprotectant. accordingly, lyophilized formulations may be prepared from such formulations. In an embodiment, the antibody risankizumab is formulated as a lyophilized powder for reconstituting and utilizing for intravenous administration.

A “reconstituted” formulation is one that has been prepared by dissolving a lyophilized pharmaceutical antibody formulation in a diluent such that the antibody is dispersed in the reconstituted formulation. The reconstituted formulation is suitable for administration, and may optionally be suitable for subcutaneous administration.

The lyophilized pharmaceutical formulation is prepared in anticipation of reconstitution at the desired concentration, here 150 mg/ml of the antibody.

According to one embodiment, a lyophilized formulation of an anti-IL-23p19 antibody is provided, wherein the antibody comprises a light chain amino acid sequence according to SEQ ID NO: 1 and a heavy chain amino acid sequence according to SEQ ID NO: 2. According to one embodiment, the lyophilized formulation of the antibody risankizumab is defined in terms of the solution used to make the lyophilized formulation, e.g. the pre-lyophilization solution. This lyophilized formulation is made by lyophilizing the liquid 150 mg/ml antibody formulation according to the first aspect, such as the liquid pharmaceutical formulation according to the first aspect as defined in any one of the below embodiments 1 to 86. As disclosed herein, the liquid formulation is in one embodiment an aqueous formulation. Such aqueous formulation may be used to prepare the lyophilized pharmaceutical formulation.

In yet other embodiments, the lyophilized formulation of the antibody risankizumab is defined in terms of the reconstituted solution generated from the lyophilized formulation. According to one embodiment, a lyophilized formulation of an anti-IL-23p19 antibody is thus provided, wherein the antibody comprises a light chain amino acid sequence according to SEQ ID NO: 1 and a heavy chain amino acid sequence according to SEQ ID NO: 2, said lyophilized formulation providing upon reconstitution the liquid 150 mg/ml antibody formulation according to the first aspect, in particular the first and second sub-aspect thereof. According to embodiments, the lyophilized formulation provides upon reconstitution the liquid pharmaceutical formulation as defined in any one of the below embodiments 1 to 86 or 104 to 119. This risankizumab formulation may be an aqueous formulation.

Also provided is a lyophilized formulation comprising
a) an anti-IL-23p19 antibody, wherein the antibody comprises a light chain amino acid sequence according to SEQ ID NO: 1 and a heavy chain amino acid sequence according to SEQ ID NO: 2, in an amount that upon reconstitution provides an antibody concentration of 150 mg/ml;
b) a polyol;
c) a surfactant; and
d) optionally a buffer.

In one embodiment, the lyophilized pharmaceutical formulation comprises 150 mg of the antibody. The antibody is risankizumab.

Also provided is a lyophilized formulation comprising
a) an anti-IL-23p19 antibody, wherein the antibody comprises a light chain amino acid sequence according to SEQ ID NO: 1 and a heavy chain amino acid sequence according to SEQ ID NO: 2, in an amount that upon reconstitution provides an antibody concentration of 150 mg/ml;
b) a tonicity modifier;
c) a surfactant; and
d) optionally a buffer.

In one embodiment, the lyophilized pharmaceutical formulation comprises 150 mg of the antibody. The antibody is risankizumab.

The comprised components such as suitable polyols for pharmaceutical formulations were already disclosed above in conjunction with the liquid pharmaceutical formulation and it is referred to the above disclosure which also applies here. Suitable polyols include sugars and sugar alcohols, which may also be used in combination. The polyol may have one or more of the characteristics as defined in any one of the below embodiments 6 to 13 of the liquid pharmaceutical formulation according to the first aspect. In one embodiment, the polyol is a sugar, optionally selected from trehalose and sucrose. In one embodiment, the sugar is trehalose.

Suitable surfactants were already disclosed above in conjunction with the liquid pharmaceutical formulation and it is referred to the above disclosure which also applies here. The surfactant may have one or more of the characteristics as defined in any one of the below embodiments 22 to 25 of the liquid pharmaceutical formulation according to the first aspect. In one embodiment, the surfactant is a polysorbate, optionally selected from polysorbate 20 and 80. In one embodiment, the polysorbate is polysorbate 20.

The lyophilized formulation comprises in one embodiment a buffer. Buffers suitable to prepare lyophilized formulations are known in the art and suitable buffers were also disclosed above in conjunction with the liquid pharmaceutical formulation according to the first aspect and it is referred to the above disclosure.

According to one embodiment, the lyophilized formulation is characterized in that the formulation has upon reconstitution a pH as disclosed herein for the liquid pharmaceutical formulation according to the first aspect. Suitable pH values were disclosed above and it is referred to the respective disclosure which also applies here. Upon reconstitution, the pH may be as defined in any one of the below embodiments 31 to 36 of the liquid 150 mg/ml pharmaceutical formulation. Furthermore, upon reconstitution, the pH may be as defined for the formulation according to second sub-aspect. The pH upon reconstitution may be 5.5 to 5.9, e.g. 5.6 to 5.8.

The lyophilized risankizumab formulations of the present disclosure are reconstituted prior to administration. In some instances, it may be desirable to lyophilize the risankizumab formulation in the container in which reconstitution of the antibody is to be carried out in order to avoid a transfer step.

### Containers and Uses

According to a further aspect of the present disclosure, a sealed container is provided which contains the liquid pharmaceutical formulation or the lyophilized pharmaceutical formulation according to the first aspect of the present disclosure. The container may be a vial or pre-filled syringe. In embodiments, the container contains 2 ml or less of the liquid pharmaceutical formulation, optionally 1.5 ml or less or 1 ml or less. Such container may comprise the advantageous stable liquid pharmaceutical formulation according to the first or second sub-aspect of the 150 mg/ml antibody formulation according to the first aspect.

In core embodiments, the container such as the syringe comprises a single dose of 150 mg antibody. As disclosed herein, the antibody is risankizumab.

In one embodiment, the liquid pharmaceutical formulation according to the first aspect of the present disclosure is comprised in a syringe which is equipped with a needle. In particular embodiments, the needle is suitable for subcutaneous administration. The needle may be a 27 Gauge spinal thin-wall needle or other needle suitable for subcutaneous use.

According to one embodiment the pre-filled syringe equipped with a needle has an average gliding force that is 20 N or less. In embodiments, the average gliding force is in the range of 5 to 20 N or 5 to 15 N. In embodiments, the pre-filled syringe has a break loose force of 3 to 12 N, preferably 3 to 9 N.

In certain embodiments, the syringe equipped with a needle and comprising the liquid pharmaceutical formulation according to the first aspect essentially maintains the maximum and/or average gliding force needed to eject it from a syringe during storage. In certain embodiments, following storage at 5° C. for 36 months, the maximum gliding force of the syringe pre-filled with the liquid formulation is 14 N or lower, 12 N or lower or 10 N or lower, and/or the maximum gliding force does not increase by more than 5 N or more than 4 N or more than 3 N. In certain embodiments, following storage at 5° C. for 24 months, the maximum gliding force of the syringe pre-filled with the liquid formulation is 14 N or lower, 12 N or lower, 10 N or lower or 8 N or lower, and/or the maximum gliding force does not increase by more than 3 N or more than 2 N or more than 1.5 N. In certain embodiments, following storage at 5° C. for 9 months, the maximum gliding force is 9 N or lower or 8 N or lower, and/or the maximum gliding force does not increase by more than 2 N or more than 1.5 N or more than 1 N. In certain embodiments, following storage at 5° C. for 3 months, the maximum gliding force is 8 N or lower or 7.5 N or lower, and/or the maximum gliding force does not increase by more than 1.5 N or more than 1 N. In certain embodiments, following storage at 25° C. for 3 months, the maximum gliding force is 10 N or lower or 8 N or lower, and/or the maximum gliding force does not increase by more than 3 N or more than 2 N or more than 1.5 N. In certain embodiments, following storage at 25° C. for 1 month, the maximum gliding force of the pre-filled syringe equipped with a needle is 8 N or lower or 7.5 N or lower, and/or the maximum gliding force does not increase by more than 1.5 N or more than 1 N. In certain embodiments, following storage at 40° C. for 1 month, the maximum gliding force is 16 N or lower or 13 N or lower, and/or the maximum gliding force does not increase by more than 10 N or more than 8 N or more than 6 N.

In certain embodiments, following storage at 5° C. for 36 months, the average gliding force of the syringe prefilled with the liquid formulation according to the first aspect and equipped with a needle is 14 N or lower, 12 N or lower, 10 N or lower or 9 N or lower, and/or the average gliding force does not increase by more than 5 N or more than 4 N or more than 3 N. In certain embodiments, following storage at 5° C. for 24 months, the average gliding force of the syringe prefilled with the liquid formulation according to the first aspect and equipped with a needle is 14 N or lower, 12 N or lower, 10 N or lower or 8 N or lower, and/or the average gliding force does not increase by more than 3 N or more than 2 N or more than 1.5 N. In certain embodiments, following storage at 5° C. for 9 months, the average gliding force of the pre-filled syringe equipped with the needle is 9 N or lower or 7.5 N or lower, and/or the average gliding force does not increase by more than 2 N or more than 1.5 N or more than 1 N. In certain embodiments, following storage at 5° C. for 3 months, the average gliding force is 8 N or lower or 7 N or lower, and/or the average gliding force does not increase by more than 1.5 N or more than 1 N or no more than 0.5 N. In certain embodiments, following storage at 25° C. for 12 months, the average gliding force is 15 N or lower or 13 N or lower, and/or the average gliding force does not increase by more than 9 N or more than 8 N or more than 7 N. In certain embodiments, following storage at 25° C. for 3 months, the average gliding force is 9 N or lower or 8 N or lower, and/or the average gliding force does not increase by more than 3 N or more than 2 N or more than 1.5 N. In certain embodiments, following storage at 25° C. for 1 month, the average gliding force is 8 N or lower or 7 N or lower, and/or the average gliding force does not increase by more than 1.5 N or more than 1 N or no more than 0.5 N. In certain embodiments, following storage at 40° C. for 3 month, the average gliding force is 18 N or lower or 15 N or lower, and/or the average gliding force does not increase by more than 12 N or more than 10 N or more than 9 N. In certain embodiments, following storage at 40° C. for 1 month, the average gliding force is 13 N or lower or 10 N or lower, and/or the average gliding force does not increase by more than 7 N or more than 5 N or more than 3 N.

The increase of the maximum or relative gliding force is calculated for the indicated storage time and temperature and in particular determined by comparing the maximum or relative gliding force at the beginning and at the end of the indicated storage. In particular embodiments, the measurements are performed as is described in the examples.

The maximum gliding force of the formulation refers to the maximum mechanical force needed to eject the formulation from a syringe. The average gliding force of the formulation refers to the average mechanical force needed to eject the formulation from a syringe. In some embodiments, the gliding force is determined according to industrial norms such as ISO 7886, ISO 11040 and ISO 11499. In certain embodiments, the maximum and average gliding force of the formulation is determined using a tensile and compression testing machine such as a Zwick 2.5 TS/N of Zwick (Germany), in particular as described in the examples. The measurement may be performed using a 1 ml syringe with a 27 gauge×½ inch needle, such as a Neopak 1 ml syringe of Becton Dickinson (USA), in particular a syringe equipped with a needle as used in the examples. The measurement may be performed using a speed of about 300 to 500 mm/min such as about 380 mm/min, in particular 379.2 mm/min, for example for 5 seconds.

A further aspect according to the present disclosure pertains to the liquid pharmaceutical formulations or the lyophilized pharmaceutical formulations according to the first aspect or the container according to the further aspect disclosed herein for therapeutic treatment of a human subject. The disease to be treated is a disease that can be treated with an anti-IL-23p19 antibody and such diseases are known in the art. The disease may be selected from the group consisting of inflammatory diseases, autoimmune diseases, respiratory diseases, metabolic disorders and cancer. In embodiments, the disease is a chronic disease. The disease to be treated may be selected from psoriasis and inflammatory bowel disease. In further embodiments, the disease to be treated may be selected from psoriatic arthritis and Crohn's disease. Administering the high concentration 150 mg/ml liquid pharmaceutical formulation according to the present disclosure to the patient for therapy is advantageous for the reasons discussed herein.

### Further Embodiments of the 150 mg/ml Formulation

In the following, further specific contemplated embodiments of the 150 mg/ml antibody formulation according to the first aspect are disclosed:

1. A liquid pharmaceutical formulation comprising
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a polyol; and
c) a surfactant.

2. The formulation according to embodiment 1, comprising
d) a buffer.

3. The formulation according to embodiment 1 or 2, wherein the antibody is risankizumab.

4. The formulation according to any one of embodiments 1 to 3, wherein the antibody has been recombinantly produced in a mammalian cell.

5. The formulation according to embodiment 4, wherein the antibody has been recombinantly produced in a CHO cell.

6. The formulation according to one or more of embodiments 1 to 5, wherein the polyol is selected from a sugar, a sugar alcohol and combinations thereof.

7. The formulation according to embodiment 6, wherein the polyol is selected from trehalose, sucrose, sorbitol, mannitol and combinations thereof.

8. The formulation according to embodiment 6, wherein the polyol is a sugar, optionally selected from trehalose and sucrose.

9. The formulation according to embodiment 6, comprising trehalose as polyol.

10. The formulation according to embodiment 6, wherein the polyol is selected from sorbitol and mannitol.

11. The formulation according to embodiment 6, comprising mannitol as polyol.

12. The formulation according to any one of embodiments 1 to 11, wherein the liquid pharmaceutical formulation does not comprise sorbitol.

13. The formulation according to one or more of embodiments 1 to 9, wherein the formulation does not comprise a sugar alcohol.

14. The formulation according to one or more of embodiments 1 to 13, having one or more of the following characteristics:
(i) the concentration of the polyol in the formulation is at least 95 mM;
(ii) the concentration of the polyol in the formulation is at least 125 mM;
(iii) the concentration of the polyol in the formulation is at least 150 mM;
(iv) the concentration of the polyol in the formulation is ≤500 mM, ≤450 mM or ≤400 mM;
(v) the concentration of the polyol in the formulation is ≤350 mM, ≤300 mM or ≤275 mM; and/or
(vi) the concentration of the polyol in the formulation lies in the range of 95 mM to 450 mM or 125 mM to 400 mM;
optionally wherein the polyol is a sugar and/or a sugar alcohol.

15. The formulation according to one or more of embodiments 1 to 13, wherein the concentration of the polyol in the formulation is in the range of 95 mM to 250 mM, optionally wherein the polyol is a sugar.

16. The formulation according to one or more of embodiments 1 to 13, wherein the concentration of the polyol in the formulation is in the range of 125 mM to 225 mM, optionally wherein the polyol is a sugar such as trehalose.

17. The formulation according to one or more of embodiments 1 to 13, wherein the concentration of the polyol is in the range of 145 mM to 225 mM, optionally wherein the polyol is a sugar such as trehalose.

18. The formulation according to one or more of embodiments 1 to 13, wherein the polyol is a sugar and wherein the concentration of the sugar is in the range of 150 mM to 200 mM, optionally wherein the sugar is trehalose.

19. The formulation according to one or more of embodiments 1 to 13, wherein the polyol is a sugar and wherein the concentration of the sugar is in the range of 160 mM to 200 mM, optionally wherein the sugar is trehalose.

20. The formulation according to one or more of embodiments 1 to 13, wherein the polyol is a sugar and wherein the concentration of the sugar is in the range of 170 mM to 200 mM, optionally wherein the sugar is trehalose.

21. The formulation according to one or more of embodiments 1 to 13, comprising 185 mM trehalose as polyol.

22. The formulation according to one or more of embodiments 1 to 21, wherein the surfactant is a non-ionic surfactant.

23. The formulation according to embodiment 22, wherein the surfactant is a polysorbate.

24. The formulation according to embodiment 22 or 23, wherein the non-ionic surfactant is selected from polysorbate 20 and/or polysorbate 80.

25. The formulation according to one or more of embodiments 1 to 24, wherein the surfactant is polysorbate 20.

26. The formulation according to one or more of embodiments 1 to 25, in particular any one of embodiments 23 to 25, wherein the concentration of the surfactant in the formulation is at least 0.05 mg/ml, optionally at least 0.075 mg/ml.

27. The formulation according to one or more of embodiments 1 or 26, in particular any one of embodiments 23 to 25, wherein the concentration of the surfactant in the formulation is in a range of 0.05 mg/ml to 0.75 mg/ml.

28. The formulation according to one or more of embodiments 1 or 27, in particular any one of embodiments 23 to 25, wherein the concentration of the surfactant in the formulation is in the range of 0.05 mg/ml to 0.5 mg/ml or 0.075 mg/ml to 0.3 mg/ml.

29. The formulation according to embodiment 25, wherein the formulation comprises 0.2 mg/ml polysorbate 20 as surfactant.

30. The formulation according to embodiment 29, wherein the polyol is a sugar and wherein the concentration of the sugar is in the range of 145 mM to 225 mM, optionally wherein the sugar is trehalose.

31. The formulation according to one or more of embodiments 1 to 30, wherein the pH of the liquid pharmaceutical formulation is in the range of pH 5.0 to 7.5 or pH 5.0 to 7.0.

32. The formulation according to one or more of embodiments 1 to 30, wherein the pH of the liquid pharmaceutical formulation is in a range of 5.2 to 6.5 or 5.2 to 6.2.

33. The formulation according to one or more of embodiments 1 to 30, wherein the pH of the liquid pharmaceutical formulation is in a range of 5.5 to 6.5 or 5.5 to 6.2.

34. The formulation according to one or more of embodiments 1 to 30, wherein the pH of the liquid pharmaceutical formulation is in a range of 5.5 to 5.9.

35. The formulation according to one or more of embodiments 1 to 30, wherein the pH of the liquid pharmaceutical formulation is in a range of 5.6 to 5.8.

36. The formulation according to one or more of embodiments 1 to 30, wherein the pH of the liquid pharmaceutical formulation is 5.7 or 6.2.

37. The formulation according to one or more of embodiments 2 to 36, wherein the buffer has a pKa within 1.5 or one pH unit of the final pH of the liquid pharmaceutical formulation at 25° C., optionally wherein the buffer has a pKa within the range of pH 4.2 to 7.2 or pH 4.5 to 7 at 25° C.

38. The formulation according to one or more of embodiments 2 to 37, wherein the buffer is an organic buffer, which is optionally selected from an acetate buffer and a succinate buffer.

39. The formulation according to embodiment 38, wherein the buffer is an acetate buffer, optionally wherein the acetate buffer comprises sodium acetate and acetic acid.

40. The formulation according to one or more of embodiments 2 to 37, wherein the buffer is a histidine buffer or wherein the formulation fulfills at least one of the following characteristics: (i) it comprises a carboxylic acid buffer; (ii) it does not comprise a succinate buffer.

41. The formulation according to one or more of embodiments 2 to 40, comprising at least 1 mM, at least 2 mM or at least 3 mM buffer, optionally comprising at least 4 mM, at least 4.5 mM or at least 5 mM buffer.

42. The formulation according to one or more of embodiments 2 to 41, wherein the buffer concentration is 100 mM or less, 75 mM or less or 50 mM or less.

43. The formulation according to one or more of embodiments 2 to 41, wherein the buffer concentration is 20 mM or less or 15 mM or less.

44. The formulation according to one or more of embodiments 2 to 41, wherein the buffer concentration is in the range of 4 mM to 50 mM.

45. The formulation according to one or more of embodiments 2 to 41, wherein the buffer concentration is in the range of 5 mM to 25 mM or 5 mM to 20 mM.

46. The formulation according to one or more of embodiments 2 to 41, wherein the buffer concentration is in the range of 5 mM to 15 mM or 7 mM to 12 mM.

47. The formulation according to one or more of embodiments 2 to 41, wherein the buffer concentration is 10 mM.

48. The formulation according to one or more of embodiments 2 to 47, wherein the formulation comprises a single buffer, optionally an acetate buffer.

49. The formulation according to any one of embodiments 1 or 3 to 36, wherein the formulation does not comprise a buffer.

50. The formulation according to one or more of embodiments 1 to 49, wherein the formulation is an aqueous formulation.

51. The formulation according to any one of embodiments 2 to 48 or 50, comprising
a) 150 mg/ml of the antibody;
b) a sugar, optionally wherein the concentration of the sugar is in the range of 95 mM to 250 mM or 145 mM to 225 mM;
c) a non-ionic surfactant, optionally wherein the concentration of the non-ionic surfactant is in the range of 0.05 mg/ml to 0.5 mg/ml or 0.075 mg/ml to 0.3 mg/ml; and
d) a buffer.

52. The formulation according any one of embodiments 2 to 48 or 50 to 51, comprising
a) 150 mg/ml of the antibody;
b) trehalose, optionally wherein the concentration of trehalose is in the range of 95 mM to 250 mM or 145 mM to 225 mM;
c) a polysorbate, optionally wherein the concentration of the polysorbate is in the range of 0.05 mg/ml to 0.5 mg/ml or 0.075 mg/ml to 0.3 mg/ml; and
d) a buffer.

53. The formulation according to embodiment 51 or 52, wherein the buffer is an acetate buffer or a succinate buffer, optionally wherein the buffer concentration is in the range of 5 mM to 25 mM.

54. The formulation according to embodiment 52 or 53, wherein the polysorbate is polysorbate 20.

55. The formulation according to any one of embodiments 51 to 54, wherein the pH of the formulation is in a range of pH 5.2 to pH 6.5, optionally wherein the pH is in the range of 5.2 to 6.2 or 5.5 to 6.2 or is 5.7.

56. The formulation according any one of embodiments 2 to 48 or 50 to 55, comprising
a) 150 mg/ml of the antibody;
b) 170 mM to about 200 mM trehalose;
c) 0.1 mg/ml to 0.3 mg/ml polysorbate, optionally polysorbate 20; and
d) a buffer, optionally wherein the buffer is an acetate buffer.

57. The liquid pharmaceutical formulation according to one or more of embodiments 1, 3 to 36 or 49 to 50, comprising
a) 150 mg/ml of the antibody;
b) a polyol, optionally wherein the polyol is a sugar or sugar alcohol; and
c) a non-ionic surfactant, optionally a polysorbate;
d) no buffer.

58. The formulation according to embodiment 57, wherein the pH of the formulation is in a range of pH 5.2 to pH 6.5, optionally wherein the pH in the range of 5.2 to 6.2 or 5.5 to 6.2.

59. The formulation according to embodiment 58, wherein the pH is 5.7.

60. The formulation according to any one of embodiments 57 to 59, comprising 80 mM to 250 mM polyol, optionally wherein the polyol is trehalose.

61. The formulation according to any one of embodiments 57 to 60, wherein the concentration of the non-ionic surfactant is in the range of 0.05 mg/ml to 0.5 mg/ml, 0.075 mg/ml to 0.4 mg/ml or 0.1 mg/ml to 0.3 mg/ml.

62. The formulation according to embodiment 61, wherein the non-ionic surfactant is a polysorbate, optionally polysorbate 20.

63. The formulation according to one or more of embodiments 1 to 62, further comprising an amino acid as additive.

64. The formulation according to embodiment 63, wherein the amino acid has a charged side chain, optionally a positive-charged side chain such as L-arginine.

65. The formulation according to embodiment 63 or 64, wherein the amino acid is present in the formulation as a salt, optionally a hydrochloride (HCl) salt.

66. The formulation according to embodiment 63, wherein the amino acid is methionine.

67. The formulation according to embodiment 63, wherein the amino acid is L-proline.

68. The formulation according to one or more of embodiments 1 to 67, wherein the formulation has one or more of the following characteristics
(i) it does not comprise arginine;
(ii) it does not comprise an amino acid with a positive-charged side chain;
(iii) it does not comprise an amino acid with a charged side chain;
(iv) it does not comprise methionine; and/or
(v) it does not comprise an amino acid as additive.

69. The liquid pharmaceutical formulation according to any one of embodiments 2 to 68, comprising
a) 150 mg/ml of the antibody;
b) 185 mM trehalose;
c) 0.2 mg/ml polysorbate 20; and
d) 10 mM acetate buffer;
wherein the pH is in the range of 5.2 to 6.2 and optionally is 5.7.

70. The formulation according to any one of embodiments 1 to 69, wherein the formulation is stable.

71. The formulation according to embodiment 70, fulfilling one or more of the following stability characteristics:
(i) following storage at 5° C. for 36 months, at least 94%, at least 95% or at least 96% of the antibody is present as a monomer as measured by UP-SEC, and/or the relative monomer content of the antibody does not decrease by more than 3%, more than 2.5%, more than 2% or more than 1.5%;
(ii) following storage at 5° C. for 24 months, at least 94%, at least 95% or at least 96% of the antibody is present as a monomer as measured by UP-SEC, and/or the relative monomer content of the antibody does not decrease by more than 3%, more than 2.5%, more than 2%, more than 1.5% or more than 1%;
(iii) following storage at 5° C. for 9 months, at least 96% or at least 96.5% of the antibody is present as a monomer as measured by UP-SEC, and/or the relative monomer content of the antibody does not decrease by more than 1.5% or more than 1%;
(iv) following storage at 5° C. for 3 months, at least 96% or at least 97% of the antibody is present as a monomer as measured by UP-SEC, and/or the relative monomer content of the antibody does not decrease by more than 1% or more than 0.7% or more than 0.5%;
(v) following storage at 25° C. for 12 months, at least 90% or at least 92% of the antibody is present as a monomer as measured by UP-SEC, and/or the relative monomer content of the antibody does not decrease by more than 7% or more than 6% or more than 5%;
(vi) following storage at 25° C. for 3 months, at least 95% of the antibody is present as a monomer as measured by UP-SEC, and/or the relative monomer content of the antibody does not decrease by more than 3% or more than 2%;
(vii) following storage at 25° C. for 1 month, at least 96% of the antibody is present as a monomer as measured by UP-SEC, and/or the relative monomer content of the antibody does not decrease by more than 2% or more than 1%;
(viii) following storage at 40° C. for 3 months, at least 87% or at least 88% of the antibody is present as a monomer as measured by UP-SEC, and/or the relative monomer content of the antibody does not decrease by more than 10% or more than 9% or more than 8%; and/or
(ix) following storage at 40° C. for 1 month, at least 93% or at least 94% of the antibody is present as a monomer as measured by UP-SEC, and/or the relative monomer content of the antibody does not decrease by more than 5% or more than 4%.

72. The formulation according to embodiment 70 or 71, fulfilling one or more of the following stability characteristics:
(i) following storage at 5° C. for at least 36 months, the formulation has an opalescence of 12 FNU (Formazin Nephelometry Units) or less or 10 FNU or less, and/or the opalescence does not increase by more than 5 FNU or more than 3 FNU;
(ii) following storage at 5° C. for at least 3, 6, 9, 12, 18 or 24 months, the formulation has an opalescence of 12 FNU (Formazin Nephelometry Units) or less or 10 FNU or less, and/or the opalescence does not increase by more than 5 FNU or more than 3 FNU;
(iii) following storage at 25° C. for at least 1, 3, 6, 9 or 12 months, the formulation has an opalescence of 12 FNU or less or 10 FNU or less, and/or the opalescence does not increase by more than 7 FNU or more than 5 FNU;
(iv) following storage at 40° C. for at least 1 or 3 months, the formulation has an opalescence of 12 FNU or less or 10 FNU or less, and/or the opalescence does not increase by more than 5 FNU or more than 3 FNU; and/or
(v) following shaking at 25° C. for 21 days, the formulation has an opalescence of 12 FNU or less or 10 FNU or less, and/or the opalescence of the formulation does not increase by more than 3 FNU or more than 2 FNU.

73. The formulation according to any one of embodiments 70 to 72, fulfilling one or both of the following stability characteristics:
(i) following shaking at 25° C. for 21 days, at least 95% or at least 96% of the antibody is present as a monomer as measured by UP-SEC, and/or the relative monomer content of the antibody does not decrease by more than 2% or more than 1%; and/or
(ii) following shaking at 25° C. for 21 days, less than 3% or less than 2% of the antibody is present as high molecular weight (HMW) species as measured by UP-SEC, and/or the relative HMW content of the antibody does not increase by more than 2% or more than 1.5% or more than 1%.

74. The formulation according to any one of embodiments 70 to 73, fulfilling one or more of the following stability characteristics:
(i) following storage at 5° C. for 36 months, less than 4% or less than 3% of the antibody is present as high molecular weight (HMW) species as measured by UP-SEC, and/or the relative HMWS content of the antibody does not increase by more than 2% or more than 1.5%;
(ii) following storage at 5° C. for 24 months, less than 4% or less than 3% of the antibody is present as high molecular weight (HMW) species as measured by UP-SEC, and/or the relative HMW content of the antibody does not increase by more than 2% or more than 1.5% or more than 1%;
(iii) following storage at 5° C. for 9 months, less than 4% or less than 3% or less than 2.5% of the antibody is present as high molecular weight (HMW) species as measured by UP-SEC, and/or the relative HMW content of the antibody does not increase by more than 1% or more than 0.8% or more than 0.6%;
(iv) following storage at 5° C. for 3 months, less than 4% or less than 3% or less than 2.5% of the antibody is present as high molecular weight (HMW) species as measured by UP-SEC, and/or the relative HMW content of the antibody does not increase by more than 1% or more than 0.8% or more than 0.6%;
(v) following storage at 40° C. for 3 months, less than 6.5% or less than 6% or less than 5.5% of the antibody is present as a high molecular weight (HMW) species as measured by UP-SEC, and/or the relative HMW content of the antibody does not increase by more than 5% or more than 4%; and/or
(vi) following storage at 40° C. for 1 month, less than 5% or less than 4.5% or less than 4% of the antibody is present as high molecular weight (HMW) species as measured by UP-SEC, and/or the relative HMW content of the antibody does not increase by more than 2.5% or more than 2%.

75. The formulation according to any one of embodiments 70 to 74, fulfilling one or more of the following stability characteristics:
(i) following storage at 25° C. for 12 months, less than 5% or less than 4% of the antibody is present as high molecular weight (HMW) species as measured by UP-SEC, and/or the relative HMW content of the antibody does not increase by more than 3% or more than 2.5% or more than 2%;
(ii) following storage at 25° C. for 3 months, less than 4% or less than 3.5% or less than 3.2% of the antibody is present as high molecular weight (HMW) species as measured by UP-SEC, and/or the relative HMW content of the antibody does not increase by more than 2% or more than 1.5%; and/or
(iii) following storage at 25° C. for 1 month, less than 4% or less than 3.5% or less than 3% of the antibody is present as high molecular weight (HMW) species as measured by UP-SEC, and/or the relative HMW content of the antibody does not increase by more than 1.5% or more than 1%.

76. The formulation according to any one of embodiments 70 to 75, fulfilling one or more of the following stability characteristics:
(i) following storage at 5° C. for 36 months, less than 2% or less than 1.5% of the antibody is present as low molecular weight (LMW) species as measured by UP-SEC, and/or the relative LMWS content of the antibody does not increase by more than 1.5% or more than 1.5% or more than 0.5%;
(ii) following storage at 5° C. for 24 months, less than 2% or less than 1.5% of the antibody is present as low molecular weight (LMW) species as measured by UP-SEC, and/or the relative LMW content of the antibody does not increase by more than 1.5% or more than 1.5% or more than 0.5%;
(iii) following storage at 5° C. for 9 months, less than 2% or less than 1.5% of the antibody is present as a low molecular weight (LMW) species as measured by UP-SEC, and/or the relative LMW content of the antibody does not increase by more than 1.5% or more than 1.5% or more than 0.5%;
(iv) following storage at 5° C. for 3 months, less than 2% or less than 1.5% or less than 1% of the antibody is present as a low molecular weight (LMW) species as measured by UP-SEC, and/or the relative LMW content of the antibody does not increase by more than 1% or more than 0.5% or more than 0.25%;
(v) following storage at 40° C. for 3 months, less than 8% or less than 7% or less than 6% of the antibody is present as a low molecular weight (LMW) species as measured by UP-SEC, and/or the relative LMW content of the antibody does not increase by more than 8% or more than 7% or more than 6%; and/or
(vi) following storage at 40° C. for 1 month, less than 4% or less than 3.5% or less than 3% of the antibody is present as a low molecular weight (LMW) species as measured by UP-SEC, and/or the relative LMW content of the antibody does not increase by more than 3% or more than 2.5% or more than 2.2%.

77. The formulation according to any one of embodiments 70 to 76, fulfilling one or more of the following stability characteristics:
(i) following storage at 25° C. for 12 months, less than 6% or less than 5% or less than 4.5% of the antibody is present as a low molecular weight (LMW) species as measured by UP-SEC, and/or the relative LMW content of the antibody does not increase by more than 5% or more than 4% or more than 3%;
(ii) following storage at 25° C. for 3 months, less than 3% or less than 2% or less than 1.8% of the antibody is present as a low molecular weight (LMW) species as measured by UP-SEC, and/or the relative LMW content of the antibody does not increase by more than 2% or more than 1.5% or more than 1%; and/or
(iii) following storage at 25° C. for 1 month, less than 2% or less than 1.5% or less than 1.2% of the antibody is present as a low molecular weight (LMW) species as measured by UP-SEC, and/or the relative LMW content of the antibody does not increase by more than 1% or more than 0.6% or more than 0.4%.

78. The formulation according to any one of embodiments 70 to 77, fulfilling one or more of the following stability characteristics:
(i) following storage at 5° C. for 36 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC);
(ii) following storage at 5° C. for 24 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC);
(iii) following storage at 5° C. for 6 months, at least 60% or at least 65% of the antibody is present as main peak variants, and/or the relative content of main peak variants of the antibody does not decrease by more than 5% or more than 4%, as determined by ion exchange chromatography (IEC);
(iv) following storage at 5° C. for 3 months, at least 60% or at least 65% of the antibody is present as main peak variants, and/or the relative content of main peak variants of the antibody does not decrease by more than 4% or more than 3% or more than 2%, as determined by ion exchange chromatography (IEC);
(v) following storage at 25° C. for 12 months, at least 35% or at least 40% or at least 45% of the antibody is present as main peak variants, and/or the relative content of main peak variants of the antibody does not decrease by more than 35% or more than 30% or more than 25%, as determined by ion exchange chromatography (IEC);
(vi) following storage at 25° C. for 3 months, at least 55% or at least 60% of the antibody is present as main peak variants, and/or the relative content of main peak variants of the antibody does not decrease by more than 15% or more than 10%, as determined by ion exchange chromatography (IEC); and/or
(vii) following storage at 25° C. for 1 month, at least 60% or at least 65% of the is present as main peak variants, and/or the relative content of main peak variants of the antibody does not decrease by more than 10% or more than 5%, as determined by ion exchange chromatography (IEC).

79. The formulation according to any one of embodiments 70 to 78, fulfilling one or more of the following stability characteristics:
(i) following storage at 5° C. for 36 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(ii) following storage at 5° C. for 24 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(iii) following storage at 5° C. for 6 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(iv) following storage at 5° C. for 3 months, less than 30% or less than 28% of the antibody is present as acidic peak group variants, and/or the relative content of acidic peak group variants of the antibody does not increase by more than 3% or more than 2% or more than 1%, as determined by ion exchange chromatography (IEC);
(v) following storage at 25° C. for 12 months, less than 50%, less than 45% or less than 40% of the antibody is present as acidic peak group variants, and/or the relative content of acidic peak group variants of the antibody does not increase by more than 30% or more than 25% or more than 20%, as determined by ion exchange chromatography (IEC);
(vi) following storage at 25° C. for 3 months, less than 40% or less than 35% or less than 30% of the antibody is present as acidic peak group variants, and/or the relative content of acidic peak group variants of the antibody does not increase by more than 10% or more than 8% or more than 6%, as determined by ion exchange chromatography (IEC); and/or
(vii) following storage at 25° C. for 1 month, less than 35% or less than 30% or less than 28% of the antibody is present as acidic peak group variants, and/or the relative content of acidic peak group variants of the antibody does not increase by more than 4% or more than 3%, as determined by ion exchange chromatography (IEC).

80. The formulation according to any one of embodiments 70 to 79, fulfilling one or more of the following stability characteristics:
(i) following storage at 5° C. for 36 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC);
(ii) following storage at 5° C. for 24 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC);
(iii) following storage at 5° C. for 6 months, less than 15% or less than 10% of the antibody is present as basic peak group variants, and/or the relative content of basic peak group variants of the antibody does not increase by more than 4% or more than 3% or more than 2%, as determined by ion exchange chromatography (IEC);
(iv) following storage at 5° C. for 3 months, less than 15% or less than 10% of the antibody is present as basic peak group variants, and/or the relative content of basic peak group variants of the antibody does not increase by more than 3% or more than 2%, as determined by ion exchange chromatography (IEC);
(v) following storage at 25° C. for 12 months, less than 30% or less than 25% or less than 22% of the antibody is present as basic peak group variants, and/or the relative content of basic peak group variants of the antibody does not increase by more than 25% or more than 20% or more than 15%, as determined by ion exchange chromatography (IEC);
(vi) following storage at 25° C. for 3 months, less than 20% or less than 15% or less than 12% of the antibody is present as basic peak group variants, and/or the relative content of basic peak group variants of the antibody does not increase by more than 9% or more than 7% or more than 5%, as determined by ion exchange chromatography (IEC); and/or
(vii) following storage at 25° C. for 1 month, less than 15% or less than 10% or less than 9% of the antibody is present as basic peak group variants, and/or the relative content of basic peak group variants of the antibody does not increase by more than 3% or more than 2%, as determined by ion exchange chromatography (IEC).

81. The formulation according to any one of embodiments 70 to 80, fulfilling one or more of the following stability characteristics
(i) following storage at 5° C. for 36 months, at least 95% or at least 97% of the specific binding activity to IL-23 is measured compared to a reference antibody, wherein the reference antibody has not been stored;
(ii) following storage at 5° C. for 4, 6, 9, 12, 18 or 24 months, at least 95% or at least 97% of the specific binding activity to IL-23 is measured compared to a reference antibody, wherein the reference antibody has not been stored;
(iii) following storage at 25° C. for 2, 3, 4, 6, 9, 12, or 18 months, at least 93% or at least 96% of the specific binding activity to IL-23 is measured compared to a reference antibody, wherein the reference antibody has not been stored; and/or
(iv) following storage at 40° C. for 3, 4 or 6 months, at least 90% or at least 95% of the specific binding activity to IL-23 is measured compared to a reference antibody, wherein the reference antibody has not been stored.

82. The formulation according to one or more of embodiments 1 to 81, wherein the dynamic viscosity measured at 20° C. is ≤30 mPas (mPa s), ≤25 mPas or ≤20 mPas.

83. The formulation according to one or more of embodiments 1 to 82, wherein the formulation has a conductivity in a range of 0.8 to 5 mS/cm, optionally in a range of 1 to 2 mS/cm or 1.2 to 1.8 mS/cm.

84. The formulation according to one or more of embodiments 1 to 83, wherein the formulation has an osmolality in a range of 225 mOsm/kg to 375 mOsm/kg, such as 250 mOsm/kg to 350 mOsm/kg, 275 mOsm/kg to 330 mOsm/kg or 290 mOsm/kg to 320 mOsm/kg.

85. The formulation according to one or more of embodiments 1 to 84, wherein the formulation is an injectable formulation.

86. The formulation according to embodiment 85, wherein the formulation is suitable for subcutaneous injection.

87. The formulation according to one or more of embodiments 1 to 86, wherein the formulation is not and has not been subjected to a reconstitution step before administration.

88. The formulation according to any one of embodiments 1 to 86, prepared by reconstitution from a lyophilized formulation.

89. A lyophilized formulation of an anti-IL-23p19 antibody, wherein the antibody comprises a light chain amino acid sequence according to SEQ ID NO: 1 and a heavy chain amino acid sequence according to SEQ ID NO: 2, made by lyophilizing the liquid formulation as defined in any one of embodiments 1 to 69, optionally wherein the liquid formulation is an aqueous solution.

90. A lyophilized formulation of an anti-IL-23p19 antibody, wherein the antibody comprises a light chain amino acid sequence according to SEQ ID NO: 1 and a heavy chain amino acid sequence according to SEQ ID NO: 2, providing upon reconstitution the liquid formulation as defined in any one of embodiments 1 to 69 or 82 to 86.

91. A lyophilized formulation comprising
a) an anti-IL-23p19 antibody, wherein the antibody comprises a light chain amino acid sequence according to SEQ ID NO: 1 and a heavy chain amino acid sequence according to SEQ ID NO: 2, in an amount that upon reconstitution provides an antibody concentration of 150 mg/ml;
b) a polyol;
c) a surfactant; and
d) optionally a buffer.

92. The lyophilized formulation according to embodiment 91, wherein the antibody is risankizumab.

93. The lyophilized formulation according to embodiment 91 or 92, wherein the polyol has one or more of the characteristics as defined in any one of embodiments 6 to 13, optionally wherein the polyol is a sugar, optionally selected from trehalose and sucrose.

94. The lyophilized formulation according to any one of embodiments 91 to 93, wherein the surfactant has one or more of the characteristics as defined in any one of embodiments 22 to 25, optionally wherein the surfactant is a polysorbate.

95. The lyophilized formulation according to any one of embodiments 91 to 94, comprising d) a buffer, wherein the buffer has one or more of the characteristics as defined in any one of embodiments 37 to 40.

96. The lyophilized formulation according to any one of embodiments 91 to 95, wherein the formulation has a pH as defined in any one of embodiments 31 to 36 when reconstituted.

97. A sealed container, optionally, a vial or pre-filled syringe, containing the liquid pharmaceutical formulation according to any one of embodiments 1 to 87.

98. A container, optionally, a sealed vial, containing the lyophilized formulation according to any one of embodiments 88 to 96.

99. The product according to embodiment 97, wherein the container contains 2 ml or less of the liquid formulation, optionally 1.5 ml or less or 1 ml or less of the liquid formulation.

100. The product according to any one of embodiments 97 to 99, comprising a single dose of 150 mg antibody.

101. The formulation according to any one of embodiments 1 to 96 or the product according to any one of embodiments 97 to 100 for therapeutic treatment of a human subject.

102. The formulation according to any one of embodiments 1 to 96 or the product according to any one of embodiments 97 to 100 for use in the treatment of a disease selected from psoriasis and inflammatory bowel disease.

103. The formulation according to any one of embodiments 1 to 96 or the product according to any one of embodiments 97 to 100 for use in the treatment of a disease selected from psoriatic arthritis and Crohn's disease.

104. A stable liquid pharmaceutical formulation comprising
a) 150 mg/ml of an anti-IL-23p19 antibody, wherein the antibody comprises a light chain amino acid sequence according to SEQ ID NO: 1 and a heavy chain amino acid sequence according to SEQ ID NO: 2;
b) a tonicity modifier; and
c) a surfactant, wherein the formulation has a pH of 5.5-5.9 and the formulation is isotonic.

105. The stable formulation according to embodiment 104, wherein the antibody is risankizumab.

106. The stable formulation according to embodiment 104 or 105, wherein tonicity modifier is a polyol.

107. The stable formulation according to any one of embodiments 104 to 106, wherein the tonicity modifier is a polyol as defined in any one of the above embodiments 6 to 11.

108. The stable formulation according to any one of embodiments 104 to 107, wherein the concentration of the tonicity modifier has one or more of the characteristics as defined for the polyol in any one of the above embodiments 14 to 21, optionally wherein the tonicity modifier is a polyol as defined therein, optionally a sugar and/or sugar alcohol, optionally selected from trehalose and sucrose.

109. The stable formulation according to any one of embodiments 104 to 108, wherein the surfactant has one or more of the characteristics as defined in any one of the above embodiments 22 to 25, optionally wherein the surfactant is a polysorbate, optionally selected from polysorbate 20 and polysorbate 80.

110. The stable formulation according to any one of embodiments 104 to 109, wherein the concentration of the surfactant in the formulation is as defined in any one of the above embodiments 26 to 30.

111. The stable formulation according to any one of embodiments 104 to 110, wherein the formulation has a pH of in the range of 5.6 to 5.8, optionally wherein the pH of the formulation is 5.7.

112. The stable formulation according to any one of embodiments 104 to 111, comprising d) a buffer, optionally wherein the buffer has one or more of the characteristics as defined in any one of embodiments 37 to 40 and 48.

113. The stable formulation according to embodiment 112, wherein the buffer has a concentration as defined in any one of embodiments 41 to 47.

114. The stable formulation according to any one of embodiments 104 to 113, wherein the formulation is an aqueous formulation.

115. The stable formulation according to any one of embodiments 104 to 114, fulfilling one or more of the stability characteristics as defined in any one of embodiments 71 to 81.

116. The stable formulation according to any one of embodiments 104 to 111 or 114 to 115, wherein the formulation is buffer-free.

117. The stable formulation according to any one of embodiments 104 to 116, wherein the formulation has an osmolality of 290-320 mOsm/kg.

118. The stable formulation according to any one of embodiments 104 to 117, having at least one or at least two of the following characteristics:
(i) the surfactant is a non-ionic surfactant;
(ii) the surfactant is a polysorbate, optionally selected from polysorbate 20 and polysorbate 80;
(iii) wherein the concentration of the surfactant in the formulation is in a range of 0.05 mg/ml to 0.5 mg/ml, optionally within a range of 0.075 mg/ml to 0.4 mg/ml or 0.1 mg/ml to 0.3 mg/ml; and/or
(iv) it has any one of the characteristics as defined in embodiments 12, 13, or 63 to 68.

119. The stable formulation according to any one of embodiments 104 to 118, prepared by reconstitution from a lyophilized formulation.

120. A lyophilized formulation of an anti-IL-23p19 antibody, wherein the antibody comprises a light chain amino acid sequence according to SEQ ID NO: 1 and a heavy chain amino acid sequence according to SEQ ID NO: 2, made by lyophilizing the liquid formulation as defined in any one of embodiments 104 to 118, optionally wherein the stable liquid formulation is an aqueous solution.

121. A lyophilized formulation of an anti-IL-23p19 antibody, wherein the antibody comprises a light chain amino acid sequence according to SEQ ID NO: 1 and a heavy chain amino acid sequence according to SEQ ID NO: 2, providing upon reconstitution the stable liquid formulation as defined in any one of embodiments 104 to 118.

Numeric ranges are inclusive of the numbers defining the range. The headings provided herein are not limitations of the various aspects or embodiments of this disclosure which can be read by reference to the specification as a whole.

As used in the subject specification, the singular forms “a”, “an” and “the” include plural referents unless the context clearly dictates otherwise. The terms “include,” “have,” “comprise” and their variants are used synonymously and to be construed as non-limiting.

Throughout the specification, where compositions are described as comprising components or materials, it is contemplated that the compositions can in embodiments also consist essentially of, or consist of, any combination of the recited components or materials, unless described otherwise. The technology illustratively disclosed herein suitably may be practiced in the absence of any element which is not specifically disclosed herein.

### EXAMPLES

The following examples are for illustrative purposes only and are not to be construed as limiting this invention in any manner.

### I. Material and Methods

### 1. Preparation of the Starting Material

The starting material of risankizumab, produced in CHO cells and purified, was adjusted to pH 5.9, where necessary, prior to UF/DF process. At the end, the solution was concentrated and the concentrated starting material was used for preparing formulations according to the subsequent examples.

### 2. Syringes

Storage of formulations in syringes was essentially performed by using Neopak syringes with rubber stopper of Becton Dickinson (USA). The break loose force, as well as the maximum and average gliding force were measured using such syringes. In embodiments, 1 ml Neopak syringes with a 27 gauge 12 inch needle and a rubber stopper of Becton Dickinson (USA) were used.

### II. Example 1: Characterization of the Starting Material

### 1. Influence of the pH on the Antibody

RALS (right-angle light scattering) measurements were performed for risankizumab at different pH values in a buffer mixture of 10 mM acetate, 10 mM citrate, 10 mM phosphate, 115 mM NaCl. The results are shown in Table 1.

TABLE 1
Results of the RALS measurements for different pH values.

| pH value | | 4.5 | 5.0 | 5.5 | 6.0 | 6.5 | 7.0 |
|---|---|---|---|---|---|---|---|
| RALS | Tₒₙₛₑₜ/° C. | 70/71 | 72/72 | 75/75 | 76/76 | 76/75 | 76/76 |

The results show that the onset of unfolding increases when increasing the pH until a plateau is reached. The highest onset temperatures indicating high stability were measured above pH of 5.0 to 7. Therefore, the pH should not be too acidic (<5).

### 2. Determination of the Buffering Capacity of the Starting Material

The buffer capacity of the antibody starting material was determined inter alia in order to facilitate the pH adjustment of the formulation solutions of the subsequent stability studies and to avoid protein damage. The titration to determine the buffer capacity of the antibody was performed at the following concentrations: 150 mg/mL; 100 mg/mL; 50 mg/mL (twice) and 20 mg/mL.

The dilution was carried out in beakers according to Table 2.

TABLE 2
Dilution scheme for determination of the buffering
capacity of the starting material.

| Target | Volume of | Volume | Final |
|---|---|---|---|
| concentration | initial solution | MilliQ | volume |
| [mg/mL] | [mL] | [mL] | [mL] |
| 150 | 4.67 | 1.33 | 5.00 |
| 100 | 3.11 | 2.89 | 5.00 |
| 50 | 1.56 | 4.44 | 5.00 |
| 50 | 2.6 | 7.40 | 10.00 |

After dilution, 5 mL of the respective solution was transferred into a 10R glass vial using a volumetric pipette and titrated. 0.2 M NaOH solution was selected for titration (stirring speed 250 rpm). For each antibody concentration the titration was performed and the amount of added NaOH was calculated. The slope and the reciprocal value of the slope as shown in Table 3 were calculated using Excel.

TABLE 3
Results of the calculation of the slope and the
corresponding reciprocal values.

| Protein concentration | | Reciprocal |
|---|---|---|
| (mg/mL) | Slope | of the slope |
| 20.47 | 1.090000 | 0.92 |
| 50.74 | 0.434821 | 2.30 |
| 50.77 | 0.432378 | 2.31 |
| 102.33 | 0.201703 | 4.96 |
| 152.30 | 0.133120 | 7.51 |

The concentration dependent buffer capacity levels were obtained by drawing the concentration against the reciprocal values of the slope. As a result, a straight line with a slope of 0.0505 and a y-axis intercept of −0.2007 was obtained. The results and the below examples show that risankizumab itself has significant buffering capacity allowing to prepare 150 mg/ml buffer-free formulations according to the present disclosure that do not comprise any further/additional buffer substance.

### III. Example 2: Analysis of Different pH Values and Buffer Substances

1. Comparison of Acetate and Succinate Buffer Systems with Different pH Values for the Assessment of Stability of 150 mg/mL Formulations

### 1.1. Preparation of Formulations

The formulations shown in Table 4 were prepared and analyzed:

TABLE 4
Composition of the analyzed formulations.

| | | Protein | | | | | |
|---|---|---|---|---|---|---|---|
| | | concentration | | | | | |
| Formulation | pH | mg/mL | buffer | | Excipient 1 | Excipient 2 | Excipient 3 |
| F1 | 6.2 | 150 | 4.4 | mM | 200 mM | — | 0.02% PS20 |
| | | | succinate | | sorbitol | | |
| F2 | 5.9 | 150 | 4.4 | mM | 200 mM | — | 0.02% PS20 |
| | | | succinate | | sorbitol | | |
| F3 | 5.7 | 150 | 10 | mM | 190 mM | — | 0.02% PS20 |
| | | | acetate | | sorbitol | | |
| F4 | 5.7 | 150 | 10 | mM | 115 mM | 50 mM L-arginine | 0.02% PS20 |
| | | | acetate | | sorbitol | HC1 | |
| F5 | 5.7 | 150 | 10 | mM | 150 mM | 50 mM L-proline | 0.02% PS20 |

| | |
|---|---|
| acetate | sorbitol |

0.02% PS20 corresponds to 0.2 mg/mL PS20.

Samples were taken within a period of 18 months (0, 3, 6, 8, 12 and 18 months). Storage conditions were: 5° C. and 25° C./60% relative humidity. Each formulation was filled into Neopak syringes. The filling within a laminar flow was set to a volume of 1.1 mL. The syringes were closed with a stopper and analyzed visually for particles before storage. Afterwards, the syringes were stored hanging in syringe-trays at the respective temperature. In parallel, the buffer solutions were stored as controls. After preparing the formulations, each solution was sterile filtered and the prepared formulations were stored in syringes (Neopak syringes of Becton Dickinson (USA).

### 1.2. Analytics

For analysis of the samples, inter alia high pressure size exclusion chromatography (HP-SEC) and ultra-performance size exclusion chromatography (UP-SEC) were performed and the turbidity at 860 nm (also referred to as opalescence) was measured. The syringeability of the formulations stored in syringes (Neopak) was analyzed by measuring the mechanical forces that are required to release/inject the formulation. A pressure test was performed with a speed of 379.2 mm/min (5 sec). The viscosity of the formulations was measured at 20° C. using the HAAKE RheoStress 600 with a C35/1 rotor. Double measurements were performed.

Further details on the utilized analysis methods are described below.

### 1.3. Results

### 1.3.1. Measurement of the Monomer Content

For determining the monomer content the samples were analyzed by UP-SEC and UP-SEC.

### HP-SEC Analysis

The results obtained by HP-SEC analysis at a storage temperature of 25° C. and 5° C. are shown in Table 5:

TABLE 5
Results HP-SEC monomer in % analysis over 18 months
storage at different temperatures in syringes.

| | Temp- | F1 | F2 | F3 | F4 | F5 |
|---|---|---|---|---|---|---|
| Time | erature | in % | in % | in % | in % | in % |
| Initial | 25° C. | 95.8 | 96.4 | 96.6 | 96.7 | 96.5 |
| 3 months | | 94.5 | 95.1 | 95.4 | 96.1 | 95.6 |
| 6 months | | 94.4 | 94.9 | 95.1 | 95.8 | 95.4 |
| 8 months | | 93.7 | 94.3 | 94.6 | 95.3 | 94.9 |
| 12 months | | 92.9 | 93.5 | 93.9 | 94.8 | 94.3 |
| 18 months | | 91.1 | 92.4 | 92.8 | 94.0 | 93.4 |
| Initial | 5° C. | 95.8 | 96.4 | 96.6 | 96.7 | 96.5 |
| 6 months | | 95.6 | 96.2 | 96.4 | 96.6 | 96.4 |
| 8 months | | 95.2 | 95.9 | 96.1 | 96.4 | 96.2 |
| 18 months | | 93.4 | 94.7 | 95.1 | 95.8 | 95.4 |

Over a storage time of 18 months at 25° C. the monomer content was between 91-94%. The strongest decrease was measured for F1 with −5%; the lowest decrease was measured with −3% for formulation 4 and 9 tested in this example. Over a storage time of 18 months at 5° C. the monomer content was between 9396%. The strongest decrease was measured for F with −2.4%; the lowest decrease was measured with −0.9% and −1.1% for formulation 4 and 5.

### UP-SEC Analysis

The results of the UP-SEC analysis were analogous to those of the UP-SEC and thus confirm these results.

TABLE 5a
Results UP-SEC monomer in % analysis over 18 months
storage at different temperatures in syringes.

| | Temp- | F1 | F2 | F3 | F4 | F5 |
|---|---|---|---|---|---|---|
| Time | erature | in % | in % | in % | in % | in % |
| Initial | 25° C. | 94.9 | 95.4 | 95.5 | 95.7 | 95.4 |
| 3 months | | 93.5 | 94.2 | 94.4 | 95.0 | 94.6 |
| 8 months | | 93.6 | 92.7 | 93.0 | 93.6 | 93.3 |
| 12 months | | 90.6 | 91.2 | 91.4 | 92.1 | 91.7 |
| 18 months | | 88.4 | 89.4 | 89.8 | 90.6 | 90.3 |
| Initial | 5° C. | 94.9 | 95.4 | 95.5 | 95.7 | 95.4 |
| 8 months | | 94.6 | 95.3 | 95.5 | 95.7 | 95.7 |
| 18 months | | 92.7 | 93.8 | 94.2 | 94.7 | 94.4 |

Over a storage time of 18 months at 25° C. the monomer content (UP-SEC) was between 88-91%. The strongest decrease was measured for F1 with −6.4%; the lowest decrease was measured with −5.1% and −5.4% for formulation 4 and 5. Over a storage time of 18 months at 5° C. the monomer content (UP-SEC) was between 93-95%. The strongest decrease was measured for F1 with −2.2%; the lowest decrease was measured with −1% for formulation 4 and 5.

### Results and Discussion

All tested formulations were overall stable in view of the monomer content, showing that these formulations are stable at 5° C. and 25° C. over long storage times up to 18 months.

### 1.3.2. Measurement of the HMW Content

For determining the HMW content the samples were analyzed by HP-SEC and UP-SEC.

### HP-SEC Analysis

The results obtained by HP-SEC analysis at a storage temperature of 25° C. and 5° C. are shown in Table 6.

TABLE 6
Results HP-SEC HMW content in % at different storage
temperatures over 18 months in syringes.

| | Temp- | F1 | F2 | F3 | F4 | F5 |
|---|---|---|---|---|---|---|
| Time | erature | in % | in % | in % | in % | in % |
| Initial | 25° C | 4.0 | 3.4 | 3.2 | 3.1 | 3.3 |
| 3 months | | 5.1 | 4.5 | 4.3 | 3.6 | 4.0 |
| 6 months | | 5.1 | 4.6 | 4.4 | 3.6 | 4.1 |
| 8 months | | 5.7 | 5.1 | 4.8 | 4.0 | 4.5 |
| 12 months | | 6.2 | 5.5 | 5.2 | 4.2 | 4.8 |
| 18 months | | 7.6 | 6.4 | 5.9 | 4.7 | 5.3 |
| Initial | 5° C. | 4.0 | 3.4 | 3.2 | 3.1 | 3.3 |
| 6 months | | 4.2 | 3.6 | 3.4 | 3.2 | 3.4 |
| 8 months | | 4.6 | 3.9 | 3.7 | 3.4 | 3.7 |
| 18 months | | 6.2 | 5.0 | 4.5 | 3.9 | 4.3 |

Over a storage time of 18 months at 25° C. the HMW species were increased by 2-4%. The strongest increase was measured for F1 with +3.6%; the lowest increase was measured with +1.6% and +2.0% for formulation 4 and 5. Over a storage time of 18 months at 5° C. the HMW species were increased by 1-2%. The strongest increase was measured for F1 with +2.2%; the lowest increase was measured with +0.8% and +1.0% for formulation 4 and 5.

### UP-SEC Analysis

The results of the UP-SEC analysis were analogous to those of the HP-SEC and thus confirm these results.

TABLE 6a
Results UP-SEC BMW content in % at different storage
temperatures over 18 months in syringes.

| | Tem- | F1 | F2 | F3 | F4 | F5 |
|---|---|---|---|---|---|---|
| Time | perature | in % | in % | in % | in % | in % |
| Initial | 25° C. | 3.9 | 3.4 | 3.2 | 3.1 | 3.3 |
| 3 months | | 5.1 | 4.5 | 4.2 | 3.6 | 3.9 |
| 8 months | | 4.0 | 5.0 | 4.8 | 4.0 | 4.4 |
| 12 months | | 6.0 | 5.4 | 5.0 | 4.2 | 4.7 |
| 18 months | | 7.4 | 6.4 | 6.0 | 4.9 | 5.5 |
| Initial | 5° C. | 3.9 | 3.4 | 3.2 | 3.1 | 3.3 |
| 8 months | | 4.4 | 3.7 | 3.5 | 3.3 | 3.5 |
| 18 months | | 5.8 | 4.7 | 4.3 | 3.8 | 4.1 |

Over a storage time of 18 months at 25° C. the FNM were increased by 2-3%. The strongest increase was measured for F1 with +3.5%; the lowest increase was measured with +1.8% and +2.2% for formulation 4 and 5. Over a storage time of 18 months at SOC the HMW were increased by 0.7-1.9%. The strongest increase was measured for F1 with +1.9%; the lowest increase was measured with +0.7% and +0.8% for formulation 4 and 5.

### Results and Discussion

All tested formulations were overall stable in view of the HMW content, showing that various formulations according to the present disclosure are stable at 5° C. and 25° C. over long storage times up to 18 months.

### 1.3.3. Turbidity Measurements

TABLE 7
Results of the turbidity at 860 nm in FNU over 18
months at different storage temperatures in syringes.

| | Tem- | F1 in | F2 in | F3 in | F4 in | F5 in |
|---|---|---|---|---|---|---|
| Time | perature | FNU | FNU | FNU | FNU | FNU |
| Initial | 25° C. | 6 | 6 | 5 | 8 | 5 |
| 3 months | | 6 | 6 | 6 | 8 | 5 |
| 6 months | | 6 | 5 | 4 | 7 | 5 |
| 8 months | | 7 | 7 | 6 | 9 | 7 |
| 12 months | | 8 | 7 | — | 9 | 6 |
| 18 months | | 9 | 7 | 7 | 10 | 7 |
| Initial | 5° C. | 6 | 6 | 5 | 8 | 5 |
| 6 months | | 6 | 5 | 4 | 8 | 4 |
| 8 months | | 6 | 5 | 4 | 7 | 4 |
| 18 months | | 7 | 6 | 5 | 9 | 5 |

The turbidity was measured at a wavelength of 860 nm, showing an increase in Formazine Nephelometric Units (FNU) of 1 to 3 during the storage time of 18 months at 25° C. Formulation 1 had the strongest increase in 3 FNU and formulation 2 the smallest increase in 1 FNU. Formulation 4 containing L-arginine had the highest turbidity from the beginning on.

The turbidity was measured at a wavelength of 860 nm, showing an increase of 0-1 FNU during the storage time of 18 months at 5° C.

### Results and Discussion

The turbidity measurement show that storage in a fridge over 18 months does not result in relative changes in turbidity. Formulation 4 containing L-arginine had the highest turbidity rendering arginine-free formulations more advantageous.

### 1.3.4. Conductivity Measurements

The conductivity of the formulations was measured.

TABLE 8
Results of the conductivity measurements in mS/cm at a
storage temperature at 25° C. for up to 18 months.

| | Tem- | | | | | |
|---|---|---|---|---|---|---|
| Time | perature | F1 | F2 | F3 | F4 | F5 |
| Initial | 25° C. | 1.25 | 1.37 | 1.53 | 3.96 | 1.54 |
| 3 months | | 1.08 | 1.27 | 1.40 | 3.71 | 1.41 |
| 6 months | | 1.29 | 1.34 | 1.52 | 3.85 | 1.52 |
| 8 months | | 1.14 | 1.22 | 1.35 | 3.66 | 1.42 |
| 12 months | | 1.18 | 1.23 | 1.43 | 3.61 | 1.43 |
| 18 months | | 1.15 | 1.26 | 1.39 | 4.05 | 1.38 |
| Initial | 5° C. | 1.25 | 1.37 | 1.53 | 3.96 | 1.54 |
| 6 months | | 1.25 | 1.35 | 1.48 | 3.92 | 1.54 |
| 8 months | | 1.12 | 1.18 | 1.36 | 3.67 | 1.38 |
| 18 months | | 1.07 | 1.14 | 1.29 | 3.99 | 1.36 |

### Results and Discussion

The conductivity remained constant for all 5 formulations over a storage time of 18 months and at a temperature of 5° C. and 25° C. While the conductivity of F1, F2, F3 and F5 has a value between 1 to 2 mS/cm, the L-arginine containing formulation F4 had a relatively high conductivity of just below 4 mS/cm.

### 1.4. Further Analytics and Results

In addition, further analytics were performed for the five formulations tested (storage times and temperature were as described above) with the following results.
The pH value remained essentially constant over the storage time of 18 months and at the different storage temperatures tested. Measured pH values were thus in a range from 5.7-6.3.
The osmolality remained essentially constant over the storage time of 18 months and at the different storage temperatures tested. The tested values ranged from 296-333 mOsm/kg.
The dynamic viscosity at 20° C. remained essentially constant over the storage time of 18 months and at the different storage temperatures tested. The dynamic viscosity was in a range of 10-14 mPas.
The protein concentration remained essentially constant over the storage time of 18 months and at the different storage temperatures tested. Small deviations of protein concentration are due to analytical variations, leading to ranges of 148-159 mg/mL.
The HP-SEC fragments contents remained essentially constant over the storage time of 18 months and at the different storage temperatures tested. The fragments content was in a range of 0.2-1.4%. The UP-SEC LMW contents remained essentially constant over the storage time of 18 months and at the different storage temperatures tested. In particular, a low increase of 3% was measured for 25° C. and 0.1-0.3% for 5° C. over 18 months. The LMW content was in a range of 1.0-4.5%.
The weak cation exchange (WCX) main peak, acidic peak group (APG) and basic peak group (BPG) content remained constant over the storage time of 18 months at 5° C. No differences between the formulations were observed regarding the main peak, APG and BPG.
The hydrophobic interaction chromatography (HIC) main peak content remained essentially constant over the storage time of 12 months at 25° C. or 8 months at 5° C.
The post peak increased by about 2-3% over at the storage temperature and time. The pre-peak was slightly increased by 5-7% over 12 months at 25° C. and did not increase at 5° C. over 8 months. No differences between the formulations were observed regarding the main peak, post peak and pre-peak.
The specific binding activity remained essentially constant over the storage time of 18 months and at the different storage temperatures tested with only minimal reduction of 3% and 1-2% for 25° C. and 5° C., respectively, for 18 months. The specific binding activity was in a range of 96-101%.
No visible particles were observed.

### 1.5. Summary of Results

All 5 formulations were stable over 18 months storage time at 25° C. and 5° C. Formulation 4, however, comprises an additional auxiliary agent. Formulation 3 was stable and did not have an additional auxiliary agent in contrast to the arginine-containing formulation F4. At a pH of about 5.7 less aggregate formation was observed.

2. Comparison of Acetate and Succinate Buffer Systems with Different pH Values in Free/Thaw Experiments

It was analyzed whether freezing and thawing of a 150 mg/mL formulation has an influence on the product quality of risankizumab. Therefore, 3 formulations were filled in mini bags having a volume of 10 mL or 14 mL for the initial value, followed by freezing at −40° C.

Furthermore, one bag was stored at 2-8° C. The storage time for both conditions (−40° C. and 2-8° C.) was 3 weeks,

The freezing was performed by a controlled freezing process. Afterwards, the bags were transferred into a −40° C. freezer and kept frozen for the indicated storage times.

### 2.1. Preparation of Formulations

Each freeze/thaw cycle comprised freezing at −40° C. in a lyophilizer and subsequent transfer into a −40° C. freezer and thawing in a lyophilizer at a maximal thawing rate of 20° C./min until room temperature after 3 weeks. The tested formulations are shown in Table 9.

TABLE 9
Composition of the formulations.

| | | Protein | | | |
|---|---|---|---|---|---|
| For- | | con- | | | |
| mula- | | centration | | | |
| tion | pH | mg/mL | buffer | Excipient 1 | Excipient 2 |
| F1 | 6.2 | 150 | 10 mM succinate | 185 mM | 0.02 w % PS20 |
| | | | | sorbitol | |
| F2 | 5.7 | 150 | 10 mM acetate | 185 mM | 0.02 w % PS20 |
| | | | 6.5 mM succinate | sorbitol | |
| F3 | 5.7 | 150 | 10 mM acetate | 160 mM | 0.02 w % PS20 |
| | | | 6.5 mM succinate | trehalose | |

The starting material was stored at 2-8° C. in a fridge until use. The bags (“Mini Flexboy Bags”) having a sample volume of 10 mL were frozen using a lyophilizer with a controlled freezing rate of 0.5° C./min until reaching a temperature of −40° C.

### 2.2. Analytics

The samples were directly before analysis thawed in a controlled manner using a lyophilizer. To measure the protein stability HP-SEC analysis was performed and the binding activity was measured. The turbidity was measured at 860 nm and at 400-600 nm. Further details on the utilized analysis methods are described below.

### 2.3. Results

### 2.3.1. Measurement of the Monomer and HMW Content

For determining the stability of the formulations, HP-SEC and UP-SEC analyses were performed, showing the monomer and HMW contents. Following results were obtained:

TABLE 10
Results HP-SEC monomer content in % after 3 weeks storage
(frozen at −40° C. and at 2-8° C.) and initial values.

| | F1-Bag | F1-Bag | F2-Bag | F2-Bag | F3-Bag | F3-Bag |
|---|---|---|---|---|---|---|
| | 1% | 2% | 1% | 2% | 1% | 2% |
| Initial | 98.7 | 98.6 | 98.9 | 98.9 | 98.9 | 98.9 |
| 3 weeks | 98.3 | 98.3 | 98.7 | 98.7 | 98.7 | 98.7 |
| 5° C. | | | | | | |
| 3 weeks | 98.6 | 98.6 | 98.9 | 98.9 | 98.9 | 98.9 |
| −40° C. | | | | | | |

The UP-SEC measurements confirm the results of the HP-SEC measurement.

TABLE 10a
Results UP-SEC monomer content in % after 3 weeks storage
(frozen at −40° C. and at 2-8° C.) and initial values.

| | F1-Bag | F1-Bag | F2-Bag | F2-Bag | F3-Bag | F3-Bag |
|---|---|---|---|---|---|---|
| | 1 % | 2% | 1% | 2% | 1% | 2% |
| Initial | 97.0 | 97.0 | 97.4 | 97.5 | 97.4 | 97.4 |
| 3 weeks | 97.0 | 97.0 | 97.4 | 97.4 | 97.3 | 97.3 |
| 5° C. | | | | | | |
| 3 weeks | 97.0 | 97.0 | 97.4 | 97.5 | 97.4 | 97.4 |
| −40° C. | | | | | | |

The remaining antibody content missing to 100% was present as HMW species.

### Results and Discussion

Overall, the results of the HP-SEC analysis demonstrate stability for all formulations, showing that the formulations allow for freeze/thaw cycle. Some formulations showed better results regarding a higher monomer content and less HMW species.

### 2.3.2. Measurement of the Binding Activity

The binding activity against rhIL-23 was measured utilizing surface plasmon resonance (Biacore) measurements. The binding activity is overall the same in all formulations, demonstrating applicability of the formulations. In particular, the binding activity was measured to be in a range between 95 to 110% and the specific binding activity was about 100%. The 3 weeks of storage in a frozen state or at 2-8° C. did not change the binding activity.

### 2.3.3. Measurement of the Viscosity

Another important parameter of protein formulations is the viscosity, which preferably is not too high in order to allow the formulation to be injected (e.g. pass a needle without excessive use of force). Therefore, the dynamic viscosity was measured.

The dynamic viscosity is very similar for F1, F2 and F3 being in a range of 8.7 to 10 mPas.

### 2.4. Further Analytics and Results

In addition, further analytics were performed for the three formulations tested with the following results.
The sub-visible particle content (≥25 μm, ≥10 μm, ≥5 μm) remained essentially constant over 3 weeks at 5° C. or at −40° C. including a freeze/thaw cycle. The sum of counted particles was essentially the same for all three formulations.
The osmolality remained essentially constant over 3 weeks at 5° C. or at −40° C. including a freeze/thaw cycle. The tested values ranged from 299-321 mOsm/kg for 150 mg/mL formulations.
The turbidity at 860 nm and 400-600 nm remained essentially constant over 3 weeks at 5° C. or at −40° C. including a freeze/thaw cycle. The tested values ranged from 2-7 FNU at 860 nm and 4-13 FNU at 400-600 nm.
The pH value remained essentially constant over 3 weeks at 5° C. or at −40° C. including a freeze/thaw cycle. Measured pH values were thus in a range from 5.7-6.2.
The conductivity remained essentially constant over 3 weeks at 5° C. or at −40° C. including a freeze/thaw cycle. Measured conductivity were thus in a range from 1.3-2.5 mS.
The protein concentration remained essentially constant over 3 weeks at 5° C. or at −40° C. including a freeze/thaw cycle. Small deviations of protein concentration are due to analytical variations, leading to ranges of 149-157 mg/mL for initial protein contents of 150 mg/mL.
The hydrophobic interaction chromatography (HIC) main peak content remained essentially constant over 3 weeks at 5° C. or at −40° C. including a freeze/thaw cycle.
The HIC main peak values ranged from 97.1-97.7%. The post peak and pre peak remained essentially constant over 3 weeks at 5° C. or at −40° C. including a freeze/thaw cycle.
The weak cation exchange (WCX) chromatography main peak content remained essentially constant over 3 weeks at 5° C. or at −40° C. including a freeze/thaw cycle.

The WCX main peak values range from 72.5-73.8%. The acidic peak group (APG) and basic peak group (BPG) remained essentially constant over 3 weeks at 5° C. or at −40° C. including a freeze/thaw cycle. No differences between the formulations were observed regarding main peak, APG and BGP.
The capillary gel electrophoresis (CGE) analysis showed essentially constant values over 3 weeks at 5° C. or at −40° C. including a freeze/thaw cycle. The non-reduced main peak contents ranged from 96.7-97.6%.

### 2.5. Summary of the Results

The results of the present example demonstrate stability of risankizumab provided in different 150 mg/mL formulations over freeze/thaw cycle. The single freeze/thaw cycle with a storage time of 3 weeks at −40° C. or at 2-8° C. for 3 weeks had no influence on the product quality of risankizumab. Therefore, 150 mg/mL formulations are a suitable concentration. A pH of 5.7 appeared to have performed slightly better compared to other pH values.

3. Comparison of Acetate and Succinate Buffer Systems with Different pH Values

A particularly suitable pH is in the range of 5.2 to 6.2 such as 5.5 to 6.2 or about 5.7. A higher pH can lead to increased protein aggregation, measured by SEC. A lower pH can lead to chemical degradation. In prior studies, sorbitol was used to adjust the tonicity. In the present example, trehalose and mannitol were used instead of sorbitol to adjust the tonicity. Seven sorbitol-free formulations were chosen and tested at following three conditions (i) 5° C. for 18 months, r.h. not monitored; (ii) 25° C./60% r.h. for 18 months and (iii) 40° C./75% r.h. for 6 months.

The compositions of formulation 1-7 are depicted below in Table 11.

TABLE 11
Composition of the analyzed formulations.

| Formulation | Composition |
|---|---|
| F1 | 150 mg/mL risankizumab, 10 mM |
| | acetate + 100 mM trehalose + 100 mM |
| | mannitol + 0.2 mg/mL PS20, pH 5.7 |
| F2 | 150 mg/mL risankizumab, 10 mM |
| | acetate + 200 mM trehalose + 0.2 |
| | mg/mL PS20, pH 5.7 |
| F3 | 150 mg/mL risankizumab, 10 mM |
| | acetate + 100 mM trehalose + 50 mM |
| | L-arginine HCl + 0.2 mg/mL PS20, pH 5.7 |
| F4 | 150 mg/mL risankizumab, 4.4 mM |
| | succinate + 200 mM trehalose + 0.2 |
| | mg/mL PS20, pH 6.0 |
| F5 | 150 mg/mL risankizumab, 4.4 mM |
| | succinate + 100 mM trehalose + 50 |
| | mM L-arginine HCl + 0.2 mg/mL |
| | PS20, pH 6.0 |
| F6 | 150 mg/mL risankizumab, 4.4 mM |
| | succinate + 100 mM trehalose + 100 |
| | mM mannitol + 0.2 mg/mL PS20, pH 6.0 |
| F7 | 150 mg/mL risankizumab, 200 mM |
| | trehalose + 0.2 mg/mL PS20, pH 5.7 |
| | (buffer free) |

The formulations and formulation buffers were sterile-filtered (filter type 0.22 m) and filled under laminar flow with a fill volume of 1.04 mL in syringes (Neopak). The formulations were prepared by mixing the starting material with a concentration solution comprising the auxiliary agents (excipients, buffer, etc.). The filled syringes were stored horizontally in rondotrays protected from light using cardboard boxes at 2-8° C. Following packing materials were used:
Neopak syringe (1 ml syringe with 27 gauge 12 inch needle)
Rubber stopper
Rondotray

### 3.1. Analytics

For analysis of the samples, HP-SEC and UP-SEC were performed and the turbidity (also referred to as opalescence) was measured. Following devices were used for analysis:
UPLC, UPSEC: UPLC 29/31 Waters ACQUITY, Waters, MA
HPLC, WCX/SEC: HPLC 82/83/107 Waters ALLIACE, Waters, MA
Particle count/size by MFI: Micro Flow Imagine, 5200 BOT A/B (Roboter), Protein Simple, GER
Osmometer: Osmomat 3000 Gonotec GmbH, GER
pH-meter: SevenGo, Mettler Toledo, GER
Turbidity photometer: 2100AN Turbidimeter, Hach-Lange GmbH, GER
Protein concentration by Solo VPE: Solo VPE, C. Technologies, Inc., NJ
Biacore: Biacore T200, GE Healthcare Life Science, UK
Tensile and Compression testing machine: Zwick 2.5 TS/N 21159574 Zwick, Germany

Further details on the utilized analysis methods are described below.

### 3.2. Results

### 3.2.1. Measurement of the Monomer Content

UP-SEC and HP-SEC were used to determine the loss of monomer content. Monomer content is a key quality attribute of protein stability and quality during stress-induced storage. The following table shows the results of the UP-SEC measurement.

TABLE 12
Monomer content measured by UP-SEC [%] of seven
formulations stored at 5° C., 25° C. and 40° C..

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 98.2 | 98.2 | 98.2 | 97.9 | 98.1 | 97.9 | 98.1 |
| | 4 | 97.8 | 97.8 | 98.0 | 97.5 | 97.8 | 97.5 | 97.8 |
| | 6 | 97.6 | 97.6 | 97.8 | 97.3 | 97.7 | 97.3 | 97.6 |
| | 9 | 97.5 | 97.5 | 97.7 | 97.0 | 97.5 | 97.0 | 97.4 |
| | 12 | 97.5 | 97.5 | 97.8 | 97.1 | 97.5 | 97.1 | 97.5 |
| | 18 | — | 97.3 | — | — | — | — | — |
| 25° C. | 0 | 98.2 | 98.2 | 98.2 | 97.9 | 98.1 | 97.9 | 98.1 |
| | 2 | 97.1 | 97.1 | 97.4 | 96.8 | 97.3 | 96.8 | 97.1 |
| | 4 | 96.5 | 96.5 | 96.9 | 96.3 | 96.8 | 96.2 | 96.5 |
| | 6 | 95.8 | 95.8 | 96.2 | 95.6 | 96.2 | 95.5 | 95.8 |
| | 9 | 95.1 | 95.1 | 95.5 | 94.9 | 95.5 | 94.9 | 95.2 |
| | 12 | 94.7 | 94.7 | 95.0 | 94.5 | 95.1 | 94.5 | 94.7 |
| | 18 | — | 93.3 | — | — | — | — | — |
| 40° C. | 0 | 98.2 | 98.2 | 98.2 | 97.9 | 98.1 | 97.9 | 98.1 |
| | 2 | 92.6 | 92.7 | 92.4 | 92.6 | 92.4 | 92.4 | 92.8 |
| | 4 | 88.4 | 88.5 | 87.7 | 88.6 | 88.2 | 88.5 | 88.8 |
| | 6 | 84.3 | 84.4 | 83.6 | 84.9 | 84.3 | 84.7 | 84.7 |

HP-SEC confirms the results of UP-SEC. Compared to UP-SEC, no additional information were generated by HP-SEC.

### Results and Discussion

All analyzed formulations were stable at the tested conditions.

### 3.2.2. Measurement of the HMW Level

UP-SEC and HP-SEC were used to determine levels of HMW formation. The following table shows the results of the UP-SEC measurement. The UMW content correlates with the monomer content. Loss of monomer leads to increase of HMVW.

TABLE 13
HMW measured by UP-SEC [%] of seven formulations
stored at 5° C., 25° C. and 40° C..

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 1 3 | 1.3 | 1.2 | 1.6 | 1.4 | 1.6 | 1.4 |
| | 4 | 1.5 | 1.6 | 1.4 | 1.9 | 1.5 | 1.9 | 1.6 |
| | 6 | 1.6 | 1.7 | 1.5 | 2.0 | 1.6 | 2.0 | 1.7 |
| | 9 | 1.8 | 1.8 | 1.6 | 2.3 | 1.8 | 2.3 | 1.9 |
| | 12 | 1.9 | 1.9 | 1.6 | 2.3 | 1.8 | 2.3 | 1.9 |
| | 18 | — | 2.0 | — | — | — | — | — |
| 25° C. | 0 | 0 | 1.3 | 1.3 | 1.2 | 1.6 | 1.4 | 1.6 |
| | 2 | 2.0 | 2.0 | 1.7 | 2.4 | 1.8 | 2.4 | 2.0 |
| | 4 | 2.2 | 2.2 | 1.8 | 2.5 | 1.9 | 2.5 | 2.2 |
| | 6 | 2.4 | 2.4 | 2.0 | 2.7 | 2.1 | 2.8 | 2.4 |
| | 9 | 2.7 | 2.7 | 2.2 | 3.0 | 2.3 | 3.0 | 2.7 |
| | 12 | 2.8 | 2.8 | 2.3 | 3.2 | 2.4 | 3.2 | 2.9 |
| | 18 | — | 3.2 | — | — | — | — | — |
| 40° C. | 0 | 0 | 1.3 | 1.3 | 1.2 | 1.6 | 1.4 | 1.6 |
| | 2 | 3.7 | 3.6 | 3.4 | 3.9 | 3.4 | 4.0 | 3.5 |
| | 4 | 5.2 | 5.1 | 5.1 | 5.3 | 4.8 | 5.4 | 4.9 |
| | 6 | 6.7 | 6.7 | 6.5 | 6.8 | 6.2 | 6.8 | 6.4 |

HP-SEC confirms the results of UP-SEC. Compared to UP-SEC, no additional information were generated by HP-SEC.

### Results and Discussion

Overall, all formulations were stable with low amounts of HMW species even after storage at 40° C.

### 3.2.3. Measurement of the LMW Level

UP-SEC was used to determine levels of LMW formation. The following table shows the results of the UP-SEC measurement.

TABLE 14
LMW content measured by UP-SEC [%] of seven
formulations stored at 5° C., 25° C. and 40° C..

| | storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 0.5 | 0.5 | 0.5 | 0.5 | 0.5 | 0.6 | 0.6 |
| | 4 | 0.6 | 0.6 | 0.7 | 0.6 | 0.7 | 0.7 | 0.7 |
| | 6 | 0.7 | 0.7 | 0.7 | 0.7 | 0.7 | 0.7 | 0.7 |
| | 9 | 0.7 | 0.7 | 0.7 | 0.7 | 0.7 | 0.7 | 0.7 |
| | 12 | 0.6 | 0.6 | 0.6 | 0.6 | 0.6 | 0.6 | 0.6 |
| | 18 | — | 0.7 | — | — | — | — | — |
| 25° C. | 0 | 0.5 | 0.5 | 0.5 | 0.5 | 0.5 | 0.6 | 0.6 |
| | 2 | 0.9 | 0.9 | 1.0 | 0.9 | 0.9 | 0.9 | 0.9 |
| | 4 | 1.3 | 1.3 | 1.4 | 1.3 | 1.3 | 1.3 | 1.3 |
| | 6 | 1.8 | 1.7 | 1.8 | 1.7 | 1.7 | 1.7 | 1.7 |
| | 9 | 2.2 | 2.2 | 2.4 | 2.1 | 2.2 | 2.1 | 2.2 |
| | 12 | 2.4 | 2.4 | 2.6 | 2.3 | 2.5 | 2.4 | 2.4 |
| | 18 | — | 3.6 | — | — | — | — | — |
| 40° C. | 0 | 0.5 | 0.5 | 0.5 | 0.5 | 0.5 | 0.6 | 0.6 |
| | 2 | 3.7 | 3.7 | 4.3 | 3.5 | 4.1 | 3.5 | 3.7 |
| | 4 | 6.4 | 6.4 | 7.2 | 6.1 | 7.0 | 6.1 | 6.4 |
| | 6 | 8.9 | 8.9 | 9.9 | 8.4 | 9.5 | 8.5 | 8.9 |

### Results and Discussion

Overall, all formulations were stable with low amounts of LMW species even after storage at 40° C.

### 3.2.4. Measurement of the LMW Level

For assessing the stability of the formulations, the LMW content was also measured by HP-SEC analysis. The results of this analysis are shown below.

TABLE 15
LMW content measured by HP-SEC [%] of seven formulations
stored at 5° C., 25° C. and 40° C..

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 0.2 | 0.2 | 0.2 | 0.2 | 0.2 | 0.2 | 0.2 |
| | 4 | 0.3 | 0.3 | 0.3 | 0.3 | 0.3 | 0.3 | 0.3 |
| | 6 | 0.3 | 0.3 | 0.3 | 0.3 | 0.3 | 0.3 | 0.3 |
| | 9 | 0.3 | 0.3 | 0.3 | 0.3 | 0.3 | 0.3 | 0.3 |
| | 12 | 0.4 | 0.3 | 0.3 | 0.3 | 0.3 | 0.3 | 0.3 |
| | 18 | — | 0.3 | — | — | — | — | — |
| 25° C. | 0 | 0.2 | 0.2 | 0.2 | 0.2 | 0.2 | 0.2 | 0.2 |
| | 2 | 0.4 | 0.4 | 0.4 | 0.4 | 0.4 | 0.4 | 0.4 |
| | 4 | 0.5 | 0.5 | 0.6 | 0.5 | 0.5 | 0.5 | 0.6 |
| | 6 | 0.6 | 0.6 | 0.6 | 0.6 | 0.6 | 0.6 | 0.6 |
| | 12 | 1.0 | 1.0 | 1.0 | 1.0 | 1.0 | 1.0 | 1.0 |
| | 18 | — | 1.2 | — | — | — | — | — |
| 40° C. | 0 | 0.2 | 0.2 | 0.2 | 0.2 | 0.2 | 0.2 | 0.2 |
| | 2 | 1.3 | 1.3 | 1.4 | 1.3 | 1.4 | 1.2 | 1.3 |
| | 4 | 2.2 | 2.2 | 2.4 | 2.1 | 2.3 | 2.1 | 2.2 |
| | 6 | 3.0 | 3.0 | 3.3 | 2.8 | 3.1 | 2.8 | 3.0 |

### Results and Discussion

The LMW content increases only slightly over time but all tested formulations generally resulted in low levels of fragmentation.

### 3.2.5. Turbidity Measurements

The results of opalescence are summarized below. No changes in opalescence were observed over the time of storage at the different conditions. The formulations containing L-arginine HCl (F3 and F5) showed the highest opalescence. However, there was no increasing opalescence in the course of the study for F3 and F5. In none of the tested formulations visible particles were observed.

TABLE 16
Opalescence [FNU] of seven formulations stored at
5° C., 25° C. and 40° C..

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 1 | 5 | 11 | 7 | 15 | 6 | 4 |
| | 4 | 5 | 5 | 11 | 7 | 13 | 7 | 4 |
| | 6 | 6 | 6 | 12 | 7 | 14 | 8 | 4 |
| | 9 | 5 | 5 | 11 | 7 | 15 | 8 | 6 |
| | 12 | 6 | 6 | 12 | 7 | 12 | 8 | 3 |
| | 18 | — | 6 | — | — | — | — | — |
| 25° C. | 0 | 5 | 5 | 11 | 7 | 15 | 6 | 4 |
| | 2 | 6 | 5 | 12 | 7 | 13 | 7 | 4 |
| | 4 | 5 | 5 | 13 | 7 | 13 | 7 | 3 |
| | 6 | 7 | 6 | 12 | 8 | 14 | 8 | 4 |
| | 9 | 8 | 7 | 13 | 9 | 14 | 8 | 4 |
| | 12 | 6 | 6 | 12 | 8 | 14 | 8 | 4 |
| | 18 | — | 7 | — | — | — | — | — |

### Results and Discussion

L-arginine HCL containing formulations showed increased opalescence.

### 3.3. Further Analytics and Results

In addition, further analytics were performed for the seven formulations tested with the following results (storage times and temperature were as described above).
The protein concentration remained essentially constant over the storage time of 18 months and at the different storage temperatures tested.
The pH value remained essentially constant over the storage time of 18 months and at the different storage temperatures tested.
The osmolality remained essentially constant over the storage time of 18 months and at the different storage temperatures tested. The tested values ranged from 298-326 mOsm/kg.
No visible particles were observed.
The IEC/WCX measurements of formulations at 5° C. for the tested storage times showed constant levels of main peak (69.7-72.2%), APG (18-20%) and BPG (8-13%). At 25° C. and 40° C. the main peak decreases and APG level increases for all formulations in a similar range. At 25° C. and 40° C. BPG level of formulations with pH 6.0 is lightly lower (up to 2%) than formulations with pH 5.7.
The particle content measured by MFI remained essentially constant over the storage time of 18 months and at the different storage temperatures tested. In particular, for particles ≥10 μm and >25 μm no relevant increasing particle count could be observed for all formulations over time of storage at 5° C. and 25° C./60% r.h. For particles ≥2 μm, the particle count at 25° C. was increased. The increase was for all formulations in a similar range.
The specific binding activity remained essentially constant over the storage time of 18 months and at the different storage temperatures tested. The specific binding activity was in a range of 95-100%.
The break loose and gliding forces remained essentially constant over the storage time of 18 months and at 5° C. At 25° C. the maximum gliding force, average gliding force and break loose force increased over time of storage for all formulations. No differences between the formulations were observed. Maximum gliding forces ranged from 7.1-23.6 N, average gliding forces from 6.7-20.5 N and break loose force from 3.4-7.1 N.
The dynamic viscosity measured at 20° C. remained essentially constant over the storage time of 18 months and at the different storage temperatures tested. The dynamic viscosity was in a range of 8.3-10.7 mPas.

### 3.4. Summary of Results

The present example describes the storage stability of seven different 150 mg/mL risankizumab formulations. Trehalose and mannitol instead of sorbitol were used to adjust the tonicity. F2 was additionally analyzed after 18 months.

UP-SEC showed that a pH of 6.0 compared to pH 5.7 leads in the tested formulations to a slightly larger degradation of risankizumab in form of lower monomer content and higher HMW. L-arginine HCl containing formulations showed slightly lower degradation measured by UP-SEC but increased opalescence and slightly increased LMW contents. IEC showed no relevant differences between the formulations and were therefore not a decisive factor. The same applies to pH, protein concentration, osmolality, viscosity, break loose and gliding force, subvisible particles and visible particles. No differences between the formulations were seen by this data.

4. Comparison of Acetate and Succinate Buffer Systems with Different pH Values Over Long Term Storage

The present example analyzes the storage stability of seven different 150 mg/mL risankizumab formulations in Neopak syringes to analyze the storage stability of the formulations and identify advantageous formulations. In comparison to the seven formulations of the prior example, the tonicity was slightly modified. Three conditions were again tested (as identified above). The formulations analyzed are summarized in Table 17.

TABLE 17
Composition of the analyzed formulations.

| Formulation | Composition |
|---|---|
| F1 | 150 mg/mL risankizumab, 10 mM |
| | acetate + 95 mM trehalose + 95 mM |
| | mannitol + 0.2 mg/mL PS20, pH 5.7 |
| F2 | 150 mg/mL risankizumab, 10 mM |
| | acetate + 185 mM trehalose + 0.2 |
| | mg/mL PS20, pH 5.7 |
| F3 | 150 mg/mL risankizumab, 10 mM |
| | acetate + 110 mM trehalose + 50 mM |
| | L-arginine HCl + 0.2 mg/mL PS20, pH 5.7 |
| F4 | 150 mg/mL risankizumab, 4.4 mM |
| | succinate + 180 mM trehalose + 0.2 |
| | mg/mL PS20, pH 6.0 |
| F5 | 150 mg/mL risankizumab, 4.4 mM |
| | succinate + 110 mM trehalose + 50 |
| | mM L-arginine HCl + 0.2 mg/mL PS20, pH 6.0 |
| F6 | 150 mg/mL risankizumab, 4.4 mM |
| | succinate + 95 mM trehalose + 95 mM |
| | mannitol + 0.2 mg/mL PS20, pH 6.0 |
| F7 | 150 mg/mL risankizumab, 200 mM |
| | trehalose + 0.2 mg/mL PS20, pH 5.7 |
| | (buffer free) |

The formulations were prepared by mixing the starting material with a concentrated spike solution (comprising the auxiliary agents, i.e. the excipients and buffers).

### 4.1. Analytics

For analysis of the samples, inter alia UP-SEC was performed and the opalescence was measured. Further details on the utilized analysis methods are described below.

### 4.2. Results

### 4.2.1. Measurement of the HMW Content

TABLE 18
Measured HMW content in % of the UP-SEC analysis for
long-term storage at 5° C., 25° C. and 40° C..

| | Storage | | | | | | | |
|---|---|---|---|---|---|---|---|---|
| Storage | time, | F1 | F2 | F3 | F4 | F5 | F6 | F7 |
| condition | months | in % | in % | in % | in % | in % | in % | in% |
| 5° C. | 0 | 1.3 | 1.3 | 1.2 | 1.6 | 1.4 | 1.6 | 1.4 |
| | 4 | 1.5 | 1.6 | 1.4 | 1.9 | 1.5 | 1.9 | 1.6 |
| | 6 | 1.6 | 1.7 | 1.5 | 2.0 | 1.6 | 2.0 | 1.7 |
| | 9 | 1.8 | 1.8 | 1.6 | 2.3 | 1.8 | 2.3 | 1.9 |
| | 12 | 1.9 | 1.9 | 1.6 | 2.3 | 1.8 | 2.3 | 1.9 |
| | 18 | — | 2.0 | — | — | — | — | — |
| 25° C. | 0 | 0 | 1.3 | 1.3 | 1.2 | 1.6 | 1.4 | 1.6 |
| | 2 | 2.0 | 2.0 | 1.7 | 2.4 | 1.8 | 2.4 | 2.0 |
| | 4 | 2.2 | 2.2 | 1.8 | 2.5 | 1.9 | 2.5 | 2.2 |
| | 6 | 2.4 | 2.4 | 2.0 | 2.7 | 2.1 | 2.8 | 2.4 |
| | 9 | 2.7 | 2.7 | 2.2 | 3.0 | 2.3 | 3.0 | 2.7 |
| | 12 | 2.8 | 2.8 | 2.3 | 3.2 | 2.4 | 3.2 | 2.9 |
| | 18 | — | 3.2 | — | — | — | — | — |
| 40° C. | 0 | 0 | 1.3 | 1.3 | 1.2 | 1.6 | 1.4 | 1.6 |
| | 2 | 3.7 | 3.6 | 3.4 | 3.9 | 3.4 | 4.0 | 3.5 |
| | 4 | 5.2 | 5.1 | 5.1 | 5.3 | 4.8 | 5.4 | 4.9 |
| | 6 | 6.7 | 6.7 | 6.5 | 6.8 | 6.2 | 6.8 | 6.4 |

### Results and Discussion

The HMW content remained overall low in all tested formulations indicating that the used formulations can stabilize risankizumab at high concentrations. The UP-SEC results further show that a pH of 6.0 compared to pH 5.7 leads to a larger degradation of risankizumab in form of lower monomer contents and higher HMW levels. Therefore, a pH of 5.7 is particularly advantageous for formulations according to the present disclosure. Nevertheless, also the formulations having a higher pH value of 6.0 such as F4, F5 and F6 showed overall good performance in view of the UP-SEC analysis results.

### 4.2.2. Measurement of the LMW Content

TABLE 19
Measured LMW content in % of the UP-SEC analysis
for long-term storage at 5° C., 25° C. and 40° C.

| | Storage | F1 | F2 | F3 | F4 | F5 | F6 | F7 |
|---|---|---|---|---|---|---|---|---|
| Storage | time, | in | in | in | in | in | in | in |
| condition | months | % | % | % | % | % | % | % |
| 5° C. | 0 | 0.5 | 0.5 | 0.5 | 0.5 | 0.5 | 0.6 | 0.6 |
| | 4 | 0.6 | 0.6 | 0.7 | 0.6 | 0.7 | 0.7 | 0.7 |
| | 6 | 0.7 | 0.7 | 0.7 | 0.7 | 0.7 | 0.7 | 0.7 |
| | 9 | 0.7 | 0.7 | 0.7 | 0.7 | 0.7 | 0.7 | 0.7 |
| | 12 | 0.6 | 0.6 | 0.6 | 0.6 | 0.6 | 0.6 | 0.6 |
| | 18 | — | 0.7 | — | — | — | — | — |
| 25° C. | 0 | 0.5 | 0.5 | 0.5 | 0.5 | 0.5 | 0.6 | 0.6 |
| | 2 | 0.9 | 0.9 | 1.0 | 0.9 | 0.9 | 0.9 | 0.9 |
| | 4 | 1.3 | 1.3 | 1.4 | 1.3 | 1.3 | 1.3 | 1.3 |
| | 6 | 1.8 | 1.7 | 1.8 | 1.7 | 1.7 | 1.7 | 1.7 |
| | 9 | 2.2 | 2.2 | 2.4 | 2.1 | 2.2 | 2.1 | 2.2 |
| | 12 | 2.4 | 2.4 | 2.6 | 2.3 | 2.5 | 2.4 | 2.4 |
| | 18 | — | 3.6 | — | — | — | — | — |
| 40° C. | 0 | 0.5 | 0.5 | 0.5 | 0.5 | 0.5 | 0.6 | 0.6 |
| | 2 | 3.7 | 3.7 | 4.3 | 3.5 | 4.1 | 3.5 | 3.7 |
| | 4 | 6.4 | 6.4 | 7.2 | 6.1 | 7.0 | 6.1 | 6.4 |
| | 6 | 8.9 | 8.9 | 9.9 | 8.4 | 9.5 | 8.5 | 8.9 |

### Results and Discussion

The tested formulations were stable over the measurement time for all tested temperatures. Therefore, high protein concentrations of 150 mg/mL risankizumab were effectively stabilized using the tested formulations. At higher storage temperature, a slight increase of LMW content was observed for L-arginine HCl containing formulations. This is a surprising finding, as L-arginine containing formulations typically are known to further stabilize formulations. Hence, a formulation of 150 mg/mL risankizumab is different from other protein formulations in this respect. A formulation according to the present disclosure without arginine is therefore preferred.

### 4.2.3. Measurement of the Opalescence

TABLE 20
Measured opalescence in FNU for long-term
storage at 5° C., 25° C. and 40° C.

| | Storage | F1 | F2 | F3 | F4 | F5 | F6 | F7 |
|---|---|---|---|---|---|---|---|---|
| Storage | time, | in | in | in | in | in | in | in |
| condition | months | FNU | FNU | FNU | FNU | FNU | FNU | FNU |
| 5° C. | 0 | 1 | 5 | 11 | 7 | 15 | 6 | 4 |
| | 4 | 5 | 5 | 11 | 7 | 13 | 7 | 4 |
| | 6 | 6 | 6 | 12 | 7 | 14 | 8 | 4 |
| | 9 | 5 | 5 | 11 | 7 | 15 | 8 | 6 |
| | 12 | 6 | 6 | 12 | 7 | 12 | 8 | 3 |
| | 18 | — | 6 | — | — | — | — | — |
| 25° C. | 0 | 5 | 5 | 11 | 7 | 15 | 6 | 4 |
| | 2 | 6 | 5 | 12 | 7 | 13 | 7 | 4 |
| | 4 | 5 | 5 | 13 | 7 | 13 | 7 | 3 |
| | 6 | 7 | 6 | 12 | 8 | 14 | 8 | 4 |
| | 9 | 8 | 7 | 13 | 9 | 14 | 8 | 4 |
| | 12 | 6 | 6 | 12 | 8 | 14 | 8 | 4 |
| | 18 | — | 7 | — | — | — | — | — |

### Results and Discussion

Overall, no or only a slight increase in opalescence was observed over time, indicating stability of all tested formulations. For formulations which contain L-arginine HCl (F3 and F5) a higher opalescence was observed.

### 4.3. Summary of Results

The measured parameters show that all formulations are suitable for preparing high risankizumab concentrations of 150 mg/mL in a stable manner. The long-term stability revealed some differences between the formulations:
UP-SEC showed that a pH of 6.0 compared to pH 5.7 leads to a larger degradation of risankizumab in form of lower monomer content and higher HMW.
L-arginine HCl containing formulations showed lower degradation measured by UP-SEC but increased opalescence and slight increase of LMW.

F2 was additionally analyzed after 18 months.

Noteworthy, also formulations F1 and F7 were stable. Buffer-free formulations and formulations comprising more than one type of tonicity agent were also stable and are thus suitable to provide a formulation comprising 150 mg/mL risankizumab.

In summary, formulation F2 was found to be particularly stable in respect to the measured LMW and HMW content, as well as opalescence, indicating a superior stability. This result was very surprising, as typically risankizumab is known to be used at higher pH values. Hence, the particularly high concentration of risankizumab shifts the optimal pH value to about 5.7, which was unexpected. Moreover, it was surprising that L-arginine HCl did not result in further stabilization but in fact reduced the stability of the formulations, supported by the higher opalescence and measured LMW content. Consequently, the particular properties of risankizumab at a high concentration, e.g. 150 mg/mL, necessitate different optimal conditions than formerly known formulations of risankizumab.

5. Comparison of Acetate and Succinate Buffer Systems with Different pH Values while Shaking

The goal of the present example was to evaluate the influence of shaking stress on the product quality of different formulations at 150 mg/mL risankizumab. Different formulations were tested regarding their ability to stabilize risankizumab against shaking stress. Therefore, the formulation was exposed to different shaking stresses at the antibody concentration of 150 mg/mL.

In total eleven formulations that differ in pH, buffering and tonicity agent were filled into 6R vials and 1 mL Neopak syringes with 27 gauge ½ inch needles and were shaken for 21 days at room temperature. Corresponding buffer solutions without protein were shaken, stored and analyzed as well. Shaking conditions:
Shaking temperature: room temperature (approx. 25° C.)
Shaking time: 21 days
Type of shaking: Horizontal shaker (vial), rocking shaker (syringe); the shaking was conducted protected from light

To exclude the influence of temperature as an additional stress on product quality further vials and syringes were stored at room temperature without shaking.

### 5.1. Preparation of Formulations

11 risankizumab test formulations were prepared (see table 21) and subjected to:
a) shaking of vials for 1, 5, 7, 14, 21 days in a horizontal shaker with 300 U/min (protected from light);
b) shaking of syringes for 1, 5, 7, 14, 21 days in a rocking shaker, movement adjusted to respective viscosity to ensure air bubble movement (protected from light); and
c) room temperature (25° C.) for 1, 5, 7, 14, 21 days (protected from light).

TABLE 21
Formulations chosen for shaking study.

| | |
|---|---|
| F1 | 10 mM acetate + 6.5 mM succinate + |
| | 185 mM sorbitol + 0.02 % PS20, pH |
| | 5.7 |
| F2 | 10 mM acetate + 200 mM sorbitol + |
| | 0.02 % PS20, pH 5.7 |
| F3 | 10 mM acetate + 50 mM L-arginine |
| | HCl + 110 mM sorbitol + 0.02 % |
| | PS20, pH 5.7 |
| F4 | 10 mM succinate + 185 mM |
| | sorbitol + 0.02 % PS20, pH 6.2 |
| F5 | 10 mM acetate + 95 mM mannitol + 95 |
| | mM trehalose + 0.02 % PS20, pH |
| | 5.7 |
| F6 | 10 mM acetate + 185 mM trehalose + |
| | 0.02 % PS20, pH 5.7 |
| F7 | 10 mM acetate + 50 mM L-arginine |
| | HCl + 110 mM trehalose + 0.02% |
| | PS20, pH 5.7 |
| F8 | 4.4 mM succinate + 185 mM trehalose + |
| | 0.02 % PS20, pH 6.0 |
| F9 | 4.4 mM succinate + 50 mM L-arginine |
| | HCl + 110 mM trehalose + 0.02% |
| | PS20, pH 6.0 |
| F10 | 4.4 mM succinate + 95 mM mannitol + |
| | 95 mM trehalose + 0.02 % PS20, |
| | pH 6.0 |
| F11 | Buffer-free, 200 mM trehalose, 0.02 % PS20, pH 5.7 |

As a packaging material the formulations were added to vials (Schott) or Neopak syringes. The sterile filtered protein solutions were filled under laminar flow into the sterilized primary packaging materials. The filling volume for the vials was defined to be 3.6 mL. The syringes were filled with 1.04 mL each. All vials and syringes were inspected for visual particles and results were recorded.

### 5.2. Analytics

The analytics at each analytical time point were performed directly after sampling except for the chromatographic assays like SEC for which samples were stored at −70° C. until measurement. Following devices were used for analysis:
UV-Vis spectrophotometer Solo VPE: Conc. at 280 nm, baseline correction at 320 nm, extinction coefficient: 1.52; C Technologies, Inc., NJ, USA
Opalescence meter: HACH Lange opalescence meter; filter: 400-600 nm; Hach Lange GmbH, Dusseldorf, Germany
Ultra performance size exclusion chromatography (UP-SEC): UPLC26, H-Class UV-detection at 280 nm Waters, Milford, MA
Charge heterogeneity by weak cation exchange chromatography (WCX): HPLC75; Fluorescence detection Extinction: 278 nm, Emission: 350 nm; Waters, Milford, MA
IL-23 binding activity: Biacore T200 Chip: CM5 GE Healthcare, Chalfont St Giles, UK
pH-meter: SevenGo—Mettler Toledo, Columbus, OH
Particel sizer: Micro Flow Imaging™ Flow Microscope; By micro flow imaging (MFI); Brightwell Technologies Inc, Ottawa, ON, Canada
Osmometer: Osmomat 030 By freezing point depression, Gonotec GmbH, Berlin, Germany

Further details on the utilized analysis methods are described below.

### 5.3. Results

### 5.3.1. Measurement of the Monomer Content

Monomer content is a key quality attribute of protein stability and quality during stress-induced storage. HP-SEC and UP-SEC were used to measure the monomer content of the formulations.

### UP-SEC Analysis

TABLE 22
UP-SEC-Monomer in % of syringe and vial: Initial values and values after 1 / 5 / 7 / 14 / 21 days
of shaking and after 21 days without movement at 25° C.

| | | F1 | F2 | F3 | F4 | F5 | F6 | F7 | F8 | F9 | F10 | F11 |
|---|---|---|---|---|---|---|---|---|---|---|---|---|
| Time | Packing | (%) | (%) | (%) | (%) | (%) | (%) | (%) | (%) | (%) | (%) | (%) |
| Initial | Syringe | 97.9 | 97.9 | 97.9 | 97.5 | 97.8 | 97.8 | 97.9 | 97.4 | 97.5 | 97.5 | 97.7 |
| 1 day | | 97.6 | 97.6 | 97.7 | 97.0 | 97.6 | 97.6 | 97.7 | 97.3 | 97.5 | 97.3 | 97.6 |
| 5 days | | 97.5 | 97.5 | 97.6 | 96.9 | 97.5 | 97.5 | 97.6 | 97.2 | 97.4 | 97.2 | 97.5 |
| 7 days | | 97.4 | 97.4 | 97.5 | 96.7 | 97.4 | 97.3 | 97.5 | 97.0 | 97.3 | 96.9 | 97.3 |
| 14 days | | 97.3 | 97.2 | 97.4 | 96.6 | 97.2 | 97.2 | 97.4 | 96.9 | 97.2 | 96.8 | 97.2 |
| 21 days | | 97.3 | 97.3 | 97.4 | 96.7 | 97.2 | 97.3 | 97.4 | 96.9 | 97.2 | 96.9 | 97.3 |
| 21 days* | | 97.3 | 97.3 | 97.5 | 96.8 | 97.3 | 97.3 | 97.5 | 97.1 | 97.4 | 97.0 | 97.4 |
| Initial | Vial | 97.9 | 97.8 | 97.9 | 97.5 | 97.8 | 97.7 | 97.9 | 97.6 | 97.8 | 97.7 | 97.9 |
| 1 day | | 97.7 | 97.7 | 97.8 | 97.3 | 97.7 | 97.7 | 97.8 | 97.5 | 97.7 | 97.5 | 97.7 |
| 5 days | | 97.5 | 97.5 | 97.6 | 97.0 | 97.5 | 97.5 | 97.6 | 97.3 | 97.5 | 97.3 | 97.6 |
| 7 days | | 97.5 | 97.5 | 97.6 | 97.0 | 97.5 | 97.5 | 97.6 | 97.2 | 97.5 | 97.3 | 97.4 |
| 14 days | | 97.3 | 97.3 | 97.4 | 96.7 | 97.2 | 97.2 | 97.4 | 97.0 | 97.3 | 97.0 | 97.3 |
| 21 days | | 97.2 | 97.2 | 97.4 | 96.7 | 97.2 | 97.2 | 97.4 | 96.9 | 97.2 | 97.0 | 97.3 |
| 21 days* | | 97.3 | 97.3 | 97.4 | 96.7 | 97.2 | 97.2 | 97.4 | 97.0 | 97.3 | 97.0 | 97.3 |

*corresponds to no movement, whereas the other samples were shaken for the indicated amount of time.

### HP-SEC Analysis

The trends of the HP-SEC analysis are analogous to those of the UP-SEC and thus confirm these results. Monomer values ranging from 97.4-98.8% were obtained.

### Results and Discussion

Overall, the measurements in syringes and vials show similar trends and all formulations proved to be stable with only slight reductions in monomer content.

### 5.3.2. Measurement of the HMW Content

HP-SEC and UP-SEC were used to measure the monomer content of the formulations. HP-SEC was used to determine levels of aggregate (HMW) formation during shaking of the syringes and vials.

### UP-SEC Analysis

The results of the UP-SEC analysis are shown below. The data show similar results as the HP-SEC analysis that the formation of aggregates is mainly driven by the pH. When comparing the initial values it is obvious that formulations at pH 6.0 or 6.2 show a slightly increased HMW content from 0.2 to 0.5% in comparison to solutions formulated at pH 5.7. This trend could also be seen after 21 days of shaking with HMW contents of approx. 1.6% in formulations ≥pH 6.0 and 1.3% in formulations at pH 5.7.

Formulations containing L-arginine like F3 and F7 showed the lowest level of aggregation after 21 days of shaking. The differences in monomer content for the eleven formulations tested in this study observed in UP-SEC and HP-SEC are not significant. The loss of monomer content was in an acceptable range for all formulations being tested. Generally, it can be summarized that the shaking does not significantly increase the HMW content in comparison to the results after 21 days without movement.

The data obtained using the UP-SEC is summarized in Table 23.

TABLE 23
UP-SEC HMW content in % of syringe and vial: Initial values and values after 1 / 5 / 7 / 14 / 21
days of shaking and after 21 days without movement at 25° C.

| | | F1 | F2 | F3 | F4 | F5 | F6 | F7 | F8 | F9 | F10 | F11 |
|---|---|---|---|---|---|---|---|---|---|---|---|---|
| Time | Packing | (%) | (%) | (%) | (%) | (%) | (%) | (%) | (%) | (%) | (%) | (%) |
| Initial | Syringe | 0.9 | 0.9 | 0.8 | 1.3 | 0.9 | 0.9 | 0.8 | 1.1 | 1.0 | 1.0 | 0.9 |
| 1 day | | 1.0 | 1.0 | 1.0 | 1.6 | 1.0 | 1.0 | 0.9 | 1.3 | 1.1 | 1.3 | 1.0 |
| 5 days | | 1.1 | 1.1 | 1.1 | 1.8 | 1.2 | 1.2 | 1.0 | 1.4 | 1.2 | 1.4 | 1.1 |
| 7 days | | 1.2 | 1.3 | 1.1 | 2.0 | 1.3 | 1.3 | 1.2 | 1.7 | 1.4 | 1.7 | 1.3 |
| 14 days | | 1.3 | 1.4 | 1.2 | 2.0 | 1.4 | 1.4 | 1.2 | 1.7 | 1.4 | 1.8 | 1.4 |
| 21 days | | 1.4 | 1.4 | 1.3 | 2.0 | 1.5 | 1.5 | 1.2 | 1.7 | 1.5 | 1.8 | 1.4 |
| 21 days* | | 1.4 | 1.3 | 1.2 | 1.9 | 1.4 | 1.4 | 1.2 | 1.6 | 1.3 | 1.6 | 1.3 |
| Initial | Vial | 0.9 | 0.9 | 0.8 | 1.3 | 0.9 | 0.9 | 0.8 | 1.1 | 0.9 | 1.1 | 0.9 |
| 1 day | | 1.0 | 1.0 | 0.9 | 1.4 | 1.0 | 1.0 | 0.9 | 1.2 | 1.0 | 1.2 | 1.0 |
| 5 days | | 1.1 | 1.1 | 1.0 | 1.6 | 1.1 | 1.1 | 1.0 | 1.4 | 1.2 | 1.3 | 1.1 |
| 7 days | | 1.2 | 1.2 | 1.1 | 1.7 | 1.2 | 1.2 | 1.0 | 1.4 | 1.1 | 1.4 | 1.2 |
| 14 days | | 1.4 | 1.4 | 1.2 | 1.9 | 1.4 | 1.4 | 1.2 | 1.7 | 1.3 | 1.7 | 1.3 |
| 21 days | | 1.4 | 1.4 | 1.3 | 1.9 | 1.4 | 1.4 | 1.2 | 1.7 | 1.4 | 1.7 | 1.4 |
| 21 days* | | 1.4 | 1.4 | 1.3 | 2.0 | 1.5 | 1.5 | 1.2 | 1.7 | 1.4 | 1.7 | 1.4 |

*corresponds to no movement whereas the other samples were shaken for the indicated amount of time.

### HP-SEC Analysis

The trend of the data of the UP-SEC analysis are analogous to those of the UP-SEC and thus confirm these results.

### Results and Discussion

Overall, all tested formulations proved to be stable. Similar results were obtained for vials and syringes.

### 5.3.3. Measurement of the Opalescence and Further Parameters

Opalescence, osmolality, pH-values and protein concentrations of all tested formulations remained essentially unchanged following 21 days of shaking for syringes on a horizontal shaker as well as for vials on a rocking shaker (see subsequent data). The lowest level of opalescence was observed in the buffer-free formulation F11 without any additional buffer like acetate or succinate. No observations could be made with regard to visual inspection. No significant difference can be observed by comparing the generated data of vials and syringes.

The increased opalescence level of F6 after 1 day and F10 after 5 days could not be confirmed by the results of the following sampling time points. Therefore, it is likely that a measuring error occurred and these results do not have relevance for the interpretation of the results.

TABLE 24
Shaking of formulations in syringe and vial: Initial values, values after 1 / 5 / 7 / 14 / 21
days of shaking and values after 21 days without (w/o) movement of opalescence,
osmolality, pH-values and protein conc. (shaking at room temperature).

| | | Opalescence (FNU) | | Osmolality (mOsm/kg) | | pH | | Protein (mg/mL) | |
|---|---|---|---|---|---|---|---|---|---|
| Formulation | Time | Syringe | Vial | Syringe | Vial | Syringe | Vial | Syringe | Vial |
| F1 | Initial | 9.0 | 8.1 | 307 | 306 | 5.6 | 5.7 | 150 | 152 |
| | 1 day | 8.1 | 8.3 | 304 | 305 | 5.7 | 5.7 | 151 | 151 |
| | 5 days | 7.6 | 9.2 | 307 | 305 | 5.7 | 5.8 | 151 | 151 |
| | 7 days | 8.5 | 9.0 | 307 | 306 | 5.7 | 5.7 | 148 | 150 |
| | 14 days | 8.6 | 8.3 | 305 | 307 | 5.7 | 5.7 | 152 | 151 |
| | 21 days | 8.8 | 8.6 | 304 | 306 | 5.8 | 5.7 | 150 | 149 |
| | 21 days* | 9.6 | 8.0 | 308 | 304 | 5.7 | 5.7 | 151 | 149 |
| F2 | Initial | 5.6 | 5.4 | 306 | 307 | 5.6 | 5.6 | 151 | 152 |
| | 1 day | 5.9 | 5.7 | 304 | 305 | 5.7 | 5.7 | 149 | 152 |
| | 5 days | 5.2 | 5.6 | 305 | 305 | 5.7 | 5.7 | 149 | 149 |
| | 7 days | 5.7 | 5.8 | 304 | 305 | 5.7 | 5.7 | 149 | 149 |
| | 14 days | 5.4 | 5.4 | 306 | 307 | 5.7 | 5.7 | 151 | 151 |
| | 21 days | 6.5 | 7.0 | 304 | 304 | 5.7 | 5.7 | 151 | 150 |
| | 21 days* | 5.7 | 4.8 | 309 | 305 | 5.7 | 5.7 | 150 | 151 |
| F3 | Initial | 12.8 | 11.3 | 298 | 289 | 5.6 | 5.7 | 154 | 152 |
| | 1 day | 13.0 | 12.2 | 289 | 282 | 5.7 | 5.8 | 151 | 149 |
| | 5 days | 12.7 | 12.7 | 291 | 286 | 5.6 | 5.7 | 153 | 151 |
| | 7 days | 12.2 | 13.3 | 292 | 291 | 5.7 | 5.7 | 153 | 149 |
| | 14 days | 12.2 | 11.9 | 293 | 291 | 5.7 | 5.7 | 152 | 151 |
| | 21 days | 12.7 | 12.2 | 291 | 290 | 5.7 | 5.7 | 153 | 151 |
| | 21 days* | 13.0 | 11.3 | 293 | 292 | 5.7 | 5.7 | 152 | 154 |
| F4 | Initial | 13.4 | 11.1 | 301 | 294 | 6.1 | 6.2 | 151 | 150 |
| | 1 day | 11.4 | 11.4 | 296 | 297 | 6.0 | 6.3 | 151 | 152 |
| | 5 days | 11.9 | 12.4 | 297 | 296 | 6.0 | 6.2 | 150 | 148 |
| | 7 days | 11.7 | 12.8 | 299 | 295 | 6.2 | 6.2 | 153 | 149 |
| | 14 days | 11.9 | 11.2 | 298 | 296 | 6.2 | 6.2 | 152 | 153 |
| | 21 days | 12.2 | 12.3 | 301 | 298 | 6.2 | 6.2 | 151 | 151 |
| | 21 days* | 12.9 | 11.5 | 297 | 299 | 6.2 | 6.2 | 153 | 152 |
| F5 | Initial | 5.9 | 4.9 | 310 | 306 | 5.6 | 5.7 | 156 | 154 |
| | 1 day | 5.6 | 5.3 | 306 | 303 | 5.7 | 5.7 | 154 | 153 |
| | 5 days | 5.7 | 5.8 | 305 | 305 | 5.6 | 5.7 | 154 | 154 |
| | 7 days | 5.5 | 6.0 | 309 | 304 | 5.7 | 5.7 | 153 | 151 |
| | 14 days | 4.9 | 5.0 | 306 | 305 | 5.7 | 5.7 | 156 | 157 |
| | 21 days | 5.4 | 7.5 | 306 | 308 | 5.7 | 5.7 | 155 | 154 |
| | 21 days* | 6.3 | 4.7 | 307 | 305 | 5.6 | 5.7 | 154 | 153 |
| F6 | Initial | 5.7 | 4.9 | 313 | 311 | 5.7 | 5.7 | 153 | 151 |
| | 1 day | 11.0 | 6.1 | 304 | 307 | 5.6 | 5.8 | 152 | 151 |
| | 5 days | 6.0 | 6.1 | 308 | 305 | 5.6 | 5.7 | 150 | 149 |
| | 7 days | 5.6 | 5.9 | 307 | 308 | 5.7 | 5.7 | 151 | 151 |
| | 14 days | 5.2 | 4.9 | 307 | 309 | 5.7 | 5.7 | 150 | 151 |
| | 21 days | 5.7 | 6.5 | 311 | 310 | 5.7 | 5.7 | 152 | 152 |
| | 21 days* | 5.5 | 4.8 | 311 | 310 | 5.7 | 5.7 | 153 | 150 |
| F7 | Initial | 12.3 | 10.8 | 307 | 304 | 5.6 | 5.7 | 153 | 153 |
| | 1 day | 12.3 | 11.3 | 302 | 302 | 5.6 | 5.7 | 153 | 154 |
| | 5 days | 12.1 | 12.5 | 301 | 302 | 5.6 | 5.7 | 151 | 153 |
| | 7 days | 11.4 | 12.6 | 302 | 302 | 5.7 | 5.7 | 153 | 154 |
| | 14 days | 12.2 | 10.9 | 304 | 303 | 5.7 | 5.7 | 153 | 152 |
| | 21 days | 11.5 | 12.1 | 306 | 304 | 5.7 | 5.7 | 153 | 152 |
| | 21 days* | 12.6 | 10.6 | 305 | 303 | 5.6 | 5.7 | 153 | 153 |
| F8 | Initial | 7.0 | 6.1 | 295 | 290 | 5.9 | 6.0 | 151 | 151 |
| | 1 day | 6.7 | 7.3 | 290 | 287 | 5.9 | 6.0 | 151 | 150 |
| | 5 days | 6.9 | 7.6 | 289 | 286 | 5.9 | 6.0 | 150 | 150 |
| | 7 days | 6.5 | 7.1 | 293 | 297 | 6.0 | 6.0 | 150 | 151 |
| | 14 days | 6.5 | 6.6 | 294 | 289 | 6.0 | 6.0 | 151 | 149 |
| | 21 days | 7.0 | 6.5 | 293 | 292 | 6.0 | 6.0 | 150 | 150 |
| | 21 days* | 7.5 | 6.3 | 291 | 288 | 6.0 | 6.0 | 151 | 147 |
| F9 | Initial | 13.9 | 12.4 | 298 | 291 | 5.9 | 6.0 | 149 | 149 |
| | 1 day | 12.5 | 12.7 | 292 | 293 | 5.9 | 6.0 | 151 | 148 |
| | 5 days | 13.6 | 13.5 | 291 | 292 | 5.9 | 6.0 | 150 | 148 |
| | 7 days | 12.8 | 13.7 | 295 | 296 | 6.0 | 6.0 | 150 | 153 |
| | 14 days | 12.7 | 12.3 | 295 | 294 | 6.0 | 6.0 | 149 | 150 |
| | 21 days | 13.1 | 12.9 | 295 | 296 | 6.0 | 6.0 | 150 | 150 |
| | 21 days* | 13.8 | 12.4 | 294 | 297 | 6.0 | 6.0 | 152 | 149 |
| F10 | Initial | 7.1 | 6.3 | 301 | 295 | 6.0 | 6.0 | 148 | 149 |
| | 1 day | 6.7 | 6.5 | 295 | 293 | 5.9 | 6.0 | 149 | 148 |
| | 5 days | 11.1 | 8.3 | 292 | 291 | 5.9 | 6.0 | 146 | 147 |
| | 7 days | 6.5 | 7.5 | 296 | 296 | 6.0 | 6.0 | 149 | 148 |
| | 14 days | 6.1 | 6.1 | 297 | 298 | 6.0 | 6.0 | 151 | 148 |
| | 21 days | 6.6 | 6.5 | 295 | 295 | 6.0 | 6.0 | 149 | 149 |
| | 21 days* | 7.4 | 5.9 | 295 | 295 | 6.0 | 6.0 | 150 | 148 |
| F11 | Initial | 4.9 | 3.3 | 306 | 298 | 5.6 | 5.6 | 148 | 146 |
| | 1 day | 3.8 | 4.0 | 299 | 300 | 5.6 | 5.7 | 147 | 144 |
| | 5 days | 4.9 | 4.2 | 300 | 297 | 5.6 | 5.7 | 145 | 147 |
| | 7 days | 3.7 | 4.2 | 300 | 300 | 5.7 | 5.7 | 147 | 146 |
| | 14 days | 3.7 | 3.2 | 301 | 301 | 5.7 | 5.7 | 147 | 148 |
| | 21 days | 3.7 | 3.3 | 303 | 301 | 5.6 | 5.7 | 148 | 146 |
| | 21 days* | 5.2 | 3.2 | 302 | 298 | 5.6 | 5.7 | 146 | 147 |

*corresponds to no movement, whereas the other samples were shaken for the indicated amount of time.

### Binding Activity

The results of the SPR (Biacore) measurements showed that the shaking does not influence the binding activity of the molecule for the syringes on a rocking shaker as well as for the vials on a horizontal shaker. Overall, the binding activity remained high in a range of 91-11100 and the specific binding activity in a range of 98-107%.

### Results and Discussion

The opalescence depends on the formulation composition and ranges from 5 FNU in an excipient-free or buffer-free respectively to 14 FNU, but did not significantly increase over time.

The pH value, osmolality, opalescence and protein concentrations as well as binding activity against IL-23 remained unchanged for all formulations over the entire study period.

### 5.4. Further Analytics and Results

In addition, further analytics were performed for the eleven formulations tested with the following results. Shaking type and times, storage and used syringes and vials were as described above.
The TIP-SEC fragments contents remained essentially constant over the shaking time in vials and syringes. The fragments content was in a range of 0.3-0.5%. The UP-SEC LMW contents remained essentially constant over the shaking time in vials and syringes. The fragments content was in a range of 1.3-1.4%.
The weak cation exchange chromatography (WCX) showed that the percentage of the distribution of the main peak, APG and BPG remained at a constant level for all formulations tested during the study. The levels of the main peak, APG and BPG did not change significantly over shaking time. No differences between the formulations were observed.
The particle content measured by Micro Flow Imaging (MFI) remained essentially constant over the 21 days shaking time.

### 5.5. Summary

All in all, only minor differences in stability-indicating parameters were detected between formulations following exposure to shaking stress. For example, the formulations F3 and F7 showed highest monomer content (HP-SEC & UP-SEC) but they also showed the highest level of opalescence. It can be summarized that the formulations being tested in this study would be a viable formulation.

6. Comparison of Acetate and Succinate Buffer Systems with Different pH Values in Multiple Freeze/Thaw Cycles

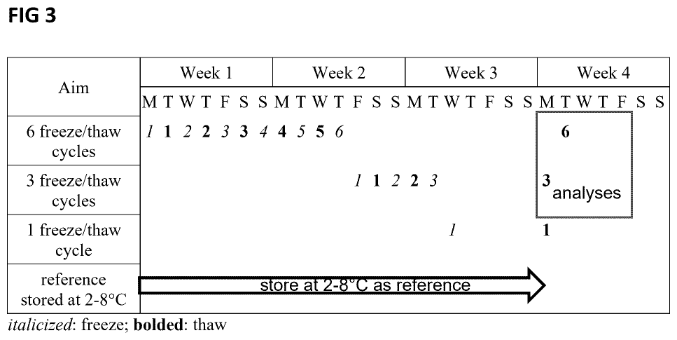

The freeze and thaw behavior of different formulations at 150 mg/mL risankizumab and its influence on product quality was evaluated. Therefore, the formulations were exposed to freezing and thawing stresses in mini bags at the intended target concentration of 150 mg/mL and a fill volume of 12 mL to simulate the conditions of bag-freezing in pilot or large scale. An overview of the experimental schedule is provided in FIG. 3.

In total eleven formulations that differ in pH, buffering strategy and tonicity agent plus an intermediate storage bulk were filled into mini-bags with a filling volume of 12 mL followed by a controlled freezing step to −40° C. Additionally bags were stored at 5° C. Note, the eleven formulations correspond to the ones tested in the previous example.

The freezing step was performed using a freeze-thaw setup provided by a classical freeze-dryer. Here, the mini-bags with a sample volume of 12 mL were frozen controlled with a freezing ramp of 0.5° C./min to −40° C. At this point the temperature remained constant over sixteen hours to ensure complete freezing of the sample volume. The thawing step was performed according to the freezing step with a heating rate of 0.5° C./min. The hold time at room temperature was set to four hours.

A complete freeze/thaw cycle (1×F/T) is defined as follows:
1. Freezing from room temperature to −40° (0.5° C./min)
2. Hold time of 16 hours at −40° C.
3. Thawing from −40° C. to room temperature (0.5° C./min)
4. Hold time of 4 hours at room temperature

This procedure was conducted for 1×F/T, 3×F/T and 6×F/T. After completion of the final process cycle the bags were transferred into a freezer at −40° C. and stored until the samples were thawed and analyzed simultaneously.

### 6.1. Preparation of Formulations

The formulations were prepared as described in the prior example (see Table 21). A further formulation F12 was prepared which is excipient-free containing 0.02% PS20, pH 5.7. 12 mL sterile filtered protein solutions were filled under laminar flow into the sterilized primary packaging materials, which are Mini Flexboy bags having a volume of 15 mL. All bags were inspected for visual particles and results were recorded.

In each freeze-thaw run twelve bags were put on each plate within the freeze-dryer. In total 36 bags were freeze/thawed each run including three bags per formulation. The bags were distributed in a defined scheme to eliminate influences of the bag position within the freeze-dryer.

### 6.2. Analytics

After conducting all cycles according to the experimental plan above, the bags were thawed in an additional thawing step successively. This procedure had the advantage that the samples could be analyzed altogether. The bags were transferred into the precooled freeze-dryer at −40° C. followed by the thawing step. HP-SEC, UP-SEC, osmolality, pH, protein concentration, opalescence, binding activity and sub-visible particle measurements were performed.

Following devices were used for analysis:
UV-Vis spectrophotometer Solo VPE: Conc. at 280 nm, baseline correction at 320 nm, extinction coefficient: 1.52; C Technologies, Inc., NJ, USA
Opalescence: HACH Lange opalescence meter; filter: 400-600 nm; Hach Lange GmbH, Dusseldorf, Germany
Ultra performance size exclusion chromatography (UP-SEC): UPLC26, H-Class UV-detection at 280 nm Waters, Milford, MA
Charge heterogeneity by weak cation exchange chromatography (WCX): HPLC75; Fluorescence detection Extinction: 278 nm, Emission: 350 nm; Waters, Milford, MA
IL-23 binding activity: Biacore T200 Chip: CM5 GE Healthcare, Chalfont St Giles, UK
pH-meter: SevenGo—Mettler Toledo, Columbus, OH
Particel sizer: Micro Flow Imaging™ Flow Microscope; By micro flow imaging (MFI); Brightwell Technologies Inc, Ottawa, ON, Canada
Osmometer: Osmomat 030 By freezing point depression, Gonotec GmbH, Berlin, Germany

Further details on the utilized analysis methods are described below.

### 6.3. Results

### 6.3.1. Measurement of the Monomer and HMW Content

Monomer content is a key quality attribute of protein stability and quality during stress-induced storage. HP-SEC and UP-SEC was used to determine levels of aggregate formation during freeze/thaw of the mini-bags. The subsequent table summarizes the results of the HP-SEC & UP-SEC analytic.

TABLE 25
HP-SEC / UP-SEC: Initial values and values after 1 / 3 / 6 F/T cycles and three
weeks at 5° C. in %.

| | | Aggregate | Monomer | Fragment | Aggregate | Monomer | Fragment |
|---|---|---|---|---|---|---|---|
| | Sampling | HP-SEC | HP-SEC | HP-SEC | UP-SEC | UP-SEC | UP-SEC |
| Formulation | time point | % | % | % | % | % | % |
| F1 | intial | 1.4 | 98.3 | 0.4 | 1.3 | 97.2 | 1.5 |
| | 1 × F/T | 1.5 | 98.1 | 0.4 | 1.4 | 97.3 | 1.3 |
| | 3 × F/T | 1.5 | 98.2 | 0 4 | 1.4 | 97.4 | 1.2 |
| | 6 × F/T | 1.5 | 98.1 | 0.4 | 1.4 | 97.4 | 1.3 |
| | 5° C. | 1.6 | 98.1 | 0.4 | 1.4 | 97.3 | 1.3 |
| F2 | inital | 1.4 | 98.3 | 0.3 | 1.3 | 97.2 | 1.5 |
| | 1 × F/T | 1.5 | 98.1 | 0.4 | 1.5 | 97.2 | 1.5 |
| | 3 × F/T | 1.4 | 98.2 | 0.4 | 1.5 | 97.2 | 1.3 |
| | 6 × F/T | 1.5 | 98.2 | 0.4 | 1.4 | 97.3 | 1.3 |
| | 5° C. | 1.5 | 98.1 | 0.4 | 1.4 | 97.3 | 1.3 |
| F3 | inital | 1.2 | 98.4 | 0.3 | 1.2 | 97.3 | 1.5 |
| | 1 × F/T | 1.3 | 98.3 | 0.4 | 1.3 | 97.4 | 1.3 |
| | 3 × F/T | 1.3 | 98.4 | 0.4 | 1.3 | 97.5 | 1.3 |
| | 6 × F/T | 1.4 | 98.3 | 0.4 | 1.3 | 97.5 | 1.3 |
| | 5° C. | 1.4 | 98.3 | 0.4 | 1.3 | 97.5 | 1.3 |
| F4 | inital | 1.6 | 99.0 | 0.3 | 1.8 | 96.8 | 1.5 |
| | 1 × F/T | 2.1 | 97.6 | 0.4 | 2.0 | 96.8 | 1.2 |
| | 3 × F/T | 1.9 | 97.7 | 0.4 | 1.9 | 96.8 | 1.3 |
| | 6 × F/T | 2.1 | 97.5 | 0.4 | 1.9 | 96.8 | 1.3 |
| | 5° C. | 2.0 | 97.6 | 0.4 | 1.9 | 96.9 | 1.3 |
| F5 | inital | 1.6 | 98.0 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.2 | 0.4 | 1.4 | 97.3 | 1.3 |
| | 5° C. | 1.5 | 98.2 | 0.4 | 1.4 | 97.3 | 1.3 |
| F6 | inital | 1.3 | 98.3 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.2 | 0.4 | 1.4 | 97.3 | 1.3 |
| | 5° C. | 1.5 | 98.1 | 0.4 | 1.4 | 97.3 | 1.3 |
| F7 | inital | 1.3 | 98.4 | 0.3 | 1.2 | 97.3 | 1.5 |
| | 1 × F/T | 1.4 | 98.3 | 0.4 | 1.3 | 97.4 | 1.3 |
| | 3 × F/T | 1.3 | 98.3 | 0.4 | 1.3 | 97.5 | 1.3 |
| | 6 × F/T | 1.4 | 98.3 | 0.4 | 1.2 | 97.5 | 1.3 |
| | 5° C. | 1.4 | 98.3 | 0.4 | 1.3 | 97.5 | 1.3 |
| F8 | inital | 1.5 | 98.2 | 0.3 | 1.5 | 97.0 | 1.4 |
| | 1 × F/T | 1.8 | 97.8 | 0.4 | 1.7 | 97.0 | 1.3 |
| | 3 × F/T | 1.7 | 97.9 | 0.4 | 1.6 | 97.1 | 1.3 |
| | 6 × F/T | 1.7 | 97.9 | 0.4 | 1.6 | 97.1 | 1.3 |
| | 5° C. | 1.7 | 97.9 | 0.4 | 1.6 | 97.1 | 1.3 |
| F9 | inital | 1.5 | 98.2 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.1 | 0.4 | 1.4 | 97.3 | 1.3 |
| | 5° C. | 1.5 | 98.1 | 0.4 | 1.4 | 97.3 | 1.3 |
| F10 | inital | 1.6 | 98.1 | 0.3 | 1.5 | 97.0 | 1.5 |
| | 1 × F/T | 1.7 | 97.9 | 0.4 | 1.6 | 97.1 | 1.3 |
| | 3 × F/T | 1.7 | 98.0 | 0.4 | 1.6 | 97.1 | 1.3 |
| | 6 × F/T | 1.7 | 98.0 | 0.4 | 1.6 | 97.1 | 1.3 |
| | 5° C. | 1.7 | 97.9 | 0.4 | 1.5 | 97.3 | 1.3 |
| F11 | inital | 1.4 | 98.3 | 0.3 | 1.3 | 97.3 | 1.5 |
| | 1 × F/T | 1.4 | 98.2 | 0.4 | 1.4 | 97.3 | 1.3 |
| | 3 × F/T | 1.4 | 98.2 | 0.4 | 1.4 | 97.3 | 1.3 |
| | 6 × F/T | 1.4 | 98.2 | 0.4 | 1.4 | 97.3 | 1.3 |
| | 5° C. | 1.4 | 98.2 | 0.4 | 1.4 | 97.3 | 1.3 |
| F12 | inital | 1.5 | 98.2 | 0.3 | 1.3 | 97.2 | 1.5 |
| | 1 × F/T | 1.7 | 98.0 | 0.4 | 1.6 | 97.1 | 1.3 |
| | 3 × F/T | 1.8 | 97.8 | 0.4 | 1.7 | 97.0 | 1.3 |
| | 6 × F/T | 2.0 | 97.6 | 0.4 | 1.9 | 96.8 | 1.3 |
| | 5° C. | 1.5 | 98.1 | 0.4 | 1.3 | 97.4 | 1.3 |

### Results and Discussion

The data show that the formation of aggregates is mainly driven by the pH. By comparing the results after six FIT cycles it is obvious that formulations at pH 6.0 or 6.2 show a slightly increased HMW content of 0.2-0.6 Oo in comparison to solutions formulated at pH 5.7. Formulations containing L-arginine like F3 and F7 showed the lowest level of aggregation after six FIT cycles. Generally, it can be summarized that the freeze/thaw stress does not significantly increase the HMW content in comparison to the results after 21 days at 5° C.

### 6.3.2. Measurement of the Opalescence and Further Parameters

Opalescence, osmolality, pH-values and protein concentrations of all tested formulations remained unchanged following 6 F/T cycles and after storage at 5° C. for three weeks. The lowest level of opalescence was observed in buffer-free formulations (F11 & F12).

TABLE 26
Initial values and values after 1 / 3 / 6 F/T cycles of opalescence in FNU, osmolality in
mOsm/kg, pH-values and protein conc. in g/L.

| | | Sampling | Opalescence | Osmolatity | | Protein conc. |
|---|---|---|---|---|---|---|
| Formulation | Components | time point | FNU | mOsmol · kg⁻¹ | pH | g · L⁻¹ |
| F1 | 10 mM acetate + | initial | 7.7 | 306 | 5.6 | 153 |
| | 6.5 mM succinate + | 1 × F/T | 8.0 | 307 | 5.6 | 150 |
| | 185 mM sorbitol + | 3 × F/T | 8.1 | 308 | 5.7 | 151 |
| | 0.02% PS20, | 6 × F/T | 8.1 | 307 | 5.7 | 151 |
| | pH 5.7 | 5° C. | 8.8 | 306 | 5.7 | 151 |
| F2 | 10 mM acetate + | initial | 4.8 | 304 | 5.7 | 153 |
| | 200 mM sorbitol + | 1 × F/T | 5.2 | 305 | 5.6 | 152 |
| | 0.02% PS20, | 3 × F/T | 4.8 | 306 | 5.6 | 153 |
| | pH 5.7 | 6 × F/T | 5.4 | 306 | 5.7 | 152 |
| | | 5° C. | 5.6 | 306 | 5.7 | 150 |
| F3 | 10 mM acetate + | initial | 11.1 | 292 | 5.7 | 153 |
| | 50 mM L-arginie | 1 × F/T | 11.8 | 292 | 5.6 | 152 |
| | HCl + 110 mM | 3 × F/T | 12.1 | 292 | 5.6 | 152 |
| | sorbitol + 0.02% | 6 × F/T | 12.2 | 251 | 5.6 | 153 |
| | PS20, pH 5.7 | 5° C. | 11.5 | 292 | 5.6 | 150 |
| F4 | 10 mM succinate + | initial | 10.6 | 306 | 6.1 | 154 |
| | 185 mM sorbitol + | 1 × F/T | 11.1 | 296 | 6.1 | 150 |
| | 0.02% PS20, | 3 × F/T | 12.1 | 300 | 6.1 | 151 |
| | pH 6.2 | 6 × F/T | 11.3 | 301 | 6.1 | 151 |
| | | 5° C. | 11.3 | 300 | 6.1 | 151 |
| F5 | 10 mM acetate + | initial | 4.7 | 307 | 5.6 | 154 |
| | 95 mM mannitol + | 1 × F/T | 4.9 | 302 | 5.6 | 151 |
| | 95 mM trehalose + | 3 × F/T | 5.4 | 305 | 5.6 | 153 |
| | 0.02% PS20, | 6 × F/T | 5.1 | 305 | 5.6 | 153 |
| | pH 5.7 | 5° C. | 4.9 | 306 | 5.6 | 150 |
| F6 | 10 mM acetate + | initial | 4.5 | 309 | 5.0 | 152 |
| | 185 mM trehlaose + | 1 × F/T | 4.7 | 306 | 5.6 | 152 |
| | 0.02% PS20, | 3 × F/T | 5.0 | 109 | 5.6 | 152 |
| | pH 5.7 | 6 × F/T | 4.8 | 309 | 5.6 | 151 |
| | | 5° C. | 4.7 | 309 | 5.5 | 150 |
| F7 | 10 mM acetate + | initial | 10.6 | 307 | 5.7 | 152 |
| | 50 mM L-arginine | 1 × F/T | 11.2 | 303 | 5.6 | 151 |
| | HCl + 110 mM | 3 × F/T | 11.8 | 301 | 5.6 | 151 |
| | trehalose + 0.02% | 6 × F/T | 11.0 | 305 | 5.5 | 153 |
| | PS20, pH 5.7 | 5° C | 12.0 | 304 | 5.6 | 152 |
| F8 | 44 mM succinate + | initial | 6.2 | 300 | 5.9 | 148 |
| | 185 mM | 1 × F/T | 6.4 | 291 | 5.9 | 150 |
| | trehalose + | 3 × F/T | 6.7 | 256 | 5.9 | 151 |
| | 0.02% P520, | 6 × F/T | 6.3 | 295 | 5.9 | 152 |
| | pH 6.0 | 5° C. | 6.2 | 296 | 5.9 | 151 |
| F9 | 4.4 mM succinate + | initial | 11.9 | 303 | 5.9 | 153 |
| | 50 mM L | 1 × F/T | 12.4 | 294 | 5.9 | 151 |
| | arginine HCl + | 3 × F/T | 12.9 | 296 | 5.8 | 153 |
| | 110 mM trehalose + | 6 × F/T | 12.3 | 295 | 5.8 | 153 |
| | 0.02% PS20, | 5° C. | 11.8 | 295 | 5.9 | 150 |
| | pH 6.0 | | | | | |
| F10 | 4.4 mM succinate + | initial | 6.0 | 314 | 5.9 | 153 |
| | 95 mM mannitol + | 1 × F/T | 5.8 | 298 | 5.9 | 151 |
| | 95 mM trehalose + | 3 × F/T | 6.6 | 298 | 5.8 | 150 |
| | 0.02% PS20, | 6 × F/T | 6.1 | 298 | 5.8 | 153 |
| | pH 6.0 | 5° C. | 5.9 | 301 | 5.9 | 150 |
| F11 | Buffer-free, 200 | initial | 3.4 | 322 | 5.6 | 154 |
| | mM trehalose, | 1 × F/T | 3.3 | 305 | 5.6 | 150 |
| | 0.02% PS20, | 3 × F/T | 3.7 | 306 | 5.6 | 151 |
| | pH 5.7 | 6 × F/T | 3.6 | 305 | 5.7 | 153 |
| | | 5° C. | 3.6 | 306 | 5.5 | 152 |
| F12 | Excipient-free, | initial | 3.5 | 29 | 5.6 | 151 |
| | 0.02% PS20, | 1 × F/T | 3.8 | 25 | 5.6 | 152 |
| | pH 5.7 | 3 × F/T | 4.0 | 25 | 5.6 | 151 |
| | | 6 × F/T | 3.8 | 25 | 5.5 | 151 |
| | | 5° C. | 3.6 | 27 | 5.6 | 151 |

### Binding Activity

The results of the SPR (Biacore) measurements of the IL23 binding activity show that the freeze/thaw cycles do not influence the binding activity of the molecule. The binding activity ranges between 96-117%.

### Results and Discussion

The measured opalescence depended on the formulation. The pH value, osmolality, opalescence and protein concentrations as well as binding of IL-23 essentially remained unchanged and thus stable for all formulations over the entire investigation period regardless of the stress condition (FIT & hold time at 5° C.).

### 6.3.3. Measurement of Particles

The subsequent table summarizes the number of particles for each STP. No clear trends could be observed for all formulations being tested. The formulations F3, F7 and F9 show a slightly increased amount of SVP in comparison to other formulations being tested. This observation was mainly seen for the SVP ≥2 μm and >10 μm.

TABLE 27
Subvisible Particles - MFI: Initial values and values after one, three and six F/T
cycles and three weeks at 5° C.

| | Particle | Number of measured particles | | | | | | | | | | | |
|---|---|---|---|---|---|---|---|---|---|---|---|---|---|
| Treatment | size | F1 | F2 | F3 | F4 | F5 | F6 | F7 | F8 | F9 | F10 | F11 | F12 |
| Initial | ≥2 μm | 505 | 1104 | 1604 | 723 | 508 | 1046 | 878 | 1259 | 622 | 1255 | 1314 | 673 |
| | ≥10 μm | 44 | 32 | 50 | 34 | 31 | 38 | 18 | 71 | 19 | 38 | 41 | 36 |
| | ≥25 μm | 2 | 2 | 2 | 2 | 2 | 3 | 2 | 7 | 2 | 1 | 2 | 2 |
| 1× | ≥2 μm | 1443 | 660 | 2426 | 831 | 710 | 883 | 2784 | 1260 | 2557 | 1070 | 925 | 1759 |
| F/T | ≥10 μm | 49 | 19 | 639 | 18 | 38 | 21 | 683 | 28 | 995 | 45 | 35 | 63 |
| | ≥25 μm | 1 | 0 | 6 | 2 | 5 | 2 | 1 | 2 | 8 | 4 | 1 | 2 |
| 3× | ≥2 μm | 1485 | 836 | 2764 | 1440 | 717 | 656 | 3501 | 698 | 2708 | 901 | 520 | 1513 |
| F/T | ≥10 μm | 79 | 42 | 898 | 56 | 49 | 44 | 604 | 34 | 950 | 60 | 30 | 59 |
| | ≥25 μm | 6 | 6 | 10 | 3 | 2 | 3 | 10 | 5 | 3 | 5 | 5 | 3 |
| 6× | ≥2 μm | 1392 | 1008 | 4551 | 1214 | 714 | 1121 | 3265 | 1051 | 2752 | 810 | 925 | 1868 |
| F/T | ≥10 μm | 81 | 41 | 1089 | 31 | 48 | 69 | 518 | 38 | 1209 | 60 | 43 | 54 |
| | ≥25 μm | 7 | 10 | 41 | 4 | 9 | 6 | 17 | 1 | 5 | 7 | 3 | 3 |
| 5° C. | ≥2 μm | 1780 | 1259 | 2222 | 1341 | 555 | 784 | 2148 | 879 | 2825 | 1343 | 983 | 2131 |
| | ≥10 μm | 121 | 37 | 439 | 54 | 32 | 35 | 599 | 17 | 815 | 50 | 38 | 80 |
| | ≥25 μm | 4 | 1 | 6 | 6 | 3 | 4 | 2 | 1 | 2 | 6 | 3 | 7 |

### Results and Discussion

While for ≥2 μm particles similar trends are observed, a slight increase of particles having a size of ≥10 μm was observed for F3, F7 and F9 compared to others. For ≥25 μm particles, a slight increase for F3 after 6×F/T was observed. Overall, particle formation was no major issue during F/T for all tested formulations.

### 6.4. Further Analytics and Results

In addition, further analytics were performed for the twelve formulations tested with the following results (freeze/thaw cycles were as described above).
The weak cation exchange chromatography (WCX) showed that the percentage of the distribution of the main peak, APG and BPG remained at a constant level for all formulations tested during the study. The levels of the main peak, APG and BPG did not change significantly over 6 freeze/thaw cycles. The main peak ranged from 65-6700, the APG content from 2123 the BPG content from about 11-14%. No differences between the formulations were observed.

### 6.5. Summary of Results

The results can be summarized as follows:
Visual inspection: After six F/T cycles, no observations could be made during visual inspection for all formulations.
SVP: Regarding the sub-visible particle level no major issues could be observed. There was a slight increase for F3, F7 and F9 compared to other formulations but significantly below specifications of Pharmacopeia.
HP-SEC & UP-SEC: For the test methods with a focus on protein integrity like HP-SEC and UP-SEC F3 turned out to be the most stable formulation and F4 as the least stable. F12 without any buffer or excipients exhibited an acceptable stability during freeze/thaw cycles.
IEC: For the results of the IEC no discrimination for any formulation could be observed. The F/T-cycles do not negatively influence the contribution of APG and BPG.
Opalescence: The opalescence depends on the formulation and ranges from 4 FNU in an excipient-free or buffer-free formulation to 13 FNU.
The pH value, osmolality, opalescence and protein concentrations as well as binding remained unchanged for all formulations over the entire investigation period regardless of the stress condition (F/T & hold time at 5° C.).

It can be summarized that most of the formulations being tested in this example are viable formulations for a 150 mg/mL formulation. Only minor effects of the freeze/thaw stress on protein stability were observed. Due to medical concern with formulations containing sorbitol, these formulations may be found less advantageous in order to also address fructose-intolerant patients. Nevertheless, for other patients sorbitol-containing solutions may be found useful. All in all, only minor differences in stability-indicating parameters could be detected between formulations following exposure to F/T. For example the formulation F3 was most stable in monomer content (HP-SEC) but contrarily an increased level of subvisible particles could be detected.

### 7. Impact of the pH on the Formulation Stability

The impact of the pH value on the stability of 150 mg/mL risankizumab formulations were tested with the formulations shown in Table 28.

TABLE 28
Composition of formulations.

| Formulation | pH | Acetate | Trehalose | Polysorbate 20 |
|---|---|---|---|---|
| F1 | 5.0 | 10 mM | 185 mM | 0.2 mg/mL |
| F2 | 5.2 | | | |
| F3 | 5.5 | | | |
| F4 | 5.7 | | | |
| F5 | 5.9 | | | |
| F6 | 6.2 | | | |

### 7.1. Preparation of Formulations

The formulations were prepared as described above.

### 7.2. Analytics

Measurements of the samples were performed at 1, 3, 6, 9, 12, 18, 24 and 36 months storage, as well as initially before storage. Various methods for analysis were used, including HIC, UP-SEC, IEC, as well as viscosity, break loose and gliding force and binding specificity measurements. Further details on the utilized analysis methods are described below.

### 7.3. Results

### 7.3.1. Measurement of the Monomer Content

The monomer content was measured as in the previous examples using UP-SEC analysis, the results are shown in Table 29.

TABLE 29
UP-SEC-Monomer-measurements in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 98.3 | 98.3 | 98.2 | 98.0 | 97.8 | 97.3 |
| 5° C. | 3 | 98.1 | 98.1 | 97.9 | 97.7 | 97.4 | 96.8 |
| 5° C. | 6 | 98.0 | 98.0 | 97.8 | 97.6 | 97.4 | 96.9 |
| 5° C. | 9 | 97.5 | 97.5 | 97.3 | 97.2 | 97.0 | 96.5 |
| 5° C. | 12 | 97.5 | 97.5 | 97.4 | 97.2 | 97.0 | 96.5 |
| 5° C. | 18 | 97.6 | 97.6 | 97.5 | 97.4 | 97.2 | 96.8 |
| 5° C. | 24 | 97.3 | 97.4 | 97.3 | 97.2 | 97.0 | 96.6 |
| 5° C. | 36 | 97.1 | 97.0 | 97.0 | 96.9 | 96.8 | 96.0 |
| 25° C. | 1 | 97.6 | 97.6 | 97.4 | 97.2 | 97.0 | 96.4 |
| 25° C. | 3 | 96.4 | 96.6 | 96.5 | 96.4 | 96.1 | 95.7 |
| 25° C. | 6 | 95.1 | 95.4 | 95.7 | 95.7 | 95.5 | 95.1 |
| 25° C. | 9 | 93.4 | 94.0 | 94.4 | 94.5 | 94.4 | 94.0 |
| 25° C. | 12 | 92.3 | 93.1 | 93.8 | 93.9 | 93.9 | 93.5 |
| 40° C. | 1 | 94.2 | 94.6 | 94.9 | 94.8 | 94.7 | 94.3 |
| 40° C. | 3 | 87.6 | 89.0 | 90.3 | 90.6 | 90.8 | 90.7 |

### Results and Discussion

The monomer content measurements show that the tested formulations were stable over the tested range of pH values from pH 5.0 to 6.2. Therefore, a broad range of pH values is applicable in order to obtain high stability formulations of 150 mg/mL risankizumab. High monomer values were obtained for pH values around 5.7, whereas relatively low monomer contents were measured for more acidic conditions of about pH 5.0 (see for instance last measurement points of pH 5.0 at 25° C. or 40° C.). Hence, formulations of high concentrations of risankizumab (here 150 mg/mL) having a pH of about 5.7 proved to be particularly advantageous, in particular in the provided formulations of the present example.

### 7.3.2. Measurement of the HMW Content

Also the HMW content of the formulations was determined using UP-SEC, whereby following results were obtained:

TABLE 30
UP-SEC-HMW measurements in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 0.8 | 0.9 | 1.1 | 1.2 | 1.4 | 1.9 |
| 5° C. | 3 | 1.0 | 1.1 | 1.3 | 1.4 | 1.8 | 2.4 |
| 5° C. | 6 | 1.1 | 1.2 | 1.4 | 1.6 | 1.8 | 2.3 |
| 5° C. | 9 | 1.3 | 1.4 | 1.6 | 1.7 | 1.9 | 2.4 |
| 5° C. | 12 | 1.4 | 1.4 | 1.6 | 1.7 | 2.0 | 2.4 |
| 5° C. | 18 | 1.4 | 1.5 | 1.6 | 1.7 | 1.9 | 2.3 |
| 5° C. | 24 | 1.8 | 1.8 | 1.9 | 2.1 | 2.3 | 2.7 |
| 5° C. | 36 | 1.8 | 1.9 | 2.0 | 2.1 | 2.3 | 2.8 |
| 25° C. | 1 | 1.3 | 1.4 | 1.6 | 1.8 | 2.1 | 2.7 |
| 25° C. | 3 | 1.7 | 1.8 | 2.1 | 2.3 | 2.6 | 3.0 |
| 25° C. | 6 | 2.1 | 2.2 | 2.3 | 2.4 | 2.6 | 3.1 |
| 25° C. | 9 | 2.6 | 2.6 | 2.9 | 2.8 | 3.0 | 3.4 |
| 25° C. | 12 | 2.9 | 2.9 | 2.8 | 3.0 | 3.1 | 3.5 |
| 40° C. | 1 | 2.5 | 2.5 | 2.7 | 2.9 | 3.1 | 3.6 |
| 40° C. | 3 | 4.9 | 4.5 | 4.3 | 4.5 | 4.6 | 4.8 |

### Results and Discussion

The HMW content correlates with the monomer measurements. Overall, the tested formulations were stable over a range of pH values. Particular low increases of the HMW contents were obtained for pH values around 5.7. However, higher pH values (e.g. a pH of 6.2) appear to have led to slightly higher HMW values.

### 7.3.3. Measurement of the LMW Content

The LMW content was measured by UP-SEC analysis, which revealed following results:

TABLE 31
UP-SEC-LMW-measurements in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 0.8 | 0.8 | 0.8 | 0.8 | 0.8 | 0.8 |
| 5° C. | 3 | 0.9 | 0.8 | 0.8 | 0.8 | 0.8 | 0.8 |
| 5° C. | 6 | 0.9 | 0.8 | 0.9 | 0.9 | 0.8 | 0.8 |
| 5° C. | 9 | 1.2 | 1.2 | 1.1 | 1.1 | 1.1 | 1.1 |
| 5° C. | 12 | 1.1 | 1.1 | 1.1 | 1.0 | 1.0 | 1.0 |
| 5° C. | 18 | 1.0 | 0.9 | 0.9 | 0.9 | 0.9 | 0.9 |
| 5° C. | 24 | 0.9 | 0.8 | 0.8 | 0.7 | 0.7 | 0.7 |
| 5° C. | 36 | 1.1 | 1.1 | 1.0 | 1.0 | 0.9 | 1.2 |
| 25° C. | 1 | 1.2 | 1.1 | 1.0 | 1.0 | 1.0 | 1.0 |
| 25° C. | 3 | 1.8 | 1.6 | 1.4 | 1.4 | 1.4 | 1.3 |
| 25° C. | 6 | 2.8 | 2.4 | 2.0 | 1.9 | 1.9 | 1.8 |
| 25° C. | 9 | 4.1 | 3.5 | 2.7 | 2.8 | 2.7 | 2.6 |
| 25° C. | 12 | 4.8 | 4.1 | 3.4 | 3.1 | 3.0 | 3.0 |
| 40° C. | 1 | 3.3 | 2.9 | 2.5 | 2.3 | 2.2 | 2.1 |
| 40° C. | 3 | 7.5 | 6.5 | 5.4 | 5.0 | 4.7 | 4.5 |

### Results and Discussion

The LMW content correlates with the monomer measurements. Particular low increases of the LMW contents were obtained for pH values around 5.7. However, lower pH values appear to have led to slightly higher LMW values. Overall, the tested formulations were stable over a range of 0 pH values.

### 7.3.4. Measurement of Species by Ion Exchange Chromatography (IEC)

Measurement of the ionic species was performed by ionic exchange chromatography. The results were then sorted into the main peak, acidic peak groups (APG) and basic peak groups (BPG).

TABLE 32
IEC measurements of the main peak in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 72 | 71 | 71 | 72 | 72 | 71 |
| 5° C. | 3 | 71 | 71 | 71 | 71 | 70 | 70 |
| 5° C. | 6 | 70 | 70 | 70 | 70 | 70 | 70 |
| 5° C. | 12 | 68 | 70 | 71 | 71 | 71 | 71 |
| 5° C. | 18 | 67 | 69 | 70 | 70 | 71 | 70 |
| 5° C. | 24 | 65 | 67 | 68 | 69 | 69 | 68 |
| 5° C. | 36 | 65 | 66 | 68 | 69 | 69 | 69 |
| 25° C. | 1 | 68 | 68 | 69 | 69 | 69 | 69 |
| 25° C. | 3 | 61 | 62 | 63 | 64 | 64 | 65 |
| 25° C. | 6 | 52 | 54 | 57 | 58 | 59 | 60 |
| 25° C. | 12 | 39 | 43 | 48 | 50 | 51 | 53 |
| 40° C. | 1 | 46 | 48 | 50 | 52 | 54 | 55 |
| 40° C. | 3 | 25 | 26 | 28 | 31 | 33 | 35 |

TABLE 33
IEC measurements of APG in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 22 | 22 | 22 | 22 | 22 | 22 |
| 5° C. | 3 | 22 | 22 | 23 | 23 | 23 | 23 |
| 5° C. | 6 | 23 | 23 | 23 | 23 | 23 | 23 |
| 5° C. | 12 | 20 | 20 | 20 | 20 | 21 | 21 |
| 5° C. | 18 | 22 | 22 | 23 | 23 | 23 | 23 |
| 5° C. | 24 | 20 | 21 | 21 | 21 | 22 | 22 |
| 5° C. | 36 | 21 | 22 | 22 | 22 | 23 | 23 |
| 25° C. | 1 | 23 | 24 | 24 | 24 | 24 | 24 |
| 25° C. | 3 | 27 | 28 | 28 | 27 | 28 | 27 |
| 25° C. | 6 | 32 | 33 | 33 | 33 | 32 | 32 |
| 25° C. | 12 | 34 | 37 | 39 | 39 | 38 | 38 |
| 40° C. | 1 | 37 | 38 | 39 | 38 | 37 | 37 |
| 40° C. | 3 | 55 | 59 | 61 | 60 | 59 | 58 |

TABLE 34
IEC measurements of BPG in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 6 | 6 | 7 | 6 | 7 | 7 |
| 5° C. | 3 | 7 | 7 | 7 | 7 | 7 | 7 |
| 5° C. | 6 | 7 | 7 | 7 | 7 | 7 | 7 |
| 5° C. | 12 | 12 | 10 | 9 | 9 | 8 | 8 |
| 5° C. | 18 | 11 | 9 | 8 | 7 | 7 | 6 |
| 5° C. | 24 | 15 | 13 | 11 | 10 | 9 | 9 |
| 5° C. | 36 | 15 | 12 | 10 | 9 | 8 | 8 |
| 25° C. | 1 | 9 | 8 | 8 | 7 | 8 | 8 |
| 25° C. | 3 | 12 | 11 | 9 | 9 | 8 | 8 |
| 25° C. | 6 | 16 | 13 | 10 | 9 | 9 | 8 |
| 25° C. | 12 | 27 | 20 | 14 | 11 | 10 | 9 |
| 40° C. | 1 | 17 | 14 | 11 | 10 | 9 | 9 |
| 40° C. | 3 | 20 | 15 | 10 | 9 | 8 | 7 |

### Results and Discussion

The IEC measurements show overall that the tested formulations were stable. All pH values led to high contents of the main peak. Noteworthy, an intermediate pH value of 5.7 and pH values around 5.7 showed a good compromise in comparison to the highest and lowest tested pH values, which showed an increase in APG or BPG species, respectively. Therefore, a pH of approximately 5.7 proved to be advantageous.

### 7.3.5. Measurement of Species by Hydrophobic Interaction Chromatography (HIC)

Measurement of the variants/subspecies of risankizumab was performed by hydrophobic interaction chromatography (HIC). The results were then sorted into the main peak, pre-peaks and post-peaks.

TABLE 35
HIC measurements of the main peak in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 96.8 | 96.7 | 96.7 | 96.7 | 96.4 | 96.0 |
| 5° C. | 3 | 96.7 | 96.7 | 96.7 | 96.6 | 96.3 | 95.8 |
| 5° C. | 6 | 96.7 | 96.7 | 96.7 | 96.5 | 96.5 | 95.9 |
| 5° C. | 12 | 97.2 | 97.2 | 97.1 | 97.1 | 96.9 | 96.6 |
| 5° C. | 18 | 96.0 | 96.1 | 96.2 | 96.1 | 95.9 | 95.6 |
| 5° C. | 24 | 96.7 | 96.7 | 96.9 | 96.8 | 96.7 | 96.5 |
| 5° C. | 36 | 95.3 | 95.4 | 95.7 | 95.7 | 95.7 | 95.4 |
| 25° C. | 1 | 96.2 | 96.1 | 96.1 | 95.9 | 95.7 | 95.3 |
| 25° C. | 3 | 95.4 | 95.3 | 95.4 | 95.4 | 95.1 | 94.7 |
| 25° C. | 6 | 94.7 | 95.0 | 95.1 | 95.1 | 95.1 | 94.8 |
| 25° C. | 12 | 93.8 | 94.1 | 94.6 | 94.7 | 94.7 | 94.4 |
| 40° C. | 1 | 93.6 | 94.2 | 94.3 | 94.2 | 94.2 | 93.6 |
| 40° C. | 3 | 88.5 | 89.8 | 90.4 | 90.8 | 90.7 | 90.7 |

TABLE 36
HIC measurements of pre-peaks in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 1.8 | 1.7 | 1.7 | 1.6 | 1.6 | 1.6 |
| 5° C. | 3 | 1.9 | 1.9 | 1.8 | 1.8 | 1.8 | 1.8 |
| 5° C. | 6 | 1.9 | 1.9 | 1.7 | 1.8 | 1.6 | 1.7 |
| 5° C. | 12 | 1.6 | 1.5 | 1.4 | 1.3 | 1.3 | 1.3 |
| 5° C. | 18 | 2.1 | 2.0 | 1.8 | 1.8 | 1.7 | 1.6 |
| 5° C. | 24 | 1.8 | 1.7 | 1.5 | 1.4 | 1.4 | 1.4 |
| 5° C. | 36 | 2.2 | 2.0 | 1.7 | 1.5 | 1.5 | 1.4 |
| 25° C. | 1 | 2.1 | 2.1 | 2.0 | 1.9 | 2.0 | 1.9 |
| 25° C. | 3 | 2.8 | 2.7 | 2.4 | 2.3 | 2.3 | 2.5 |
| 25° C. | 6 | 3.3 | 3.0 | 2.7 | 2.5 | 2.4 | 2.3 |
| 25° C. | 12 | 4.1 | 3.6 | 3.1 | 2.8 | 2.7 | 2.6 |
| 40° C. | 1 | 3.7 | 3.3 | 3.1 | 3.0 | 2.8 | 3.0 |
| 40° C. | 3 | 7.5 | 6.4 | 6.1 | 5.6 | 5.5 | 5.4 |

TABLE 37
HIC measurements of post-peaks in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 1.5 | 1.6 | 1.6 | 1.7 | 2.0 | 2.4 |
| 5° C. | 3 | 1.4 | 1.4 | 1.6 | 1.7 | 1.9 | 2.5 |
| 5° C. | 6 | 1.4 | 1.4 | 1.6 | 1.7 | 1.9 | 2.3 |
| 5° C. | 12 | 1.2 | 1.2 | 1.4 | 1.6 | 1.7 | 2.1 |
| 5° C. | 18 | 1.9 | 1.9 | 2.0 | 2.1 | 2.4 | 2.8 |
| 5° C. | 24 | 1.5 | 1.6 | 1.6 | 1.7 | 1.9 | 2.1 |
| 5° C. | 36 | 2.5 | 2.6 | 2.6 | 2.8 | 2.8 | 3.2 |
| 25° C. | 1 | 1.8 | 1.8 | 2.0 | 2.2 | 2.3 | 2.9 |
| 25° C. | 3 | 1.8 | 1.9 | 2.2 | 2.2 | 2.6 | 2.9 |
| 25° C. | 6 | 2.1 | 2.1 | 2.3 | 2.4 | 2.5 | 2.9 |
| 25° C. | 12 | 2.0 | 2.1 | 2.2 | 2.4 | 2.5 | 2.9 |
| 40° C. | 1 | 2.7 | 2.6 | 2.6 | 2.8 | 2.9 | 3.5 |
| 40° C. | 3 | 4.1 | 3.8 | 3.5 | 3.6 | 3.8 | 3.9 |

### Results and Discussion

The HIC measurements show overall that the tested formulations were stable. All pH values led to high contents of the main peak. Noteworthy, an intermediate pH value of 5.7 and pH values around 5.7 showed a good compromise in comparison to the highest and lowest tested pH values, which showed an increase in pre- and post-peaks, respectively.

### 7.3.6. Binding Activity

Measurement of the binding activity of risankizumab against IL-23 was performed using the Biacore T200. Following results were obtained:

TABLE 38
Binding activity in % of the formulations
having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 103 | 103 | 103 | 106 | 105 | 107 |
| 5° C. | 6 | 101 | 104 | 102 | 103 | 104 | 103 |
| 5° C. | 12 | 94 | 97 | 96 | 93 | 97 | 99 |
| 5° C. | 18 | 104 | 102 | 102 | 106 | 105 | 105 |
| 5° C. | 24 | 102 | 96 | 101 | 100 | 97 | 102 |
| 5° C. | 36 | 95 | 97 | 96 | 95 | 96 | 98 |
| 25° C. | 3 | 109 | 107 | 106 | 106 | 103 | 103 |
| 25° C. | 6 | 105 | 99 | 99 | 100 | 103 | 103 |
| 25° C. | 12 | 93 | 101 | 93 | 94 | 92 | 97 |
| 40° C. | 3 | 98 | 100 | 100 | 98 | 97 | 101 |

TABLE 39
Specific binding activity in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 100 | 100 | 100 | 100 | 100 | 100 |
| 5° C. | 6 | 100 | 99 | 99 | 99 | 99 | 99 |
| 5° C. | 12 | 99 | 100 | 100 | 100 | 100 | 100 |
| 5° C. | 18 | 99 | 99 | 99 | 100 | 100 | 100 |
| 5° C. | 24 | 99 | 99 | 99 | 99 | 99 | 99 |
| 5° C. | 36 | 100 | 100 | 100 | 100 | 100 | 100 |
| 25° C. | 3 | 99 | 99 | 99 | 99 | 99 | 99 |
| 25° C. | 6 | 98 | 98 | 99 | 98 | 99 | 98 |
| 25° C. | 12 | 98 | 98 | 98 | 98 | 98 | 98 |
| 40° C. | 3 | 96 | 96 | 97 | 97 | 98 | 98 |

### Results and Discussion

The binding activity measurement show overall high values of the tested formulations. Hence, the tested formulations stabilize risankizumab such that a high binding activity at a pH range from 5.0 to 6.2 was achieved.

### 7.3.7. Measurement of the Opalescence

Furthermore, the opalescence of the formulations was measured. The opalescence slightly changes over the time of analysis but remained overall highly constant ranging from 3 to 11. The results indicate that the formulations are overall stable. Noteworthy, lower pH values in general showed lower opalescence (3 to 6 FNU for pH 5.0) than higher pH values (7 to 11 FNU for pH 6.2). The intermediate pH of 5.7 had a opalescence ranging from 5 to 7 FNU, indicating that providing formulations having a pH of about 5.7 is advantageous, in particular in formulations according to the present example.

### 7.3.8. Measurement of the Viscosity and Syringe Gliding and Break Loose Forces

As further parameters, the viscosity and the syringe forces were measured, which include the average and maximal gliding force, as well as the break loose force. Following results were obtained:

TABLE 40
Measurement of the viscosity in mPas of the tested
formulations having different pH-values over time.

| | Storage | | | | | | |
|---|---|---|---|---|---|---|---|
| Storage | time, | | | | | | |
| condition | months | F1 | F2 | F3 | F4 | F5 | F6 |
| initial | 0 | 8.5 | 8.8 | 9.2 | 9.6 | 9.9 | 10.3 |
| 5° C. | 6 | 8.5 | 8.6 | 9.0 | 9.2 | 9.5 | 10.0 |
| 5° C. | 12 | 8.7 | 8.7 | 8.9 | 9.3 | 9.7 | 10.1 |
| 5° C. | 24 | 8.8 | 9.0 | 9.3 | 9.5 | 9.4 | 10.4 |
| 5° C. | 36 | 8.6 | 8.7 | 8.9 | 9.4 | 9.8 | 10.3 |
| 25° C. | 6 | 8.7 | 8.8 | 8.9 | 9.2 | 9.5 | 10.0 |
| 25° C. | 12 | 8.7 | 8.5 | 9.0 | 9.3 | 9.7 | 10.3 |

TABLE 41
Measurement of the maximal gliding force in N of the
tested formulations having different pH-values over time.

| Storage | Storage | | | | | | |
|---|---|---|---|---|---|---|---|
| condi- | time, | | | | | | |
| tion | months | F1 | F2 | F3 | F4 | F5 | F6 |
| initial | 0 | 6.3 | 5.9 | 6.0 | 6.6 | 6.0 | 7.1 |
| 5° C. | 3 | 6.4 | 6.9 | 6.3 | 6.5 | 6.5 | 7.0 |
| 5° C. | 6 | 8.4 | 7.1 | 6.3 | 6.3 | 6.2 | 6.7 |
| 5° C. | 9 | 7.7 | 8.5 | 6.4 | 6.3 | 7.1 | 6.9 |
| 5° C. | 12 | 9.4 | 7.6 | 6.6 | 6.9 | 7.3 | 6.8 |
| 5° C. | 18 | 9.2 | 10.4 | 8.4 | 6.5 | 6.9 | 7.0 |
| 5° C. | 24 | 9.0 | 8.2 | 7.7 | 7.7 | 6.9 | 6.6 |
| 5° C. | 36 | 9.2 | 8.3 | 7.7 | 7.7 | 6.9 | 7.1 |
| 25° C. | 1 | 7.0 | 8.6 | 7.3 | 6.4 | 6.0 | 5.6 |
| 25° C. | 3 | 11.8 | 10.0 | 8.5 | 7.3 | 8.3 | 6.8 |
| 25° C. | 6 | 12.4 | 11.9 | 11.0 | 11.2 | 8.4 | 7.2 |
| 25° C. | 9 | 12.9 | 13.3 | 13.2 | 10.7 | 11.1 | 8.4 |
| 25° C. | 12 | 13.6 | 16.7 | 13.7 | 11.9 | 12.5 | 10.2 |
| 40° C. | 1 | 10.8 | 10.3 | 11.8 | 8.7 | 7.9 | 7.2 |
| 40° C. | 3 | 18.1 | 20.9 | 20.6 | 18.0 | 17.1 | 13.4 |

TABLE 42
Measurement of the average gliding force in N of the
tested formulations having different pH-values over time.

| Storage | Storage | | | | | | |
|---|---|---|---|---|---|---|---|
| condi- | time, | | | | | | |
| tion | months | F1 | F2 | F3 | F4 | F5 | F6 |
| initial | 0 | 5.9 | 5.6 | 5.6 | 6.1 | 5.7 | 6.5 |
| 5° C. | 3 | 5.9 | 6.3 | 5.8 | 6.0 | 6.1 | 6.5 |
| 5° C. | 6 | 7.4 | 6.5 | 5.9 | 5.9 | 5.9 | 6.4 |
| 5° C. | 9 | 6.9 | 7.7 | 6.0 | 5.9 | 6.6 | 6.5 |
| 5° C. | 12 | 8.1 | 6.8 | 6.2 | 6.5 | 6.8 | 6.4 |
| 5° C. | 18 | 8.1 | 9.2 | 7.6 | 6.1 | 6.5 | 6.6 |
| 5° C. | 24 | 7.9 | 7.2 | 7.0 | 7.1 | 6.4 | 6.2 |
| 5° C. | 36 | 8.0 | 7.6 | 7.1 | 7.1 | 6.5 | 6.7 |
| 25° C. | 1 | 6.5 | 7.5 | 6.6 | 6.0 | 5.7 | 5.4 |
| 25° C. | 3 | 9.8 | 8.8 | 7.7 | 6.9 | 7.6 | 6.4 |
| 25° C. | 6 | 10.1 | 9.7 | 9.0 | 9.6 | 7.6 | 6.8 |
| 25° C. | 9 | 10.6 | 11.1 | 10.8 | 8.6 | 9.3 | 7.6 |
| 25° C. | 12 | 10.8 | 13.5 | 10.8 | 9.7 | 9.9 | 8.6 |
| 40° C. | 1 | 9.2 | 8.5 | 9.6 | 7.5 | 7.1 | 6.6 |
| 40° C. | 3 | 14.1 | 15.8 | 14.5 | 12.9 | 12.5 | 10.5 |

TABLE 43
Measurement of the break loose force in N of the tested
formulations having different pH-values over time.

| | Storage | | | | | | |
|---|---|---|---|---|---|---|---|
| Storage | time, | | | | | | |
| condition | months | F1 | F2 | F3 | F4 | F5 | F6 |
| initial | 0 | 3.9 | 3.9 | 4.1 | 4.1 | 4.1 | 4.1 |
| 5° C. | 3 | 4.9 | 4.9 | 5.2 | 5.1 | 5.1 | 5.0 |
| 5° C. | 6 | 4.4 | 4.4 | 4.5 | 4.6 | 4.6 | 4.6 |
| 5° C. | 9 | 4.0 | 4.0 | 4.0 | 4.2 | 4.1 | 4.1 |
| 5° C. | 12 | 4.7 | 4.6 | 4.6 | 4.6 | 4.7 | 4.7 |
| 5° C. | 18 | 4.7 | 4.6 | 4.7 | 4.8 | 4.8 | 4.9 |
| 5° C. | 24 | 4.6 | 4.4 | 4.6 | 4.6 | 4.6 | 4.9 |
| 5° C. | 36 | 4.5 | 4.5 | 4.7 | 4.7 | 4.7 | 4.6 |
| 25° C. | 1 | 4.4 | 4.6 | 4.7 | 5.0 | 4.6 | 4.4 |
| 25° C. | 3 | 5.5 | 5.5 | 5.7 | 5.7 | 5.7 | 5.5 |
| 25° C. | 6 | 5.4 | 5.3 | 5.2 | 5.4 | 5.2 | 5.4 |
| 25° C. | 9 | 5.4 | 5.2 | 5.3 | 5.0 | 5.0 | 5.0 |
| 25° C. | 12 | 5.4 | 5.3 | 5.3 | 5.3 | 5.3 | 5.4 |
| 40° C. | 1 | 5.4 | 5.3 | 5.4 | 5.5 | 5.4 | 5.3 |
| 40° C. | 3 | 7.0 | 6.7 | 6.7 | 6.2 | 6.5 | 6.1 |

### Results and Discussion

The viscosity measurements revealed a slightly higher viscosity for higher pH values. Thus lower pH values, such as pH 5.7, may be found advantageous in order to obtain formulations having a lower viscosity. Note, the mechanical measurements of the gliding and break loose force revealed overall very similar performance.

### 7.4. Further Analytics and Results

In addition, further analytics were performed for the six formulations tested with the following results. Storage times and temperatures were as described above.
The protein concentration remained essentially constant over the storage time of 36, 24, 12 and 3 months at the different storage temperatures tested, respectively 5° C., 25° C. and 40° C. Small deviations of protein concentration (145-155 mg/mL (24 months) and 145-158 mg/mL (36 months)) are due to analytical variations.
The pH value remained essentially constant over the storage time of 36, 24, 12 and 3 months at the different storage temperatures tested, respectively 5° C., 25° C. and 40° C. Measured pH values were thus in a range from 4.9-6.3.
The osmolality remained essentially constant over the storage time of 36, 24, 12 and 3 months at the different storage temperatures tested, respectively 5° C., 25° C. and 40° C. The tested values ranged from 301-323 mOsm/kg.
The protein related particles and foreign particles remained essentially constantly low over the storage time at the different storage temperatures tested.

### 7.5. Summary of Results

The formulations were stable at all tested pH values over long storage times of up to 24 and 36 months. While temperatures appeared to have an impact on the stability (i.e. higher temperatures inducing more instability related effects), all formulations led to sufficient stability even at high temperatures.

In summary, a pH value of 5.7 and values around 5.7 (e.g. 5.5, 5.9) appeared under the used test conditions to result in an advantageous compromise regarding the storage parameters. For instance, the UP-SEC measurements showed intermediate to low HMW and LMW values for pH 5.7, while the highest and lowest pH each showed highest HMW and LMW contents, respectively. Similar results were obtained in the IEC and HIC measurements.

### 8. Impact of the Acetate Concentration on the Formulation Stability

Formulations containing different concentrations of acetate (see Table 44) were stored over different time points at three different temperatures (5° C., 25° C. and 40° C.).

TABLE 44
Composition of formulations.

| | Acetate/ | | | |
|---|---|---|---|---|
| Formulation | mM | PS20 | Trehalose | pH |
| F1 | 0 | 0.2 mg/mL | 185 mM | 5.7 |
| F2 | 5 | | | |
| F3 | 10 | | | |
| F4 | 15 | | | |
| F5 | 20 | | | |

### 8.1. Preparation of Formulations

The formulations were prepared as described above.

### 8.2. Analytics

Measurements of the samples were performed at 1, 3, 6, 9, 12, 18, 24 and 36 months storage, as well as initially before storage. The storage temperatures were adjusted to 5° C., 25° C. or 40° C. Analytics were performed by measurement using an UP-SEC for monomer, HMW and LMW content and Biacore for the binding activity. Furthermore, the required forces for gliding and break loose force were measured, as well as the osmolality, opalescence and the pH value. Further details on the utilized analysis methods are described below.

### 8.3. Results

### 8.3.1. Measurement of the Monomer Content

UP-SEC analysis was performed to measure the monomer content. Following results were obtained.

TABLE 45
UP-SEC-Monomer-measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 97.9 | 97.9 | 97.9 | 98.0 | 98.0 |
| 5° C. | 3 | 97.6 | 97.6 | 97.7 | 97.7 | 97.7 |
| 5° C. | 6 | 97.5 | 97.5 | 97.6 | 97.6 | 97.5 |
| 5° C. | 9 | 97.2 | — | 97.3 | — | 97.3 |
| 5° C. | 12 | 97.2 | 97.2 | 97.3 | 97.3 | 97.3 |
| 5° C. | 18 | 97.4 | 97.4 | 97.4 | 97.4 | 97.5 |
| 5° C. | 24 | 97.2 | 97.2 | 97.3 | 97.2 | 97.3 |
| 5° C. | 36 | 97.0 | 97.0 | 97.1 | 97.1 | 97.0 |
| 25° C. | 0 | 97.9 | 97.9 | 97.9 | 98.0 | 98.0 |
| 25° C. | 1 | 97.3 | — | 97.3 | — | 97.4 |
| 25° C. | 3 | 96.4 | 96.4 | 96.4 | 96.4 | 96.4 |
| 25° C. | 6 | 95.7 | 95.7 | 95.7 | 95.7 | 95.6 |
| 25° C. | 9 | 94.7 | — | 94.6 | — | 94.6 |
| 25° C. | 12 | 94.1 | 94.1 | 94.1 | 94.1 | 94.1 |
| 40° C. | 0 | 97.9 | 97.9 | 97.9 | 98.0 | 98.0 |
| 40° C. | 1 | 95.2 | — | 95.2 | — | 95.2 |
| 40° C. | 3 | 90.9 | 90.8 | 90.8 | 90.7 | 90.8 |

### Results and Discussion

The monomer measurements show that the formulation is stable over a range of acetate contents, indicating stability for buffer containing and buffer-free formulations with 150 mg/mL risankizumab and formulations according to the present example.

### 8.3.2. Measurement of the HMW Content

Also the HMW content of the formulations was determined via UP-SEC analysis with following results:

TABLE 46
UP-SEC-HMW measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 1.3 | 1.3 | 1.3 | 1.3 | 1.2 |
| 5° C. | 3 | 1.5 | 1.5 | 1.5 | 1.5 | 1.5 |
| 5° C. | 6 | 1.7 | 1.6 | 1.6 | 1.6 | 1.6 |
| 5° C. | 9 | 1.7 | — | 1.6 | — | 1.6 |
| 5° C. | 12 | 1.9 | 1.8 | 1.8 | 1.7 | 1.7 |
| 5° C. | 18 | 1.7 | 1.7 | 1.7 | 1.7 | 1.6 |
| 5° C. | 24 | 2.0 | 2.0 | 2.0 | 2.0 | 2.0 |
| 5° C. | 36 | 2.2 | 2.1 | 2.1 | 2.1 | 2.1 |
| 25° C. | 0 | 1.3 | 1.3 | 1.3 | 1.3 | 1.2 |
| 25° C. | 1 | 1.7 | — | 1.7 | — | 1.7 |
| 25° C. | 3 | 2.3 | 2.2 | 2.2 | 2.2 | 2.2 |
| 25° C. | 6 | 2.5 | 2.5 | 2.4 | 2.5 | 2.5 |
| 25° C. | 9 | 2.7 | — | 2.7 | — | 2.7 |
| 25° C. | 12 | 2.9 | 2.9 | 2.9 | 2.9 | 2.9 |
| 40° C. | 0 | 1.3 | 1.3 | 1.3 | 1.3 | 1.2 |
| 40° C. | 1 | 2.7 | — | 2.6 | — | 2.7 |
| 40° C. | 3 | 4.3 | 4.4 | 4.4 | 4.4 | 4.4 |

### Results and Discussion

The HMW content measurements show that the formulation is stable over a range of acetate contents, indicating stability for buffer containing and buffer-free formulations with 150 mg/mL risankizumab and formulations according to the present example.

### 8.3.3. Measurement of the LMW Content

For LMW content measurements UP-SEC analysis was performed. Following results were obtained.

TABLE 47
UP-SEC-LMW-measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 0.8 | 0.9 | 0.9 | 0.8 | 0.8 |
| 5° C. | 3 | 0.9 | 0.9 | 0.9 | 0.8 | 0.8 |
| 5° C. | 6 | 0.8 | 0.9 | 0.9 | 0.9 | 0.9 |
| 5° C. | 9 | 1.1 | — | 1.1 | — | 1.1 |
| 5° C. | 12 | 1.0 | 1.0 | 1.0 | 1.0 | 1.0 |
| 5° C. | 18 | 0.9 | 0.9 | 0.9 | 0.9 | 0.9 |
| 5° C. | 24 | 0.8 | 0.8 | 0.8 | 0.8 | 0.8 |
| 5° C. | 36 | 0.9 | 0.9 | 0.8 | 0.9 | 0.9 |
| 25° C. | 0 | 0.8 | 0.9 | 0.9 | 0.8 | 0.8 |
| 25° C. | 1 | 1.0 | — | 1.0 | — | 0.9 |
| 25° C. | 3 | 1.4 | 1.4 | 1.4 | 1.4 | 1.4 |
| 25° C. | 6 | 1.8 | 1.8 | 1.9 | 1.8 | 1.9 |
| 25° C. | 9 | 2.7 | — | 2.7 | — | 2.7 |
| 25° C. | 12 | 3.0 | 3.0 | 3.0 | 3.0 | 3.0 |
| 40° C. | 0 | 0.8 | 0.9 | 0.9 | 0.8 | 0.8 |
| 40° C. | 1 | 2.2 | — | 2.2 | — | 2.2 |
| 40° C. | 3 | 4.8 | 4.8 | 4.8 | 4.8 | 4.9 |

### Results and Discussion

The LMW measurements show that the formulation is stable over a range of acetate contents, indicating stability for buffer containing and buffer-free formulations.

### 8.3.4. Measurement of Binding Activity

In order to analyze whether the acetate content has an influence on the binding activity of risankizumab to IL-23, Biacore analysis was performed. The measurements of the binding activity show high binding activity against human IL-23 for all tested formulations and storage times ranging from 92-105% binding activity and 97-100% specific binding activity. These results support the advantageous stability of the tested formulations and indicate that acetate containing and buffer-free formulations are applicable according to the present disclosure.

### 8.3.5. Measurement of Osmolality

As the acetate content also has an influence on the osmolality of the formulation, this parameter was measured. The results are shown in Table 48.

TABLE 48
Osmolality in mOsm/kg of the formulations at
different temperatures and storage times of
formulations comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 288 | 293 | 309 | 322 | 335 |
| 5° C. | 3 | 290 | 294 | 310 | 324 | 335 |
| 5° C. | 6 | 291 | 296 | 311 | 322 | 334 |
| 5° C. | 9 | 304 | — | 309 | — | 333 |
| 5° C. | 12 | 292 | 293 | 305 | 320 | 333 |
| 5° C. | 18 | 284 | 287 | 305 | 322 | 330 |
| 5° C. | 24 | 290 | 290 | 306 | 322 | 333 |
| 5° C. | 36 | 288 | 291 | 306 | 322 | 334 |
| 25° C. | 0 | 288 | 293 | 309 | 322 | 335 |
| 25° C. | 1 | 290 | — | 309 | — | 335 |
| 25° C. | 3 | 288 | 293 | 311 | 322 | 334 |
| 25° C. | 6 | 290 | 295 | 313 | 324 | 334 |
| 25° C. | 9 | 295 | — | 311 | — | 333 |
| 25° C. | 12 | 294 | 294 | 309 | 312 | 333 |
| 40° C. | 0 | 288 | 293 | 309 | 322 | 335 |
| 40° C. | 1 | 287 | — | 308 | — | 334 |
| 40° C. | 3 | 291 | 297 | 314 | 324 | 338 |

### Results and Discussion

The measurements show that the osmolality varies from about 290 to 338 mOsm/kg depending on the amount of added acetate. The more acetate was added, the higher the measured osmolality. An osmolality of approximately 310 mOsm/kg is typically desired and an acetate concentration of 10 mM resulted in the tested formulations in a desired osmolality of around 310 mOsm/kg (measured range from 305 to 314 mOsm/kg). In case another concentration of acetate is required or desired, it may be advantageous to modify the content of the other compounds of the formulation (e.g., another excipient such as trehalose) to adjust the osmolality to approximately 310 mOsm/kg.

### 8.3.6. Measurement of Opalescence

The opalescence of the formulations of the present example was also measured to evaluate the stability. The measured opalescence is overall the same for the different formulations ranging between 4-9 FNU. Higher concentrations of acetate led to a slightly higher opalescence of 7-9 FNU for 20 mM acetate than lower concentrations (4-6 FNU for 0 mM acetate). All formulations according to the present example were stable in view of the measured opalescence.

### 8.3.7. Measurement of pH

In order to determine the pH stability of the formulations comprising varying amounts of buffer, i.e. acetate in this example, the pH values were measured over the time of storage at different temperatures. The results are shown in the subsequent table.

TABLE 49
Measured pH value for the formulations containing
varying amounts of acetate at 5, 25 or 40° C.
over varying storage times.

| | Storage | | | | | |
|---|---|---|---|---|---|---|
| Storage | time, | | | | | |
| condition | months | F1 | F2 | F3 | F4 | F5 |
| 5° C. | 0 | 5.8 | 5.8 | 5.8 | 5.8 | 5.8 |
| 5° C. | 3 | 5.8 | 5.8 | 5.7 | 5.8 | 5.8 |
| 5° C. | 6 | 5.8 | 5.8 | 5.8 | 5.8 | 5.8 |
| 5° C. | 9 | 5.8 | — | 5.8 | — | 5.8 |
| 5° C. | 12 | 5.7 | 5.8 | 5.8 | 5.7 | 5.8 |
| 5° C. | 18 | 5.7 | 5.8 | 5.8 | 5.8 | 5.7 |
| 5° C. | 24 | 5.8 | 5.8 | 5.8 | 5.8 | 5.8 |
| 5° C. | 36 | 5.8 | 5.8 | 5.8 | 5.8 | 5.8 |
| 25° C. | 0 | 5.8 | 5.8 | 5.8 | 5.8 | 5.8 |
| 25° C. | 1 | 5.8 | — | 5.7 | — | 5.7 |
| 25° C. | 3 | 5.8 | 5.7 | 5.8 | 5.8 | 5.7 |
| 25° C. | 6 | 5.8 | 5.8 | 5.8 | 5.8 | 5.8 |
| 25° C. | 9 | 5.8 | — | 5.8 | — | 5.8 |
| 25° C. | 12 | 5.7 | 5.8 | 5.7 | 5.7 | 5.8 |
| 40° C. | 0 | 5.8 | 5.8 | 5.8 | 5.8 | 5.8 |
| 40° C. | 1 | 5.8 | — | 5.8 | — | 5.7 |
| 40° C. | 3 | 5.7 | 5.7 | 5.7 | 5.8 | 5.7 |

### Results and Discussion

The measurements of the pH value demonstrate that the pH is overall kept constant for the tested formulations according to the present example. Therefore, for all acetate contents, including a formulation comprising no acetate, the formulations were stable regarding the pH value.

### 8.3.8. Measurement of Gliding and Break Loose Forces

The maximal and average gliding force were measured as well as the break loose force for syringes containing the different formulations according to the present example. The results of these measurements are shown below.

TABLE 50
Maximum gliding force in N, initially and after the
indicated storage time at 5° C., 25° C. or 40° C. of
formulations comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 6.9 | 6.3 | 6.6 | 7.1 | 6.7 |
| 5° C. | 3 | 7.3 | 7.0 | 6.3 | 7.4 | 7.6 |
| 5° C. | 6 | 7.5 | 6.9 | 6.6 | 7.0 | 6.4 |
| 5° C. | 9 | 8.1 | — | 7.1 | — | 6.6 |
| 5° C. | 12 | 7.5 | 7.0 | 7.1 | 6.7 | 7.1 |
| 5° C. | 18 | 7.9 | 8.8 | 6.1 | 5.9 | 7.0 |
| 5° C. | 24 | 8.8 | 7.7 | 7.1 | 7.4 | 5.9 |
| 5° C. | 36 | 7.8 | 9.3 | 7.0 | 7.4 | 7.1 |
| 25° C. | 0 | 6.9 | 6.3 | 6.6 | 7.1 | 6.7 |
| 25° C. | 1 | 8.1 | — | 5.9 | — | 5.9 |
| 25° C. | 3 | 9.9 | 8.2 | 6.9 | 7.8 | 7.3 |
| 25° C. | 6 | 12.5 | 12.5 | 8.5 | 8.2 | 7.1 |
| 25° C. | 9 | 13.9 | — | 11.8 | — | 10.5 |
| 25° C. | 12 | 18.4 | 14.7 | 14.5 | 13.1 | 10.8 |
| 40° C. | 0 | 6.9 | 6.3 | 6.6 | 7.1 | 6.7 |
| 40° C. | 1 | 11.8 | — | 11.5 | — | 7.6 |
| 40° C. | 3 | 25.1 | 24.0 | 16.4 | 15.6 | 14.2 |

TABLE 51
Average gliding force in N, initially and after the
indicated storage time at 5° C., 25° C. or 40° C. of
formulations comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 6.3 | 6.0 | 6.2 | 6.6 | 6.2 |
| 5° C. | 3 | 6.5 | 6.4 | 6.0 | 6.8 | 6.9 |
| 5° C. | 6 | 7.0 | 6.5 | 6.2 | 6.6 | 6.1 |
| 5° C. | 9 | 7.2 | — | 6.6 | — | 6.2 |
| 5° C. | 12 | 6.9 | 6.5 | 6.6 | 6.3 | 6.7 |
| 5° C. | 18 | 7.2 | 7.9 | 5.8 | 5.7 | 6.6 |
| 5° C. | 24 | 8.2 | 7.2 | 6.6 | 7.0 | 5.6 |
| 5° C. | 36 | 7.0 | 8.1 | 6.6 | 6.8 | 6.7 |
| 25° C. | 0 | 6.3 | 6.0 | 6.2 | 6.6 | 6.2 |
| 25° C. | 1 | 7.3 | — | 5.5 | — | 5.7 |
| 25° C. | 3 | 8.2 | 7.5 | 6.5 | 6.9 | 6.7 |
| 25° C. | 6 | 10.5 | 10.5 | 7.6 | 7.6 | 6.7 |
| 25° C. | 9 | 11.1 | — | 9.5 | — | 9.1 |
| 25° C. | 12 | 14.0 | 11.9 | 11.5 | 10.2 | 8.9 |
| 40° C. | 0 | 6.3 | 6.0 | 6.2 | 6.6 | 6.2 |
| 40° C. | 1 | 9.5 | — | 8.8 | — | 6.8 |
| 40° C. | 3 | 17.2 | 16.2 | 11.9 | 12.0 | 10.4 |

TABLE 52
Break loose force in N, initially and after the indicated storage
time at 5° C., 25° C. or 40° C. of formulations comprising
varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 4.1 | 4.0 | 3.9 | 3.8 | 4.1 |
| 5° C. | 3 | 4.5 | 4.6 | 4.3 | 4.4 | 4.4 |
| 5° C. | 6 | 4.5 | 4.3 | 4.3 | 4.1 | 4.4 |
| 5° C. | 9 | 4.2 | — | 4.5 | — | 4.3 |
| 5° C. | 12 | 4.5 | 4.4 | 4.3 | 4.3 | 4.3 |
| 5° C. | 18 | 4.6 | 4.5 | 4.7 | 4.5 | 4.5 |
| 5° C. | 24 | 4.7 | 4.6 | 4.6 | 4.6 | 4.7 |
| 5° C. | 36 | 4.5 | 4.6 | 4.7 | 4.5 | 4.6 |
| 25° C. | 0 | 4.1 | 4.0 | 3.9 | 3.8 | 4.1 |
| 25° C. | 1 | 4.3 | — | 4.3 | — | 4.7 |
| 25° C. | 3 | 5.0 | 5.4 | 5.0 | 4.8 | 5.2 |
| 25° C. | 6 | 5.1 | 5.4 | 5.1 | 5.0 | 5.2 |
| 25° C. | 9 | 5.2 | — | 5.2 | — | 4.7 |
| 25° C. | 12 | 5.5 | 5.7 | 5.5 | 5.1 | 5.4 |
| 40° C. | 0 | 4.1 | 4.0 | 3.9 | 3.8 | 4.1 |
| 40° C. | 1 | 5.2 | — | 5.0 | — | 5.3 |
| 40° C. | 3 | 6.3 | 6.0 | 6.0 | 5.9 | 6.0 |

### Results and Discussion

The measurements of the gliding force and break loose force show that all formulations are stable and the gliding and break loose forces do not significantly increase over time. Noteworthy, the formulations that do not comprise acetate (F1) or very low concentrations of acetate (F2) show higher forces, indicating that it is useful to add buffer, such as acetate, particularly when aiming at minimizing the required forces applied to a syringe.

### 8.4. Further Analytics and Results

In addition, further analytics were performed for the five formulations tested with the following results. Storage times and temperatures were as described above.
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.8-97.2%, as well as 1.4-1.7% pre peak and 1.5-1.9% post peak. Over 36 months at 5° C. HIC main peak contents in a range of 96.3-97.2% were obtained, as well as 1.4-1.7% pre peak and 1.5-2.2% post peak. At 25° C. for up to 12 months storage time main peak contents between 94.3-97.1% were obtained, as well as 1.4-3.0% pre peak and 1.5-2.7% post peak. At 40° C. for up to 3 months storage time main peak contents between 92.0-97.1% were obtained, as well as 1.4-4.6% pre peak and 1.5-3.4% post peak. No differences between the formulations were observed regarding the main peak, pre peak and post peak.
The protein concentration remained essentially constant up to a storage time of 24 and 36 months at the different storage temperatures tested. Small deviations of protein concentration are due to analytical variations, leading to ranges of 147-155 mg/mL (24 months) and 147-157 mg/mL (36 months).
The dynamic viscosity remained essentially constant up to a storage time of 24 and 36 months at the different storage temperatures tested. The dynamic viscosity was in a range of 8.9-10.0 mPas.
The protein related particles and foreign particles remained essentially constantly low over the storage time at the different storage temperatures tested.

### 8.5. Summary of Results

The tested formulations show overall comparable high stability. Therefore, both acetate containing and buffer-free formulations are suitable for the formulations according to the present disclosure. In view of the required force to be applied to the syringes, solutions containing a buffer such as an acetate buffer have proven to be advantageous over buffer-free formulations. Moreover, in order to achieve an osmolality of 310 mOsm/kg, an acetate content of 10 mM has proven to be suitable considering the other compounds present in the formulation according to the present example.

### IV. Example 3: Analysis of Further Excipients

### 1. Influence of the PS20 Content in Shaking Experiments

Formulations were prepared (see Table 53) wherein the PS20 (polysorbate 20) content was varied from 0, 0.05, 0.075, 0.1, 0.2, 0.3 to 0.5 mg/mL and analyzed during shaking times of 0, 1, 5, 7, 14 and 21 days.

TABLE 53
Composition of formulations.

| | PS20/ | | | |
|---|---|---|---|---|
| Formulation | mg/mL | Acetate | Trehalose | pH |
| F1 | 0.0 | 10 mM | 185 mM | 5.7 |
| F2 | 0.05 | | | |
| F3 | 0.075 | | | |
| F4 | 0.1 | | | |
| F5 | 0.2 | | | |
| F6 | 0.3 | | | |
| F7 | 0.5 | | | |

### 1.1. Preparation of Formulations

The formulations were prepared as described above. The formulations were packaged in 2R vials (1.0 mL) or pre-filled syringes (PFS, Neopak, 1.0 mL) for each formulation, as well as for the control and non-shaken formulations.

### 1.2. Analytics

Measurements of the samples were performed on day 0, 1, 5, 7, 14 and 21. Therefore, the total shaking duration was 21 days for both vial and PFS. The shaking was performed at room temperature (25° C.) at 200 U/min for vials (Orbital shaker) and the movement is adjusted to respective viscosity to ensure air bubble movement for PFS (Tilting shaker (Vari Mix Platform Rocker)). All samples were protected from light. The opalescence of the formulations was measured at the indicated measurement points. Further details on the utilized analysis methods are described below.

### 1.3. Results

### Measurement of the Opalescence

To measure the stability of the formulations containing varying amounts of PS20 and subjected to a shaking stress, the opalescence was measured at different time points. The results obtained by the opalescence measurements are shown below:

TABLE 54
Opalescence in FNU of the formulations having varying amounts
of PS20 in syringes.

| | | Shaking time, days. Storage/shaking condition | | | | | | |
|---|---|---|---|---|---|---|---|---|
| For- | | | 1 | 3 | 7 | 14 | 21 | |
| mu- | | 0 | rock- | rock- | rock- | rock- | rock- | no |
| la- | PS20, | ini- | ing | ing | ing | ing | ing | move- |
| tion | g/l | tial | shaker | shaker | shaker | shaker | shaker | ment |
| F1_S | 0.00 | 8.18 | 8.26 | 8.48 | 11.00 | 15.20 | 19.96 | 7.13 |
| F2_S | 0.05 | 5.72 | 5.82 | 5.70 | 5.78 | 5.86 | 5.83 | 4.97 |
| F3_S | 0.08 | 5.90 | 5.95 | 5.68 | 6.53 | 6.13 | 5.67 | 5.49 |
| F4_S | 0.10 | 5.61 | 5.19 | 5.94 | 6.12 | 6.10 | 6.01 | 5.47 |
| F5_S | 0.20 | 6.32 | 5.93 | 5.60 | 6.12 | 6.33 | 6.30 | 5.12 |
| F6_S | 0.30 | 6.22 | 6.23 | 5.75 | 5.55 | 5.75 | 5.87 | 5.06 |
| F7_S | 0.50 | 6.10 | 5.77 | 5.61 | 6.01 | 7.33 | 5.78 | 5.32 |

TABLE 55
Opalescence in FNU of the formulations having varying amounts
of PS20 in vials.

| | | Shaking time, days. Storage/shaking conditon | | | | | | |
|---|---|---|---|---|---|---|---|---|
| | | | 1 | 3 | 7 | 14 | 21 | |
| For- | | 0 | hori- | hori- | hori- | hori- | hori- | no |
| mula- | PS20, | ini- | zontal | zontal | zontal | zontal | zontal | move- |
| tion | g/l | tial | shaker | shaker | shaker | shaker | shaker | ment |
| F1_V | 0.00 | 6.83 | 8.29 | 8.37 | 10.72 | 14.52 | 22.50 | 7.96 |
| F2_V | 0.05 | 6.70 | 5.70 | 6.10 | 5.45 | 5.91 | 5.72 | 5.46 |
| F3_V | 0.08 | 5.84 | 5.47 | 6.57 | 5.85 | 6.26 | 6.86 | 6.21 |
| F4_V | 0.10 | 5.67 | 5.10 | 6.24 | 5.39 | 5.75 | 6.28 | 5.81 |
| F5_V | 0.20 | 7.23 | 6.21 | 5.94 | 6.07 | 5.58 | 6.87 | 6.89 |
| F6_V | 0.30 | 6.60 | 5.20 | 5.89 | 5.36 | 5.78 | 5.61 | 5.66 |
| F7_V | 0.50 | 5.70 | 5.62 | 5.61 | 5.81 | 5.54 | 5.48 | 5.89 |

### 1.4. Summary of Results

The shaking study clearly revealed that formulations that do not comprise PS20 significantly increased in opalescence over the shaking time of 21 days. In contrast, all formulations that comprised PS20, i.e. even the lowest amount of 0.05 g/L, showed no increase in opalescence over time. The results substantiate the importance of a surfactant such as the non-ionic surfactant PS20 in formulations according to the present disclosure, in particular formulations comprising 150 mg/mL risankizumab.

### 2. Influence of the PS20 Content During Storage

The prepared formulations (see Table 53) were analyzed over different time points stored at three different temperatures (5° C., 25° C. and 40° C.).

### 2.1. Analytics

Measurements of the samples were performed at 1, 3, 6, 9, 12, 18, 24 and 36 months storage, as well as initially before storage. UP-SEC analysis was performed in order to determine the monomer, HM/W and LMW contents. Moreover, sub-visible particle content, gliding force and break loose force were measured. Further details on the utilized analysis methods are described below.

### 2.2. Results

### 2.2.1. Measurement of the Monomer Content

The stability of the formulations was assessed by measuring the monomer content using the UP-SEC analysis. The results are shown below.

TABLE 56
UP-SEC-Monomer-measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 97.9 | 97.9 | 97.9 | 97.9 | 97.8 | 97.8 | 97.8 |
| 5° C. | 3 | 97.5 | 97.5 | 97.6 | 97.5 | 97.5 | 97.5 | 97.5 |
| 5° C. | 6 | 97.3 | 97.3 | 97.3 | 97.3 | 97.3 | 97.3 | 97.2 |
| 5° C. | 9 | 97.1 | 97.1 | 97.1 | 97.1 | 97.1 | 97.1 | 97.1 |
| 5° C. | 12 | 97.2 | 97.2 | 97.2 | 97.2 | 97.2 | 97.2 | 97.2 |
| 5° C. | 18 | 97.4 | 97.3 | 97.3 | 97.5 | 97.4 | 97.4 | 97.4 |
| 5° C. | 24 | 97.1 | 97.2 | 97.2 | 97.1 | 97.2 | 97.1 | 97.2 |
| 5° C. | 36 | 96.8 | 96.9 | 96.9 | 96.9 | 97.0 | 97.0 | 97.0 |
| 25° C. | 1 | 97.2 | 97.2 | 97.2 | 97.2 | 97.2 | 97.2 | 97.2 |
| 25° C. | 3 | 96.2 | 96.2 | 96.3 | 96.2 | 96.2 | 96.3 | 96.2 |
| 25° C. | 6 | 95.2 | 95.3 | 95.4 | 95.4 | 95.4 | 95.4 | 95.4 |
| 25° C. | 9 | 94.2 | 94.4 | 94.5 | 94.5 | 94.5 | 94.5 | 94.5 |
| 25° C. | 12 | 93.5 | 93.8 | 93.9 | 93.9 | 94.0 | 94.0 | 93.9 |
| 40° C. | 1 | 94.9 | 94.9 | 94.9 | 94.9 | 94.9 | 94.8 | 94.8 |
| 40° C. | 3 | 89.6 | 90.1 | 90.4 | 90.3 | 90.4 | 90.4 | 89.7 |

### Results and Discussion

The monomer measurements show that the formulation is stable over a range of PS20 contents. Particular high monomer values were obtained for PS20 contents around 0.2 mg/mL.

### 2.2.2. Measurement of the HMW Content

The stability of the formulations was further assessed by measuring the HMW content, again using UP-SEC. The results are shown below.

TABLE 57
UP-SEC HMW measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 1.3 | 1.4 | 1.4 | 1.4 | 1.4 | 1.4 | 1.4 |
| 5° C. | 3 | 1.7 | 1.7 | 1.7 | 1.7 | 1.7 | 1.7 | 1.7 |
| 5° C. | 6 | 1.9 | 1.9 | 1.9 | 1.9 | 1.9 | 1.9 | 1.9 |
| 5° C. | 9 | 1.8 | 1.8 | 1.8 | 1.8 | 1.8 | 1.8 | 1.8 |
| 5° C. | 12 | 1.0 | 1.0 | 1.0 | 1.0 | 1.0 | 1.0 | 1.0 |
| 5° C. | 18 | 1.7 | 1.8 | 1.8 | 1.7 | 1.7 | 1.7 | 1.7 |
| 5° C. | 24 | 2.1 | 2.1 | 2.0 | 2.1 | 2.1 | 2.1 | 2.1 |
| 5° C. | 36 | 2.3 | 2.2 | 2.2 | 2.2 | 2.2 | 2.2 | 2.2 |
| 25° C. | 1 | 1.9 | 1.9 | 1.9 | 1.9 | 1.9 | 1.9 | 1.9 |
| 25° C. | 3 | 2.4 | 2.4 | 2.4 | 2.4 | 2.4 | 2.4 | 2.5 |
| 25° C. | 6 | 2.9 | 2.8 | 2.8 | 2.8 | 2.8 | 2.8 | 2.8 |
| 25° C. | 9 | 3.0 | 2.9 | 2.8 | 2.8 | 2.8 | 2.8 | 2.8 |
| 25° C. | 12 | 3.2 | 3.1 | 3.1 | 3.1 | 3.0 | 3.0 | 3.1 |
| 40° C. | 1 | 2.9 | 2.9 | 2.9 | 2.9 | 2.9 | 2.9 | 3.0 |
| 40° C. | 3 | 5.1 | 4.9 | 4.7 | 4.7 | 4.7 | 4.7 | 5.3 |

### Results and Discussion

The HMW content correlates with the monomer measurements. Overall, the tested formulations were stable over a range of PS20 contents. Particular low increases of the HMW contents were obtained for PS20 contents of 0.2 mg/mL. However, highest and lowest tested PS20 contents appear to have led to slightly higher HMW values.

### 2.2.3. Measurement of the LMW Content

Also the LMW contents were measured using an UP-SEC and following results were obtained:

TABLE 58
UP-SEC-LMW-measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 0.8 | 0.8 | 0.8 | 0.8 | 0.8 | 0.8 | 0.8 |
| 5° C. | 3 | 0.8 | 0.8 | 0.8 | 0.8 | 0.8 | 0.8 | 0.8 |
| 5° C. | 6 | 0.8 | 0.8 | 0.8 | 0.8 | 0.8 | 0.8 | 0.8 |
| 5° C. | 9 | 1.1 | 1.1 | 1.1 | 1.1 | 1.1 | 1.1 | 1.1 |
| 5° C. | 12 | 1.9 | 1.8 | 1.8 | 1.8 | 1.8 | 1.8 | 1.8 |
| 5° C. | 18 | 0.8 | 1.0 | 1.0 | 0.8 | 0.9 | 0.9 | 0.9 |
| 5° C. | 24 | 0.8 | 0.8 | 0.8 | 0.8 | 0.8 | 0.8 | 0.8 |
| 5° C. | 36 | 0.9 | 0.9 | 0.9 | 0.9 | 0.9 | 0.9 | 0.9 |
| 25° C. | 1 | 1.0 | 1.0 | 1.0 | 1.0 | 1.0 | 1.0 | 0.9 |
| 25° C. | 3 | 1.4 | 1.3 | 1.3 | 1.4 | 1.4 | 1.3 | 1.3 |
| 25° C. | 6 | 1.9 | 1.9 | 1.8 | 1.8 | 1.8 | 1.8 | 1.8 |
| 25° C. | 9 | 2.8 | 2.7 | 2.7 | 2.7 | 2.7 | 2.7 | 2.7 |
| 25° C. | 12 | 3.3 | 3.1 | 3.1 | 3.1 | 3.0 | 3.0 | 3.0 |
| 40° C. | 1 | 2.2 | 2.3 | 2.2 | 2.2 | 2.2 | 2.3 | 2.3 |
| 40° C. | 3 | 5.3 | 5.1 | 5.0 | 5.0 | 4.9 | 4.9 | 5.0 |

### Results and Discussion

The LMW content correlates with the monomer measurements. Overall, the tested formulations were stable over a range of PS20 contents. Particular low increases of the LMW contents were obtained for PS 20 contents of 0.2 mg/mL. The lowest tested PS20 content (see F1) appears to have led to slightly higher LMW values.

### 2.2.4. Measurement of the Opalescence

Moreover, the opalescence was measured for the formulations containing the varying amounts of PS20. The results are depicted below.

TABLE 59
Opalescence measurements in FNU of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 8 | 6 | 6 | 6 | 6 | 6 | 6 |
| 5° C. | 3 | 7 | 6 | 6 | 6 | 7 | 6 | 6 |
| 5° C. | 6 | 6 | 6 | 6 | 6 | 7 | 6 | 6 |
| 5° C. | 9 | 12 | 5 | 7 | 6 | 6 | 7 | 8 |
| 5° C. | 12 | 11 | 10 | 8 | 7 | 7 | 6 | 7 |
| 5° C. | 18 | 19 | 9 | 12 | 8 | 7 | 7 | 6 |
| 5° C. | 24 | 13 | 6 | 7 | 7 | 7 | 7 | 7 |
| 5° C. | 36 | 12 | 6 | 7 | 7 | 7 | 7 | 7 |
| 25° C. | 1 | 6 | 6 | 5 | 6 | 5 | 6 | 6 |
| 25° C. | 3 | 6 | 6 | 6 | 6 | 6 | 6 | 6 |
| 25° C. | 6 | 6 | 6 | 6 | 6 | 7 | 7 | 7 |
| 25° C. | 9 | 6 | 6 | 6 | 7 | 6 | 7 | 8 |
| 25° C. | 12 | 7 | 7 | 7 | 7 | 8 | 7 | 9 |
| 40° C. | 1 | 7 | 6 | 6 | 6 | 6 | 7 | 6 |
| 40° C. | 3 | 7 | 6 | 6 | 7 | 7 | 7 | 8 |

### Results and Discussion

The measurements show that at higher temperatures of 25 and 40° C. all formulations led to no increase in opalescence. However, at a temperature of 5° C. and later storage time points (e.g. 18, 24 and 36 months), formulation F1 (no PS20) showed an increase in opalescence. Hence, incorporating a surfactant such as the non-ionic surfactant PS20 is advantageous.

### 2.2.5. Measurement of the Sub-Visible Particle Content

The formulations were analyzed in regard to their sub-visible particle content (≥2 μm, ≥10 μm and >25 μm) over 24 and 36 months stored at 5FC.

TABLE 60
Measurement of the sub-visible particle content of particles with
a size of ≥2, ≥10 and ≥25 μm stored for up to 24 and 36 months
at 5° C. of formulations comprising varying amounts of PS20.

| Treat- | Particle | Number of measured particles | | | | | | |
|---|---|---|---|---|---|---|---|---|
| ment | size | F1 | F2 | F3 | F4 | F5 | F6 | F7 |
| Initial | ≥2 μm | 58318 | 1876 | 4832 | 3607 | 7780 | 5121 | 6578 |
| | ≥10 μm | 6061 | 70 | 99 | 88 | 312 | 111 | 278 |
| | ≥25 μm | 183 | 9 | 5 | 2 | 31 | 9 | 26 |
| 5° C., 3 | ≥2 μm | 41237 | 1442 | 4559 | 9475 | 14173 | 10810 | 4201 |
| months | ≥10 μm | 3650 | 48 | 170 | 225 | 177 | 224 | 94 |
| | ≥25 μm | 196 | 2 | 6 | 15 | 1 | 3 | 1 |
| 5° C., 6 | ≥2 μm | 51615 | 4949 | 3580 | 5282 | 11069 | 16838 | 18125 |
| months | ≥10 μm | 3501 | 38 | 48 | 45 | 71 | 105 | 131 |
| | ≥25 μm | 29 | 0 | 2 | 0 | 1 | 6 | 1 |
| 5° C., 9 | ≥2 μm | 70311 | 11911 | 6453 | 6982 | 35316 | 6358 | 10660 |
| months | ≥10 μm | 5195 | 42 | 17 | 60 | 60 | 33 | 79 |
| | ≥25 μm | 122 | 1 | 0 | 2 | 0 | 2 | 9 |
| 5° C., 12 | ≥2 μm | 58322 | 16911 | 9064 | 28517 | 7761 | 15372 | 41782 |
| months | ≥10 μm | 4529 | 121 | 183 | 360 | 141 | 132 | 295 |
| | ≥25 μm | 184 | 2 | 7 | 7 | 1 | 0 | 6 |
| 5° C., 18 | ≥2 μm | 28849 | 4496 | 5760 | 9250 | 7025 | 18772 | 20556 |
| months | ≥10 μm | 2939 | 79 | 84 | 123 | 56 | 181 | 232 |
| | ≥25 μm | 181 | 2 | 6 | 1 | 3 | 2 | 6 |
| 5° C., 24 | ≥2 μm | 61469 | 5862 | 9427 | 7969 | 17688 | 37040 | 3688 |
| months | ≥10 μm | 5407 | 84 | 163 | 197 | 355 | 290 | 148 |
| | ≥25 μm | 66 | 1 | 9 | 9 | 6 | 3 | 5 |
| 5° C., 36 | ≥2 μm | 17157 | 4306 | 7984 | 12335 | 16377 | 21911 | 14799 |
| months | ≥10 μm | 2255 | 49 | 54 | 146 | 172 | 136 | 65 |
| | ≥25 μm | 257 | 1 | 2 | 8 | 5 | 2 | 3 |

### Results and Discussion

The measurements of the sub-visible particle content show that all formulations are stable at SoC for up to 24 and 36 months. Only formulations without PS20 (F1) appeared to result in some particle formation, substantiating that it is advantageous to add a surfactant such as the non-ionic surfactant PS20 to the formulations according to the present disclosure.

### 2.2.6. Measurement of the Gliding Force and Break Loose Force

The maximal and average gliding force as well as the break loose force were measured for the formulations comprising varying amounts of PS20. The results of the measurements are shown below.

TABLE 61
Maximal gliding force in N. The formulations comprised
varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 7.2 | 7.1 | 6.2 | 6.5 | 6.4 | 6.3 | 6.9 |
| 5° C. | 3 | 7.8 | 7.4 | 8.1 | 6.8 | 6.8 | 6.5 | 6.4 |
| 5° C. | 6 | 7.7 | 7.0 | 6.7 | 6.6 | 6.1 | 6.5 | 6.7 |
| 5° C. | 9 | 8.7 | 6.4 | 7.4 | 7.6 | 6.7 | 6.6 | 8.2 |
| 5° C. | 12 | 7.1 | 7.5 | 6.7 | 7.2 | 5.9 | 6.7 | 7.3 |
| 5° C. | 18 | 8.9 | 7.4 | 6.1 | 6.9 | 6.1 | 7.4 | 7.1 |
| 5° C. | 24 | 8.1 | 6.2 | 7.6 | 8.0 | 7.2 | 6.7 | 7.1 |
| 5° C. | 36 | 9.2 | 8.5 | 9.1 | 7.0 | 7.5 | 6.7 | 7.2 |
| 25° C. | 1 | 7.4 | 6.5 | 6.4 | 6.8 | 6.6 | 6.1 | 6.2 |
| 25° C. | 3 | 8.1 | 7.0 | 7.4 | 7.7 | 7.0 | 7.7 | 7.7 |
| 25° C. | 6 | 8.1 | 7.3 | 9.4 | 8.9 | 9.9 | 9.6 | 10.8 |
| 25° C. | 9 | 7.2 | 11.9 | 10.2 | 9.8 | 11.2 | 11.3 | 12.6 |
| 25° C. | 12 | 8.4 | 11.1 | 12.0 | 13.6 | 12.8 | 15.1 | 16.1 |
| 40° C. | 1 | 7.7 | 8.1 | 9.2 | 11.2 | 8.7 | 9.5 | 8.6 |

TABLE 62
Average gliding force in N. The formulations comprised
varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 6.5 | 6.5 | 5.8 | 6.1 | 5.9 | 5.9 | 6.4 |
| 5° C. | 3 | 7.1 | 6.7 | 7.3 | 6.3 | 6.2 | 6.0 | 6.0 |
| 5° C. | 6 | 6.8 | 6.5 | 6.3 | 6.2 | 5.7 | 6.1 | 6.2 |
| 5° C. | 9 | 7.5 | 6.1 | 6.7 | 6.9 | 6.3 | 6.1 | 7.2 |
| 5° C. | 12 | 6.4 | 6.8 | 6.4 | 6.8 | 5.7 | 6.2 | 6.7 |
| 5° C. | 18 | 7.9 | 7.0 | 5.8 | 6.4 | 5.8 | 6.8 | 6.6 |
| 5° C. | 24 | 7.2 | 5.9 | 7.0 | 7.3 | 6.7 | 6.4 | 6.6 |
| 5° C. | 36 | 7.8 | 7.7 | 8.2 | 6.6 | 6.8 | 6.4 | 6.6 |
| 25° C. | 1 | 6.7 | 6.0 | 6.0 | 6.4 | 6.2 | 5.8 | 5.8 |
| 25° C. | 3 | 7.4 | 6.5 | 6.9 | 7.2 | 6.3 | 7.0 | 7.2 |
| 25° C. | 6 | 7.3 | 6.9 | 8.4 | 7.8 | 8.0 | 8.3 | 9.2 |
| 25° C. | 9 | 6.4 | 10.0 | 8.5 | 8.6 | 9.1 | 9.5 | 9.9 |
| 25° C. | 12 | 7.7 | 9.6 | 10.0 | 11.2 | 10.5 | 12.1 | 12.4 |
| 40° C. | 1 | 6.9 | 7.0 | 7.9 | 9.2 | 7.0 | 8.0 | 7.5 |

TABLE 63
Break loose force in N. The formulations comprised
varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 4.1 | 4.3 | 4.2 | 4.1 | 4.2 | 4.3 | 4.1 |
| 5° C. | 3 | 4.7 | 4.6 | 4.8 | 4.8 | 4.6 | 4.7 | 4.7 |
| 5° C. | 6 | 4.6 | 4.3 | 4.5 | 4.5 | 4.5 | 4.5 | 4.7 |
| 5° C. | 9 | 3.8 | 4.1 | 4.0 | 4.1 | 3.9 | 4.2 | 4.2 |
| 5° C. | 12 | 4.5 | 4.5 | 4.5 | 4.7 | 4.7 | 4.3 | 4.5 |
| 5° C. | 18 | 4.6 | 4.9 | 4.8 | 4.6 | 4.7 | 4.6 | 4.7 |
| 5° C. | 24 | 5.0 | 5.1 | 4.8 | 4.7 | 4.8 | 4.6 | 4.8 |
| 5° C. | 36 | 4.2 | 4.3 | 4.3 | 4.0 | 4.0 | 4.5 | 4.6 |
| 25° C. | 1 | 4.8 | 4.8 | 4.6 | 4.7 | 4.7 | 4.4 | 4.7 |
| 25° C. | 3 | 5.1 | 5.4 | 5.3 | 5.5 | 5.4 | 5.2 | 5.4 |
| 25° C. | 6 | 5.1 | 5.1 | 5.1 | 5.3 | 4.9 | 5.1 | 5.3 |
| 25° C. | 9 | 4.4 | 4.9 | 4.4 | 5.0 | 5.0 | 4.9 | 5.0 |
| 25° C. | 12 | 5.4 | 5.4 | 5.4 | 5.7 | 5.3 | 5.6 | 5.3 |
| 40° C. | 1 | 5.0 | 5.0 | 5.0 | 5.2 | 5.1 | 4.9 | 5.0 |
| 40° C. | 3 | 5.9 | 5.8 | 6.1 | 6.3 | 6.0 | 6.0 | 6.4 |

### Results and Discussion

The measurements of the gliding force revealed relatively high gliding forces for high contents of PS20 of 0.5 g/L in comparison to the lower contents. While a content of 0 g/L showed lowest increase in gliding force, an intermediate concentration of 0.2 g/L showed a good compromise between high and low gliding force. Noteworthy, essentially no differences between the formulations in respect to the break loose force were observed.

### 2.3. Further Analytics and Results

In addition, further analytics were performed for the seven formulations tested with the following results. Storage times and temperatures were as described above.
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.5-97.3%, as well as 1.4-1.7% pre peak and 1.4-1.9% post peak. Over 36 months at 5° C. HIC main peak contents in a range of 95.9-97.3% were obtained, as well as 1.4-1.7% pre peak and 1.4-2.4% post peak. At 25° C. for up to 12 months storage time main peak contents between 93.9-96.8% were obtained, as well as 1.4-3.0% pre peak and 1.7-2.7% post peak. At 40° C. for up to 3 months storage time main peak contents between 90.3-95.2% were obtained, as well as 2.5-6.0% pre peak and 2.3-3.7% post peak. No differences between the formulations were observed regarding the main peak, pre peak and post peak.
The specific binding activity remained essentially constant up to a storage time of 24 and 36 months at the different storage temperatures tested. The specific binding activity was in a range of 97-101%.
The protein concentration remained essentially constant up to a storage time of 24 and 36 months at the different storage temperatures tested. Small deviations of protein concentration are due to analytical variations, leading to ranges of 147-155 mg/mL (24 months) and 147-159 mg/mL (36 months).
The pH value remained essentially constant up to a storage time of 24 and 36 months at the different storage temperatures tested. The pH was in a range of 5.7-5.9.
The osmolality remained essentially constant up to a storage time of 24 and 36 months at the different storage temperatures tested. The tested values ranged from 305-322 mOsm/kg.
The dynamic viscosity remained essentially constant up to a storage time of 24 and 36 months at the different storage temperatures tested. The dynamic viscosity was in a range of 9.2-11.0 mPas.
The protein related particles and foreign particles remained essentially constantly low over the storage time at the different storage temperatures tested.

### 2.4. Summary of Results

In summary, the tested formulations were stable over long term storage up to 24 and 36 months at temperatures ranging from 5° C. up to 40° C. In particular, formulations comprising a surfactant such as PS20 were found to be stable, whereas formulation lacking PS20 showed some formation of sub-visible particles and an increase in opalescence. Also the LMW content was slightly increased for formulations lacking PS20. A particularly suitable content of a surfactant such as the non-ionic surfactant PS20 appeared to be 0.2 g/L under the tested conditions.

3. Variation of the trehalose content

In this example the trehalose concentration was varied from 145, 165, 185, 205 to 225 mM and analyzed over different time points stored at three different temperatures (5° C., 25° C. and 40° C.). The prepared formulations are shown in Table 64.

TABLE 64
Composition of formulations.

| | Trehalose/ | PS20/ | | |
|---|---|---|---|---|
| Formulation | mM | mg/mL | Acetate/mM | pH |
| F1 | 145 | 0.2 | 10 | 5.7 |
| F2 | 165 | | | |
| F3 | 185 | | | |
| F4 | 205 | | | |
| F5 | 225 | | | |

### 3.1. Analytics

Measurements of the samples were performed at 1, 3, 6, 9, 12, 18, 24 and 36 months storage, as well as initially before storage. Further details on the utilized analysis methods are described below.

### 3.2. Results

### 3.2.1. Measurement of the Monomer Content

The stability of the formulations comprising varying trehalose amounts was assessed by measuring the monomer content using an UP-SEC analysis, revealing the results shown below.

TABLE 65
UP-SEC-Monomer-measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 98.1 | 98.0 | 98.0 | 98.1 | 98.0 |
| 5° C. | 3 | 97.7 | 97.7 | 97.7 | 97.7 | 97.6 |
| 5° C. | 6 | 97.6 | 97.5 | 97.5 | 97.5 | 97.5 |
| 5° C. | 9 | 97.2 | — | 97.2 | — | 97.1 |
| 5° C. | 12 | 97.2 | 97.3 | 97.2 | 97.3 | 97.2 |
| 5° C. | 18 | 97.5 | 97.5 | 97.4 | 97.4 | 97.3 |
| 5° C. | 24 | 97.2 | 97.1 | 97.2 | 97.2 | 97.1 |
| 5° C. | 36 | 97.0 | 97.0 | 97.0 | 97.0 | 96.9 |
| 25° C. | 0 | 98.1 | 98.0 | 98.0 | 98.1 | 98.0 |
| 25° C. | 1 | 97.2 | — | 97.3 | — | 97.2 |
| 25° C. | 3 | 96.5 | 96.5 | 96.4 | 96.4 | 96.3 |
| 25° C. | 6 | 95.7 | 95.6 | 95.6 | 95.6 | 95.6 |
| 25° C. | 9 | 94.6 | — | 94.6 | — | 94.6 |
| 25° C. | 12 | 94.1 | 94.1 | 94.1 | 94.1 | 94.1 |
| 40° C. | 0 | 98.1 | 98.0 | 98.0 | 98.1 | 98.0 |
| 40° C. | 1 | 95.0 | — | 95.0 | — | 95.0 |
| 40° C. | 3 | 90.7 | 90.8 | 90.8 | 90.7 | 90.8 |

### Results and Discussion

The monomer measurements show that the formulation is stable over a range of trehalose contents, indicating stability over a range of trehalose contents.

### 3.2.2. Measurement of the HMW Content

The HMW contents of the formulations were measured using UP-SEC. The results of the analysis are shown below.

TABLE 66
UP-SEC HMW measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 1.1 | 1.1 | 1.1 | 1.1 | 1.2 |
| 5° C. | 3 | 1.4 | 1.4 | 1.5 | 1.4 | 1.5 |
| 5° C. | 6 | 1.5 | 1.6 | 1.6 | 1.6 | 1.6 |
| 5° C. | 9 | 1.7 | — | 1.7 | — | 1.8 |
| 5° C. | 12 | 1.8 | 1.8 | 1.8 | 1.8 | 1.9 |
| 5° C. | 18 | 1.7 | 1.7 | 1.7 | 1.7 | 1.8 |
| 5° C. | 24 | 2.0 | 2.0 | 2.0 | 2.0 | 2.1 |
| 5° C. | 36 | 2.1 | 2.1 | 2.2 | 2.2 | 2.2 |
| 25° C. | 0 | 1.1 | 1.1 | 1.1 | 1.1 | 1.2 |
| 25° C. | 1 | 1.7 | — | 1.7 | — | 1.7 |
| 25° C. | 3 | 2.2 | 2.2 | 2.2 | 2.2 | 2.3 |
| 25° C. | 6 | 2.4 | 2.4 | 2.5 | 2.4 | 2.5 |
| 25° C. | 9 | 2.7 | — | 2.7 | — | 2.8 |
| 25° C. | 12 | 2.9 | 3.0 | 3.0 | 3.0 | 3.0 |
| 40° C. | 0 | 1.1 | 1.1 | 1.1 | 1.1 | 1.2 |
| 40° C. | 1 | 2.8 | — | 2.7 | — | 2.8 |
| 40° C. | 3 | 4.4 | 4.3 | 4.3 | 4.3 | 4.4 |

### Results and Discussion

The HMW content measurements show that the formulation is stable over a range of trehalose contents.

### 3.2.3. Measurement of the LMW Content

Also the LMW content was measured via UP-SEC for the formulations comprising varying amounts of trehalose. The results are shown below.

TABLE 67
UP-SEC-LMW-measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 0.9 | 0.9 | 0.9 | 0.9 | 0.9 |
| 5° C. | 3 | 0.9 | 0.9 | 0.9 | 0.9 | 0.9 |
| 5° C. | 6 | 0.9 | 0.9 | 0.9 | 0.9 | 0.9 |
| 5° C. | 9 | 1.1 | — | 1.1 | — | 1.1 |
| 5° C. | 12 | 1.0 | 1.0 | 1.0 | 1.0 | 1.0 |
| 5° C. | 18 | 0.9 | 0.9 | 0.9 | 0.9 | 0.9 |
| 5° C. | 24 | 0.8 | 0.8 | 0.8 | 0.8 | 0.8 |
| 5° C. | 36 | 0.9 | 0.8 | 0.9 | 0.9 | 0.9 |
| 25° C. | 0 | 0.9 | 0.9 | 0.9 | 0.9 | 0.9 |
| 25° C. | 1 | 1.1 | — | 1.0 | — | 1.0 |
| 25° C. | 3 | 1.4 | 1.4 | 1.4 | 1.4 | 1.4 |
| 25° C. | 6 | 1.9 | 1.9 | 1.9 | 1.9 | 1.9 |
| 25° C. | 9 | 2.6 | — | 2.6 | — | 2.6 |
| 25° C. | 12 | 2.9 | 2.9 | 2.9 | 3.0 | 2.9 |
| 40° C. | 0 | 0.9 | 0.9 | 0.9 | 0.9 | 0.9 |
| 40° C. | 1 | 2.3 | — | 2.3 | — | 2.3 |
| 40° C. | 3 | 4.9 | 4.9 | 4.9 | 4.9 | 4.9 |

### Results and Discussion

The LMW measurements show that the formulation is stable over a range of trehalose contents.

### 3.2.4. Measurement of Binding Activity

The binding activity of the risankizumab comprised in the formulations according to the present disclosure was measured. The measurements of antigen binding show high binding activity to IL-23 for all tested formulations ranging from 92-122% binding activity and 96-10000 specific binding activity. These results support the advantageous stability of the tested formulations and indicate that trehalose containing formulations at various concentrations are applicable according to the present disclosure.

### 3.2.5. Measurement of the Osmolality

The osmolality was measured in order to assure that the tested formulations have a suitable osmolality for injection. The results are shown below:

TABLE 68
Measured osmolality in mOsm/kg of the formulations
comprising varying amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 246 | 274 | 309 | 337 | 376 |
| 5° C. | 3 | 247 | 277 | 307 | 340 | 378 |
| 5° C. | 6 | 247 | 275 | 309 | 338 | 375 |
| 5° C. | 9 | 251 | — | 310 | — | 380 |
| 5° C. | 12 | 245 | 274 | 305 | 335 | 375 |
| 5° C. | 18 | 248 | 271 | 303 | 340 | 370 |
| 5° C. | 24 | 248 | 277 | 308 | 339 | 376 |
| 5° C. | 36 | 248 | 275 | 307 | 338 | 374 |
| 25° C. | 0 | 246 | 274 | 309 | 337 | 376 |
| 25° C. | 1 | 248 | — | 307 | — | 373 |
| 25° C. | 3 | 252 | 287 | 311 | 338 | 378 |
| 25° C. | 6 | 246 | 276 | 310 | 335 | 371 |
| 25° C. | 9 | 253 | — | 310 | — | 380 |
| 25° C. | 12 | 241 | 278 | 302 | 336 | 377 |
| 40° C. | 0 | 246 | 274 | 309 | 337 | 376 |
| 40° C. | 1 | 245 | | 310 | | 373 |
| 40° C. | 3 | 249 | 278 | 312 | 340 | 379 |

### Results and Discussion

The osmolality values range from around 245 to 380 mOsm/kg for trehalose concentrations from 145 to 225 mM. As an optimal osmolality is around 310 mOsm/kg, it can be advantageous to provide formulations having such osmolality. This can for instance be achieved using a trehalose concentration of 185 mM in combination with the formulation according to the present example.

### 3.3. Further Analytics and Results

In addition, further analytics were performed for the five formulations tested (storage times and temperatures were as described above).
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.4-97.4%, as well as 1.4-1.8% pre peak and 1.2-2.0% post peak. Over 36 months at 5° C. HIC main peak contents in a range of 96.0-97.4% were obtained, as well as 1.4-1.8% pre peak and 1.2-2.3% post peak. At 25° C. for up to 12 months storage time main peak contents between 94.2-97.4% were obtained, as well as 1.4-3.0% pre peak and 1.2-2.8% post peak. At 40° C. for up to 3 months storage time main peak contents between 9.3-97.4% were obtained, as well as 1.4-5.9% pre peak and 1.2-3.7% post peak. No differences between the formulations were observed regarding the main peak, pre peak and post peak.
The protein concentration remained essentially constant up to a storage time of 24 and 36 months at the different storage temperatures tested. Small deviations of protein concentration are due to analytical variations, leading to ranges of 145-153 mg/mL (24 months) and 148-158 mg/mL (36 months).
The pH value remained essentially constant up to a storage time of 24 and 36 months at the different storage temperatures tested. The pH was in a range of 5.7-5.9.
The opalescence remained essentially constant up to a storage time of 24 and 36 months at the different storage temperatures tested. The opalescence was in a range of 5-9 FNU.
The dynamic viscosity remained essentially constant up to a storage time of 24 and 36 months at the different storage temperatures tested. The dynamic viscosity was in a range of 8.9-10.3 mPas.
The gliding forces remained constant over 24 months at 5° C. in a range of 6.5-7.7 N (maximum) and 5.8-7.4 N (average), as well 3.9-5.0 N for break loose force. Over 36 months at 5° C. gliding forces remained constant in a range of 6.1-8.5 N (maximum) and 5.8-7.7 N (average), as well 3.9-5.0 N for break loose force. At 25° C. for up to 12 months storage time gliding forces range between 6.7-15.7 N (maximum) and 6.2-12.4 N (average), as well as 3.9-5.6 N for the break loose force. At 40° C. for up to 3 months storage time gliding forces range between 8.7-23.1 N (maximum) and 7.3-16.4 N (average), as well as 5.1-6.6 N for the break loose force.
The protein related particles and foreign particles remained essentially constantly low over the storage time at the different storage temperatures tested.

### 3.4. Summary of the Results

In summary, all tested formulations were stable, substantiating that the trehalose concentration can be varied while maintaining a high stability. Therefore, the indicated trehalose concentrations can be flexibly applied in order to produce stable protein formulations of 150 mg/mL risankizumab.

### V. Example 4: Analysis of Further Parameters of a Specific Formulation

In view of the results of the prior examples, a particularly suitable formulation comprises the following compounds:
150 mg/mL risankizumab,
10 mM acetate buffer,
185 mM trehalose, and
0.2 mg/mL PS20;
   wherein the formulation has a pH of 5.7.

The appearance of this formulation was clear to slightly opalescent and essentially free of foreign particles. The osmolality was about 310 mOsm/kg. The formulation is particularly suitable for injection, especially for subcutaneous injection. Moreover, a viscosity of about 9.6 mPas was measured, making it suitable for injection using a syringe. The conductivity at 20° C. was about 1.53 mS/cm, the density at 20° C. was about 1.067 g/cm³ and the density at 4° C. was about 1.071 g/cm³.

This 150 mg/ml risankizumab formulation may be provided as follows:

| | | | | In an embodiment |
|---|---|---|---|---|
| | | | | wherein this |
| | | | | formulation is |
| | | | | provided in a |
| | | | | syringe |
| | | | | (V = 1 ml) the |
| | Concen- | Concen- | | Nominal Amount |
| | tration | tration | | [mg/syringe] |
| Ingredient | [mmol/L] | [g/l] | Function | is as follows |
| Risankizumab | 1.00 | 150 | Drug | 150 |
| | | | substance | |
| Sodium acetate | 9.10 | 1.24 | Buffer | 1.24 |
| trihydrate | | | | |
| Acetic acid | 0.900 | 0.0540 | Buffer | 0.0540 |
| Trehalose | 185 | 70.0 | Adjustment | 70.0 |
| dihydrate | | | tonicity | |
| Polysorbate 20 | 0.163 | 0.200 | Surfactant | 0.200 |

| Abbreviation | Full form |
|---|---|
| APG | Acidic peak group |
| AUC | Area under curve |
| BPG | Basic peak group |
| CGE | Capillary gel electrophoresis |
| FNU | Formazine nephelometric units |
| F/T | Freeze/thaw |
| HIC | Hydrophobic interaction chromatography |
| HMW | High molecular weight |
| HP-SEC | High pressure size exclusion chromatography |
| IEC | Ion exchange chromatography |
| IL-23 | Interleukin-23 |
| LMW | Low molecular weight |
| MFI | Micro flow imaging |
| mOsm/kg | Milliosmole/kilogram |
| mPas | Millipascal second |
| mS/cm | Millisiemens per centimeter |
| PS20 | Polysorbate 20 |
| RALS | Right-angle light scattering |
| r.h. | Relative humidity |
| rhIL-23 | Recombinant human interleukin-23 |
| SEC | Size exclusion chromatography |
| SPR | Surface plasmon resonance |
| STP | Sampling time point |
| SVP | Sub-visible particles |
| UF/DF | Ultrafiltration/Diafiltration |
| U/min | Revolutions per minute |
| UP-SEC | Ultra-performance size exclusion chromatography |
| WCX | Weak cation exchange chromatography |

1-38. (canceled)

39. A stable liquid aqueous pharmaceutical formulation comprising
a) at least 150 mg/ml of risankizumab;
b) 150-200 mM trehalose;
c) 0.05-0.5 mg/ml a polysorbate; and
d) 5-15 mM acetate buffer.

40. The stable liquid aqueous pharmaceutical formulation of claim 39, comprising:
a) at least 150 mg/ml of risankizumab;
b) 170-200 mM trehalose;
c) 0.1-0.3 mg/ml the polysorbate; and
d) 7-12 mM acetate buffer.

41. The stable liquid aqueous pharmaceutical formulation of claim 39, wherein the stable liquid aqueous pharmaceutical formulation comprises 150 mg/ml to 192.3 mg/ml of risankizumab.

42. The stable liquid aqueous pharmaceutical formulation of claim 40, wherein the stable liquid aqueous pharmaceutical formulation comprises 150 mg/ml to 192.3 mg/ml of risankizumab.

43. The stable liquid aqueous pharmaceutical formulation of claim 39, wherein the polysorbate is polysorbate 20.

44. The stable liquid aqueous pharmaceutical formulation of claim 40, wherein the polysorbate is polysorbate 20.

45. The stable liquid aqueous pharmaceutical formulation of claim 39, wherein the stable liquid aqueous pharmaceutical formulation has a pH in the range of 5.5 to 6.2.

46. The stable liquid aqueous pharmaceutical formulation of claim 39, wherein the stable liquid aqueous pharmaceutical formulation has a pH in the range of 5.5 to 5.9.

47. The stable liquid aqueous pharmaceutical formulation of claim 39, wherein the viscosity of the formulation is less than 20.0 mPas measured at 200° C. following storage of the formulation for 12 months at 5° C.

48. The stable liquid aqueous pharmaceutical formulation of claim 39, wherein the stable liquid aqueous pharmaceutical formulation is formulated for subcutaneous administration.

49. A stable liquid aqueous pharmaceutical formulation, comprising:
a) risankizumab at a concentration from 150 mg/ml to 192.3 mg/ml;
b) 150-200 mM trehalose;
c) 0.05-0.5 mg/ml a polysorbate; and
d) 5-15 mM acetate buffer,
wherein the pH of the stable liquid aqueous pharmaceutical formulation is in the range of 5.5 to 6.2, and wherein the viscosity of the formulation is less than 20.0 mPas measured at 200° C.

50. The stable liquid aqueous pharmaceutical formulation of claim 49, comprising:
a) 170-200 mM trehalose;
b) 0.1-0.3 mg/ml the polysorbate; and
c) 7-12 mM acetate buffer

51. The stable liquid aqueous pharmaceutical formulation of claim 49, wherein the polysorbate is polysorbate 20.

52. The stable liquid aqueous pharmaceutical formulation of claim 50, wherein the polysorbate is polysorbate 20.

53. The stable liquid aqueous pharmaceutical formulation of claim 49, wherein the stable liquid aqueous pharmaceutical formulation has a pH in the range of 5.5 to 5.9.

54. The stable liquid aqueous pharmaceutical formulation of claim 50, wherein the stable liquid aqueous pharmaceutical formulation has a pH in the range of 5.5 to 5.9.

55. The stable liquid aqueous pharmaceutical formulation of claim 49, wherein the stable liquid aqueous pharmaceutical formulation is formulated for subcutaneous administration.

56. The stable liquid aqueous pharmaceutical formulation of claim 50, wherein the stable liquid aqueous pharmaceutical formulation is formulated for subcutaneous administration.

57. A stable liquid aqueous pharmaceutical formulation comprising
a) at least 150 mg/ml of risankizumab;
b) 150-200 mM trehalose;
c) 0.05-0.5 mg/ml a polysorbate; and
d) 5-15 mM acetate buffer;
wherein the stable liquid aqueous pharmaceutical formulation has a pH in the range of 5.5 to 6.2, and wherein the stable liquid aqueous pharmaceutical formulation is formulated for subcutaneous administration.

58. The stable liquid aqueous pharmaceutical formulation of claim 57, comprising:
a) 150 mg/ml to 192.3 mg/ml of risankizumab;
b) 170-200 mM trehalose;
c) 0.1-0.3 mg/ml polysorbate 20; and
d) 7-12 mM acetate buffer;
wherein the stable liquid aqueous pharmaceutical formulation has a pH in the range of 5.5 to 5.9.
